(19) United States
(12) Patent Application Publication
(10) Pub. No.: US 2023/0365955 A1
(43) Pub. Date: Nov. 16, 2023

(54) COMPOSITIONS AND METHODS FOR TREATMENT OF FABRY DISEASE

(71) Applicant: The Trustees of the University of Pennsylvania, Philadelphia, PA (US)

(72) Inventor: Juliette Hordeaux, Philadelphia, PA (US)

(21) Appl. No.: 18/246,872

(22) PCT Filed: Oct. 8, 2021
(86) PCT No.: PCT/US2021/054145

§ 371 Date: Mar. 28, 2023

Related U.S. Application Data
(60) Provisional application No. 63/186,092, filed on May 8, 2021.
(60) Provisional application No. 63/146,286, filed on Feb. 5, 2021.
(60) Provisional application No. 63/089,850, filed on Oct. 9, 2020.

(51) Int. Cl. C12N 9/40; C12N 15/86; A61P 43/00; A61K 48/00

(52) U.S. Cl. CPC C12N 9/2465; C12Y 302/01022; C12N 15/86; A61P 43/00; A61K 48/005; C12N 2750/14143; C12N 2830/50; C12N 2830/48

(57) ABSTRACT

Provided herein are polynucleotide sequences encoding functional human alpha-galactosidase A (hGLA) and expression cassettes containing these coding sequences. Also provided are vectors, such as recombinant adeno-associated virus (rAAV) vectors having vector genomes that include an hGLA coding sequence operably linked to one or more regulatory sequences. Further, compositions containing these expression cassettes and rAAV are provided, as well as methods for the use of these compositions for treatment of Fabry disease.

### BACKGROUND OF THE INVENTION

Fabry disease is an X-linked lysosomal disorder that results from mutations in the gene for the enzyme alpha-galactosidase A (GLA), which is responsible for the breakdown of globotriaosylceramide (GL-3 or Gb₃). Deficiencies in GLA result in the accumulation of GL-3 and related glycosphingolipids in the plasma and cellular lysosomes of vessels, nerves, tissues, and organs throughout the body. The disorder is a systemic disease, manifest as progressive renal failure, cardiac disease, cerebrovascular disease, small-fiber peripheral neuropathy, and skin lesions, among other abnormalities. GLA gene mutations that result in an absence of alpha-galactosidase A activity lead to the classic, severe form of Fabry disease. Mutations that decrease but do not eliminate the enzyme's activity usually cause the milder, late-onset forms of Fabry disease that typically affect only the heart or kidneys.

Standard treatments for Fabry disease currently include enzyme replacement therapy and medications to treat and prevent other symptoms of the disease. Kidney transplants may be needed in severe cases when renal failure occurs.

A need in the art exists for compositions and methods for safe and effective treatment of patients with Fabry disease.

### SUMMARY OF THE INVENTION

In one aspect, provide herein is a recombinant AAV (rAAV) comprising an AAVhu68 capsid having packaged therein a vector genome, wherein the vector genome comprises a coding sequence for a functional human alpha-galactosidase A (hGLA) and regulatory sequences which direct expression of the hGLA in a target cell, wherein the coding sequence comprises nucleotides 94 to 1287 of SEQ ID NO: 4, or a sequence at least 85% identical thereto, and wherein the hGLA has a cysteine residue at position 233 and/or position 359. In certain embodiments, the hGLA comprises at least amino acids 32 to 429 of SEQ ID NO: 2, or a sequence at least 95% identical thereto. In certain embodiments, the hGLA comprises amino acids 32 to 429 of SEQ ID NO: 7. In certain embodiments, wherein the hGLA comprises the native signal peptide. In other embodiments, hGLA comprises a heterologous signal peptide. In certain embodiments, the hGLA comprises the full length (amino acids 1 to 429) of SEQ ID NO: 17, or a sequence at least 95% identical thereto. In certain embodiments, the vector genome comprises a tissue-specific promoter. In certain embodiments, the regulatory sequences comprise a CB7 promoter, an intron, and a polyA. In certain embodiments, the regulatory sequences comprise a woodchuck hepatitis virus post-transcriptional regulatory element (WPRE). In certain embodiments, the vector genome comprises one or more miRNA target sequences.

In one aspect, provided herein is an expression cassette comprising a nucleic acid sequence encoding a functional human alpha-galactosidase A (hGLA) and one or more regulatory sequences which direct expression of the hGLA in a target cell containing the expression cassette, wherein the nucleic acid sequence comprises nucleotides 94 to 1287 of SEQ ID NO: 4, or a sequence at least 85% identical thereto, and wherein the hGLA has a cysteine residue at position 233 and/or position 359. In certain embodiments, the hGLA comprises amino acids 32 to 429 of SEQ ID NO: 7. In certain embodiments, the hGLA comprises the native signal peptide. In other embodiments, the hGLA comprises a heterologous signal peptide. In certain embodiments, the hGLA comprises the full-length (amino acids 1 to 429) of SEQ ID NO: 7, or a sequence at least 95% identical thereto. In certain embodiments, the expression cassette according to any one of claims 12 to 16, wherein the expression cassette comprises a tissue-specific promoter. In certain embodiments, the regulatory sequences comprise a CB7 promoter, an intron, and a polyA. In certain embodiments, the regulatory sequences comprise a woodchuck hepatitis virus post-transcriptional regulatory element (WPRE). In certain embodiments, the expression cassette comprises one or more miRNA target sequences.

In one aspect, provided herein is a plasmid comprising an expression cassette comprising a nucleic acid sequence encoding a functional human alpha-galactosidase A (hGLA) and one or more regulatory sequences which direct expression of the hGLA in a target cell containing the expression cassette, wherein the nucleic acid sequence comprises nucleotides 94 to 1287 of SEQ ID NO: 4, or a sequence at least 85% identical thereto, and wherein the hGLA has a cysteine residue at position 233 and/or position 359. In certain embodiments, the expression cassette is flanked by an AAV 5′ ITR and an AAV 3′ ITR. In further embodiments, a host cell containing an expression cassette or the plasmid is provided.

In yet another aspect, pharmaceutical compositions comprising a rAAV or an expression cassette comprising a nucleic acid sequence encoding a functional human alpha-galactosidase A (hGLA) is provided.

In another aspect, provided are methods of treating a human subject diagnosed with GLA-deficiency (Fabry disease) comprising administering to the subject a pharmaceutical composition comprising a rAAV or an expression cassette having a sequence that encodes a functional human alpha-galactosidase A (hGLA). In another aspect, rAAV, expression cassettes, and pharmaceutical composition for use in treatment of GLA-deficiency (Fabry disease) are provided.

Other aspects and advantages of the invention will be readily apparent from the following detailed description of the invention.

### BRIEF DESCRIPTION OF THE DRAWINGS

FIG. 1 shows a map for a CB7.CI.hGLAco(D233C_I359C).WPRE.rBG vector genome (SEQ ID NO: 6).

FIG. 2 shows a map for a CB7.CI.hGLAnat.WPRE.rBG vector genome (SEQ ID NO: 10).

FIG. 3 shows a map for a TBG.PI.hGLAnat.WPRE.bGH vector genome (SEQ ID NO: 8).

FIG. 4 shows a map for a CB7.CI.hGLAco.WPRE.rBG vector genome (SEQ ID NO: 14).

FIG. 5 shows a map for a TBG.PI.hGLAco.WPRE.bGH vector genome (SEQ ID NO: 12).

FIG. 6 shows a map for a CB7.CI.hGLAco(M51C_G360C).WPRE.rBG vector genome (SEQ ID NO: 18).

FIG. 7 shows a map for a TBG.PI.hGLAco(M51C_G360C).WPRE.bGH vector genome (SEQ ID NO: 16).

FIGS. 8A and 8B show an alignment of nucleotide sequences for hGLAnat (SEQ ID NO: 1), hGLAco (SEQ ID NO: 3), hGLAco(M51C_G360C) (SEQ ID NO: 5), hGLA(D233C_I359C) (SEQ ID NO: 4).

FIG. 9 shows an alignment of amino acid sequences for hGLAnat (SEQ ID NO: 2), hGLAco (SEQ ID NO: 13), hGLAco(M51C_G360C) (SEQ ID NO: 17), hGLA(D233C_I359C) (SEQ ID NO: 7).

FIG. 10A and FIG. 10B show body weights of untreated male and female control, Gla KO, WT/TgG3S, and Gla KO/TgG3S mice. Age-matched control (WT males and Gla HET females), Gla KO, WT/TgG3S, and Gla KO/TgG3S mice remained untreated to assess the natural history of these models. Body weights for male (FIG. 10A) and female (FIG. 10B) mice were recorded at 6 weeks, 12 weeks, 18 weeks, 25 weeks, 30 weeks, and 35 weeks of age. Average body weights are presented. Error bars represent the standard deviation. Abbreviations: Gla, alpha galactosidase A; TgG3S, human Gb3 synthase-transgenic.

FIG. 11A and FIG. 11B show hot plate response latencies of untreated male and female control, Gla KO, WT/TgG3S, and Gla KO/TgG3S mice. Age-matched control (WT males and Gla HET females), Gla KO, WT/TgG3S, and Gla KO/TgG3 S mice remained untreated to assess the natural history of these models. Sensitivity to heat stimuli in each mouse model was recorded as response latency (seconds) using the hotplate assay at 6 weeks, 12 weeks, 18 weeks, 25 weeks, 30 weeks, and 35 weeks. Data are expressed as the average recording among male (FIG. 11A) and female (FIG. 11B) mice at individual timepoints. Error bars represent the standard error of the mean.

FIG. 12A and FIG. 12B show blood urea nitrogen (BUN) concentrations of untreated male and female control, Gla KO, WT/TgG3S, and Gla KO/TgG3S mice. Age-matched control (WT males and Gla HET females), Gla KO, WT/TgG3S, and Gla KO/TgG3 S mice remained untreated to assess the natural history of these models. Blood urea nitrogen concentrations (mg/dL) were recorded at 6 weeks, 12 weeks, 18 weeks, 25 weeks, 30 weeks, and 35 weeks. Data are expressed as the average recording among male (FIG. 12A) and female (FIG. 12B) mice at individual timepoints. Error bars represent the standard error of the mean.

FIG. 13A and FIG. 13B show urine osmolality measured of male and female control, Gla KO, TgG3S, and Gla KO/TgG3S mice. Age-matched control (WT males and Gla HET females), Gla KO, WT/TgG3S, and Gla KO/TgG3 S mice remained untreated to assess the natural history of these models. Urine osmolality (mOsm/kg) was measured at 25 weeks, 30 weeks, and 35 weeks. Data are expressed as the average recording among male (FIG. 13A) and female (FIG. 13B) mice at individual timepoints. Error bars represent the standard error of the mean.

FIG. 14A and FIG. 14B show GL-3 storage in the kidney of male Gla KO, WT/TgG3S, and Gla KO/TgG3S. Age-matched control (WT males and Gla HET females), Gla KO, WT/TgG3S, Gla KO/TgG3S mice remained untreated to assess the natural history of these models. Kidneys and heart were harvested at necropsy and stained with an antibody recognizing GL-3 (dark precipitate) and a nuclear counterstain. (FIG. 14A) Representative IHC images from male mice are shown. Arrows in the kidney images indicate storage material in glomeruli. The region circle by a dotted line shows a focus of interstitial mononuclear inflammation (nephritis) seen only in some Gla KO/TgG3S mice. Arrows in heart images indicate cardiomyocyte necrosis and mineralization next adjacent to cardiomyocytes with GL-3 storage. FIG. 14B is a bar chart showing GL-3 storage throughout the kidney (percent area) was quantified using immunohistochemistry data. Results for male Gla KO, WT/TgG3S, and Gla KO/TgG3S mice are shown. **p<0.01 based on a Kruskal-Wallis test comparing groups to the WT/TgG3S controls.

FIG. 15A and FIG. 15B show GL-3 storage in the dorsal root ganglia (DRG) of male Gla KO, WT/TgG3S, and Gla KO/TgG3S. Age-matched control (WT males and Gla HET females), Gla KO, WT/TgG3S, Gla KO/TgG3 S mice remained untreated to assess the natural history of these models. DRG were harvested at necropsy and stained with an antibody recognizing GL-3 and a nuclear counterstain. (FIG. 15A) Representative images from male mice are shown. FIG. 15B is a bar chart showing GL-3 storage (percent area) in DRG was quantified using immunohistochemistry data. Results for male Gla KO, WT/TgG3S, and Gla KO/TgG3S mice are shown. **p<0.01 based on a Kruskal-Wallis test comparing groups to the WT/TgG3S controls.

FIG. 16A-FIG. 16D show quantification of lyso-Gb3 in plasma and GL-3 in tissues by LC-MS/MS in Gla KO, TgG3S, and Gla KO/TgG3S mice. Age-matched control (WT males and Gla HET females), Gla KO, WT/TgG3S, Gla KO/TgG3 S mice remained untreated to assess the natural history of these models. Kidney, heart, and brain tissue along with plasma were harvested at necropsy. LC-MS/MS was used to quantify GL3 in kidney (FIG. 16A), heart (FIG. 16B), and brain tissue (FIG. 16C) or lyso-Gb3 in plasma (FIG. 16D). For each of the figures, males and females are charted separately with the data from the female on bottom. *p<0.05, **p<0.01 Kruskal-Wallis test.

FIG. 17 shows transgene product expression (GLA enzyme activity) measured in blood serum at day 7 in Gla′ mice following administration of control (PBS) or one of three AAVhu68.hGLA vectors. Male and female mice 2 to 3 months of age were IV-administered PBS (control) or AAVhu68.hGLAnat, AAVhu68.hGLAco, or AAVhu68.hGLAco(M51C_G360C) at a dose of 1×10¹¹ GC (5.0×10¹² GC/kg) or 5×10¹¹ GC (2.5×10¹³ GC/kg). Blood was collected for serum isolation at 1 week and analyzed for GLA activity. The left graph show the aggregated data from all animals and the right and bottom graphs show results separated by gender.

FIG. 18 shows biodistribution of AAV genomic DNA measured in Gla^{-/-} mice following administration of control (PBS) or one of three AAVhu68.hGLA vectors. Male and female mice 2 to 3 months of age were IV-administered PBS (control) or AAVhu68.hGLAnat, AAVhu68.hGLAco, or AAVhu68.hGLAco(M51C_G360C) at a dose of 1×10¹¹ GC (5.0×10¹² GC/kg) or 5×10¹¹ GC (2.5×10¹³ GC/kg). Liver samples were collected at necropsy and analyzed for vector distribution. Results are expressed in GC of the transgene specific sequence relative to the amount of cellular genomic DNA.

FIG. 19 shows transgene product expression (GLA enzyme activity) levels in heart of Gla KO mice 28 days after administration of one of three AAVhu68.CB7.hGLA vectors. Male and female mice 2 to 3 months of age were IV-administered PBS (control) or AAVhu68.hGLAnat (hGLA), AAVhu68.hGLAco, or AAVhu68.hGLAco(M51C_G360C) at a dose of 1×10¹¹ GC (5.0×10¹² GC/kg) or 5×10¹¹ GC (2.5×10¹³ GC/kg). Heart samples were collected at necropsy and analyzed for GLA activity levels. The left graph shows the aggregated data from all animals and the middle and right plots show the results separated by gender.

FIG. 20 shows transgene product expression (GLA enzyme activity) in liver of Gla KO mice 28 days after administration of one of three AAVhu68.CB7.hGLA vectors. Male and female mice 2 to 3 months of age were IV-administered PBS (control) or AAVhu68.hGLAnat (hGLA), AAVhu68.hGLAco, or AAVhu68.hGLAco(M51C_G360C) at a dose of 1×10¹¹ GC (5.0×10¹² GC/kg) or 5×10¹¹ GC (2.5×10¹³ GC/kg). Liver samples were collected at necropsy and analyzed for GLA activity levels. The left graph shows the aggregated data from all animals and the middle and right plots show the results separated by gender.

FIG. 21 shows transgene product expression (GLA enzyme activity) in kidney of Gla KO mice 28 days after administration of one of three AAVhu68.CB7.hGLA vectors. Male and female mice 2 to 3 months of age were IV-administered PBS (control) or AAVhu68.hGLAnat (hGLA), AAVhu68.hGLAco, or AAVhu68.hGLAco(M51C_G360C) at a dose of 1×10¹¹ GC (5.0×10¹² GC/kg) or 5×10¹¹ GC (2.5×10¹³ GC/kg). Kidney samples were collected at necropsy and analyzed for GLA activity levels. The left graph shows the aggregated data from all animals and the middle and right plots show the results separated by gender.

FIG. 22 shows transgene product expression (GLA enzyme activity) in brain of Gla KO mice 28 days after administration of one of three AAVhu68.CB7.hGLA vectors. Male and female mice 2 to 3 months of age were IV-administered PBS (control) or AAVhu68.hGLAnat (hGLA), AAVhu68.hGLAco, or AAVhu68.hGLAco(M51C_G360C) at a dose of 1×10¹¹ GC (5.0×10¹² GC/kg) or 5×10¹¹ GC (2.5×10¹³ GC/kg). Brain samples were collected at necropsy and analyzed for GLA activity levels. The left graph shows the aggregated data from all animals and the middle and right plots show the results separated by gender.

FIG. 23 shows transgene product expression (GLA enzyme activity) in small intestine of Gla KO mice 28 days after administration of one of three AAVhu68.CB7.hGLA vectors. Male and female mice 2 to 3 months of age were IV-administered PBS (control) or AAVhu68.hGLAna (hGLA), AAVhu68.hGLAco, or AAVhu68.hGLAco(M51C_G360C) at a dose of 1×10¹¹ GC (5.0×10¹² GC/kg) or 5×10¹¹ GC (2.5×10¹³ GC/kg). Small intestine samples were collected at necropsy and analyzed for GLA activity levels. The top graph shows the aggregated data from all animals and the middle and bottom plots show the results separated by gender.

FIG. 24 shows lyso-Gb3 (globotriaosylsphingosine) storage in plasma and GL-3 storage in heart and kidney tissues of Gla KO mice following administration of one of three AAVhu68.CB7.hGLA vectors. Male and female mice 2 to 3 months of age were IV-administered PBS (control) or AAVhu68.hGLAnat (hGLA), AAVhu68.hGLAco, or AAVhu68.hGLAco(M51C_G360C) at a dose of 1×10¹¹ GC (5.0×10¹² GC/kg) or 5×10¹¹ GC (2.5×10¹³ GC/kg). Plasma was collected and the amount of storage material lyso-Gb3 was measured by LC-MS/MS (top graph). Kidney and heart samples were collected at necropsy and analyzed for GL-3 storage levels (middle and bottom graphs, respectively). The top graph shows the aggregated data from all animals and the middle and bottom plots show the results separated by gender.

FIG. 25 shows transgene product expression (GLA enzyme activity) in serum of Gla KO mice following administration of AAV vector or vehicle. Adult (3.5 to 4.5 months of age) male and female Gla KO or WT mice were IV-administered AAVhu68.hGLAco (WTco), AAVhu68.hGLAco(M51C_G360C) (AT #1), or AAVhu68.hGLAco(D233C_I359C) (AT #2) at a dose of 2.5×10¹² GC/kg (low-dose; LD), 5.0×10¹² GC/kg (mid-dose; MD), or 2.5×10¹³ GC/kg (high-dose; HD, only for AAVhu68.hGLAco(D233C_I359C)). Additional Gla KO or WT mice were IV-administered vehicle (PBS) as a control. Serum samples were collected 7 days post administration and analyzed for transgene product expression (GLA enzyme activity). Aggregated data from all animals are presented, along with data separated by sex. Results for vehicle-treated WT and Gla KO mice are historical data and are included for reference. The historical GLA enzyme activity values from both WT and Gla KO mouse samples were all below quantifiable limits, so no data points are graphed. HD, high-dose; LD, low-dose; MD, mid-dose.

FIG. 26 shows transgene product expression (GLA enzyme activity) in plasma of Gla KO mice following administration of AAV vector or vehicle. Adult (3.5 to 4.5 months of age) male and female Gla KO or WT mice were IV-administered AAVhu68.hGLAco (WTco), AAVhu68.hGLAco(M51C_G360C) (AT #1), or AAVhu68.hGLAco(D233C_I359C) (AT #2) at a dose of 2.5×10¹² GC/kg (low-dose; LD), 5.0×10¹² GC/kg (mid-dose; MD), or 2.5×10¹³ GC/kg (high-dose; HD, only for AAVhu68.hGLAco(D233C_I359C)). Additional Gla KO or WT mice were IV-administered vehicle (PBS) as a control. Plasma samples were collected 28 days post injection and analyzed for transgene product expression (GLA enzyme activity). The top graphs show the aggregated data from all animals and the middle and bottom plots show the results separated by gender.

FIG. 27 shows transgene product expression (GLA enzyme activity) in heart of Gla KO mice following administration of AAV vector or vehicle. Adult (3.5 to 4.5 months of age) male and female Gla KO or WT mice were IV-administered AAVhu68.hGLAco (WTco), AAVhu68.hGLAco(M51C_G360C) (AT #1), or AAVhu68.hGLAco(D233C_I359C) (AT #2) at a dose of 2.5×10¹² GC/kg (low-dose; LD), 5.0×10¹² GC/kg (mid-dose; MD), or 2.5×10¹³ GC/kg (high-dose; HD, only for AAVhu68.hGLAco(D233C_I359C)). Additional Gla KO or WT mice were IV-administered vehicle (PBS) as a control. Heart samples were collected at necropsy and analyzed for transgene product expression (GLA enzyme activity). The top graphs show the aggregated data from all animals and the middle and bottom plots show the results separated by gender. Results for vehicle-treated WT and Gla KO mice are historical data and are included for reference. The historical GLA enzyme activity values from both WT and Gla KO mouse samples were all below quantifiable limits, so no data points are graphed.

FIG. 28 shows transgene product expression (GLA enzyme activity) in liver of Gla KO mice following administration of AAV vector or vehicle. Adult (3.5 to 4.5 months of age) male and female Gla KO or WT mice were IV-administered AAVhu68.hGLAco (WTco), AAVhu68.hGLAco(M51C_G360C) (AT #1), or AAVhu68.hGLAco(D233C_I359C) (AT #2) at a dose of 2.5×10¹² GC/kg (low-dose; LD), 5.0×10¹² GC/kg (mid-dose; MD), or 2.5×10¹³ GC/kg (high-dose; HD, only for AAVhu68.hGLAco(D233C_I359C)). Additional Gla KO or WT mice were IV-administered vehicle (PBS) as a control. Liver samples were collected at necropsy and analyzed for transgene product expression (GLA enzyme activity). The top graphs show the aggregated data from all animals and the middle and bottom plots show the results separated by gender. Results for vehicle-treated WT and Gla KO mice are historical data and are included for reference. The historical GLA enzyme activity values from both WT and Gla KO mouse samples were all below quantifiable limits, so no data points are graphed.

FIG. 29 shows transgene product expression (GLA enzyme activity) in kidney of Gla KO mice following administration of AAV vector or vehicle. Adult (3.5 to 4.5 months of age) male and female Gla KO or WT mice were IV-administered AAVhu68.hGLAco (WTco), AAVhu68.hGLAco(M51C_G360C) (AT #1), or AAVhu68.hGLAco(D233C_I359C) (AT #2) at a dose of 2.5×10¹² GC/kg (low-dose; LD), 5.0×10¹² GC/kg (mid-dose; MD), or 2.5×10¹³ GC/kg (high-dose; HD, only for AAVhu68.hGLAco(D233C-I359C)). Additional Gla KO or WT mice were IV-administered vehicle (PBS) as a control. Kidney samples were collected at necropsy and analyzed for transgene product expression (GLA enzyme activity). The top graphs show the aggregated data from all animals and the middle and bottom plots show the results separated by gender. Results for vehicle-treated WT and Gla KO mice are historical data and are included for reference. The historical GLA enzyme activity values from both WT and Gla KO mouse samples were all below quantifiable limits, so no data points are graphed.

FIG. 30 shows lyso-Gb3 (globotriaosylsphingosine) storage in plasma collected from Gla KO mice following administration of administration of AAV vector or vehicle. Adult (3.5 to 4.5 months of age) male and female Gla KO or WT mice were IV-administered AAVhu68.hGLAco (WTco), AAVhu68.hGLAco(M51C_G360C) (AT #1), or AAVhu68.hGLAco(D233C_I359C) (AT #2) at a dose of 2.5×10¹² GC/kg (low-dose; LD), 5.0×10¹² GC/kg (mid-dose; MD), or 2.5×10¹³ GC/kg (high-dose; HD, only for AAVhu68.hGLAco(D233C_I359C)). Additional Gla KO or WT mice were IV-administered vehicle (PBS) as a control. Plasma samples were collected 28 days post administration at necropsy and analyzed for lyso-Gb3 storage levels. The top graphs show the aggregated data from all animals and the middle and bottom plots show the results separated by gender. Results for vehicle-treated WT and Gla KO mice are historical data and are included for reference.

FIG. 31A and FIG. 31B show GL-3 (globotriaosylceramide) storage in the kidney of Gla KO mice following administration of administration of AAV vector or vehicle. Adult (3.5 to 4.5 months of age) male and female Gla KO or WT mice were IV-administered AAVhu68.hGLAco, AAVhu68.hGLAco(M51C_G360C) (eng #1), or AAVhu68.hGLAco(D233C_I359C) (eng #2) at a dose of 2.5×10¹² GC/kg (low-dose; LD), 5.0×10¹² GC/kg (mid-dose; MD), or 2.5×10¹³ GC/kg (high-dose; HD, only for AAVhu68.hGLAco(D233C_I359C)). Additional Gla KO or WT mice were IV-administered vehicle (PBS) as a control. (FIG. 31A) Kidneys were harvested at necropsy and stained with an antibody recognizing GL-3 (globotriaosylceramide, arrows). Representative images from males are shown and labeled. FIG. 31B is a bar chart providing quantification of GL-3+IHC signal showing the percentage of tubules with GL-3+ deposits. *p<0.05, **p<0.01, *** p<0.001, ****p<0.0001 based on a Kruskal-Wallis test followed by post-hoc Dunn's multiple comparisons test comparing groups to the vehicle-treated Gla KO mice.

FIG. 32A and FIG. 32B show GL-3 (globotriaosylceramide) storage in the DRG of Gla KO following administration of administration of AAV vector or vehicle. Adult (3.5 to 4.5 months of age) male and female Gla KO or WT mice were IV-administered AAVhu68.hGLAco, AAVhu68.hGLAco(M51C_G360C) (eng #1), or AAVhu68.hGLAco(D233C_I359C) (eng #2) at a dose of 2.5×10¹² GC/kg (low-dose; LD), 5.0×10¹² GC/kg (mid-dose; MD), or 2.5×10¹³ GC/kg (high-dose; HD, only for AAVhu68.hGLAco(D233C_I359Cco)). Additional Gla KO or WT mice were IV-administered vehicle (PBS) as a control. (FIG. 32A) DRG were harvested at necropsy with spinal cords and stained with an antibody recognizing GL-3 (globotriaosylceramide, dark precipitate). Representative images from males are shown and labeled. FIG. 32B is a bar chart showing quantification of GL-3+ IHC signal by percent GL-3+ area is shown. *p<0.05, **p<0.01, *** p<0.001, ****p<0.0001 based on a Kruskal-Wallis test followed by post-hoc Dunn's multiple comparisons test comparing groups to the vehicle-treated Gla KO mice.

FIG. 33 shows a western blot analysis of in vivo secreted GLA in plasma from AAV treated animals that were administered AAVhu68.hGLAco (hGLAco), AAVhu68.hGLAco(M51C_G360C) (hGLA eng #1), or AAVhu68.hGLAco(D233C_I359C) (hGLA eng #2).

FIG. 34A and FIG. 34B show cardiac transduction and expression of hGLA stained by immunohistochemistry in Gla KO male mice (FIG. 34A) and female mice (FIG. 34B) treated with AAVhu68.hGLAco(D233C_I359C). 3.5 to 4.5 months old GLA KO Fabry mice were injected IV with low-dose—LD (2.5×10¹² GC/kg), mid-dose—MD (5×10¹² 12 GC/kg) or high-dose—HD (2.5×10¹³ GC/kg) of either AAVhu68.hGLAco, AAVhu68.hGLAco(M51C_G360C), or AAVhu68.hGLAco(D233C_I359C). Mice were euthanized 4 weeks post injection and tissues were collected. Hearts were zinc-formalin-fixed and paraffin embedded. An antibody to hGLA was used to stain transgene expression. Representative pictures from animals injected with AAVhu68.CB7.hGLAco(D233C_I359C) are shown. Dark immunostaining of hGLA shows robust and dose-dependent transgene expression in cardiomyocytes from ventricles and atria.

FIG. 35 shows anti-GLA titers in plasma of Gla KO mice following IV administration of a LD (2.5×10¹² GC/kg), MD (5×10¹² GC/kg), or HD (2.5×10¹³ GC/kg) of AAVhu68. hGLAco (hGLAco), AAVhu68.hGLAco(M51C_G360C) (hGLA eng #1), or AAVhu68.hGLAco(D233C_I359C) (hGLA eng #2).

FIG. 36A and FIG. 36B show AST and ALT concentrations in adult NHPs following a single IV dose of AAVhu68.hGLAco(D233C_I359C) (hGLA eng #2). Adult NHPs (N=4) received a single IV administration of AAVhu68.hGLAco(D233C_I359C) (hGLA eng #2) at a dose of 2.5×10¹³ GC/kg. Blood was collected at baseline, Day 0, Day 3, Day 7, Day 14, Day 28, and Day 60 and analyzed for AST (FIG. 36A) and ALT (FIG. 36B) concentration. The dotted line represents the reference values. Abbreviations: ALT, alanine aminotransferase; AST, aspartate aminotransferase; GC, genome copies; GGT, gamma-glutamyl transferase.

FIG. 37A-FIG. 37C show total bilirubin (TBil) levels, platelet count, and white blood cell (WBC) count in adult NHPs following a single IV dose of AAVhu68.hGLAco(D233C_I359C) (hGLA eng #2). Adult NHPs (N=4) received a single IV administration of AAVhu68.hGLAco(D233C_I359C) (hGLA eng #2) at a dose of 2.5×10¹³ GC/kg. Blood was collected at baseline, Day 0, Day 3, Day 7, Day 14, Day 28, and Day 60 and analyzed for TBil levels (FIG. 37A), platelet count (FIG. 37B), and WBC count (FIG. 37C). The dotted lines represent the reference values.

FIG. 38A-FIG. 38C show PT (prothrombin time), APTT (activated partial thromboplastin time), and D-Dimer levels in adult NHPs following a single IV dose of AAVhu68.hGLAco(D233C_I359C) (hGLA eng #2). Adult NHPs (N=4) received a single IV administration of AAVhu68.hGLAco(D233C_I359C) (hGLA eng #2) at a dose of 2.5×10¹³ GC/kg. Blood was collected at baseline, Day 0, Day 3, Day 7, Day 14, Day 28, and Day 60 and analyzed for PT (FIG. 38A), APTT (FIG. 38B), and D-dimer levels (FIG. 38C).

FIG. 39 show neutralizing antibodies and non-neutralizing binding antibodies in Adult NHPs following a single IV dose of AAVhu68.hGLAco(D233C_I359C) (hGLA eng #2). Abbreviations: Babs=non-neutralizing binding antibodies; F=female; ID=identification; M=male; Nab=neutralizing antibodies; NHP=non-human primate. a—Values are the serum reciprocal dilution at which relative luminescence units (RLUs) was reduced 50% compared to virus control wells (no test sample). b—Values are the reciprocal of the highest serum dilution that produced a mean OD450 value 3× greater than a negative control serum. c—IgG and IgM are BAbs.

FIG. 40 shows transgene product expression (GLA enzyme activity) in plasma of adult NHPs following a single intravenous administration of AAVhu68.hGLAco(D233C_I359C) (hGLA eng #2). Adult NHPs (n=4) received a single IV dose of AAVhu68.hGLAco(D233C_I359C) (hGLA eng #2) at a dose of 2.5×10¹³ GC/kg. Plasma was collected on Day 7, Day 14, Day 28, and Day 60. Transgene product expression (GLA enzyme activity) was measured. The dashed line represents the baseline titer.

FIG. 41 shows antibodies against the transgene product (anti-GLA antibodies) in plasma of adult NHPs following a single intravenous administration of AAVhu68.hGLAco(D233C_I359C) (hGLA eng #2). Adult NHPs (n=4) received a single IV dose of AAVhu68.hGLAco(D233C_I359C) (hGLA eng #2) at a dose of 2.5×10¹³ GC/kg. Plasma was collected on Day 7, Day 14, Day 28, and Day 60. Antibodies against the transgene product (anti-GLA antibodies) were measured. The dashed line represents the baseline enzyme activity.

FIG. 42A and FIG. 42B show transgene product expression (GLA enzyme activity) in heart, liver, and kidney of adult NHPs following a single intravenous administration of AAVhu68.hGLAco(D233C_I359C) (hGLA eng #2). Adult NHPs (n=4) received a single IV dose of AAVhu68.hGLAco(D233C_I359C) (hGLA eng #2) at a dose of 2.5×10¹³ GC/kg. On Day 60, animals were necropsied and the heart, liver, and kidney were collected to measure transgene product expression (GLA enzyme activity) (FIG. 42A). Heart tissue from an untreated wild type NHP of the same species (cynomolgus macaque) was supplied by BioIVT as a comparator for baseline GLA enzyme activity (dashed line). Fold increases in GLA enzyme activity were calculated based on measurements (FIG. 42B).

FIG. 43 shows representative images of ISH for transgene and IHC for GLA expression in kidney, DRG, and heart tissue from NHP following administration of AAVhu68.hGLAco(D233C-I359C) (hGLA eng #2).

FIG. 44 shows representative images of ISH for transgene expression (RNAscope probes) and GLA expression in heart tissue from NHP following administration of AAVhu68.hGLAco(D233C-I359C) (hGLA eng #2).

FIG. 45 shows representative images of ISH for transgene expression (RNAscope probes) and GLA expression in DRG from NHP following administration of AAVhu68.hGLAco(D233C-I359C) (hGLA eng #2).

### DETAILED DESCRIPTION OF THE INVENTION

Provided herein are compositions useful for the treatment of Fabry disease and/or alleviating symptoms of Fabry disease are provided herein.

Without wishing to be bound by theory, including regular infusion of recombinant human α-Gal A (rhα-Gal A), termed enzyme replacement therapy (ERT), is currently the primary treatment option for Fabry patients with non-amenable mutations, whereas patients with amenable mutations can benefit from both ERT and small molecule chaperones. However, rha-Gal A has low physical stability, a short circulating half-life, and variable uptake into different disease-relevant tissues, which may limit the efficacy of ERT as well as gene therapies relying on cross correction. The compositions provided herein deliver stabilized hGLA that are effective for gene therapy and provide a larger window for the enzyme to stay active while in circulation prior to being taken up into the target tissues.

In certain embodiments, the compositions and methods described herein include nucleic acid sequences, expression cassettes, vectors, recombinant viruses, and other compositions and methods for expression of a functional hGLA. In certain embodiments, the compositions and methods described herein include nucleic acid sequences, expression cassettes, vectors, recombinant viruses, host cells, other compositions and methods for production of a composition comprising either a nucleic acid sequence encoding a functional hGLA or a hGLA polypeptide. In yet another embodiment, the compositions and methods described herein include nucleic acid sequences, expression cassettes, vectors, recombinant viruses, other compositions and methods for delivery of the nucleic acid sequence encoding a functional hGLA to a subject for the treatment of Fabry disease. In one embodiment, the compositions and methods described herein are useful for providing therapeutic levels of hGLA in the periphery, such as, e.g., blood, liver, kidney, and/or peripheral nervous system of a subject. In certain embodiments, an adeno-associated viral (AAV) vector-based method described herein provides a new treatment option, helping to restore a desired function of hGLA and to alleviate a symptoms associated with hGLA-deficiency (Fabry disease) by providing expression of a hGLA in a subject in need thereof.

As used herein, the term “a therapeutic level” means an enzyme activity at least about 5%, about 10%, about 20%, about 25%, about 30%, about 35%, about 40%, about 45%, about 50%, about 55%, about 60%, about 65%, about 70%, about 75%, about 80%, about 85%, about 90%, about 95%, about 100%, more than 100%, about 2-fold, about 3-fold, or about 5-fold of a healthy control. Suitable assays for measuring hGLA enzymatic activity are known to those of skill in the art. In some embodiments, such therapeutic levels of hGLA may result in alleviation of Fabry disease-related symptoms; improvement of Fabry disease-related biomarkers of disease (for example, reduction of Gb3 levels in serum, urine and/or other biological samples); facilitation of other treatment(s) for Fabry disease (e.g., enzyme replacement or chaperone therapy); prevention of neurocognitive decline; reversal of certain Fabry disease-related symptoms and/or prevention of progression of Fabry disease-related symptoms; or any combination thereof.

As used herein, “a healthy control” refers to a subject or a biological sample therefrom, wherein the subject does not have Fabry disease or hGLA deficiency otherwise. The healthy control can be from one subject. In another embodiment, the healthy control is a pooled sample from multiple subjects.

As used herein, the term “biological sample” refers to any cell, biological fluid, or tissue. Suitable samples for use in this invention may include, without limitation, whole blood, leukocytes, fibroblasts, serum, urine, plasma, saliva, bone marrow, cerebrospinal fluid, amniotic fluid, and skin cells. Such samples may further be diluted with saline, buffer, or a physiologically acceptable diluent. Alternatively, such samples are concentrated by conventional means.

With regard to the description herein, it is intended that each of the vectors and other compositions herein described, is useful, in another embodiment. In addition, it is also intended that each of the compositions herein described as useful in the methods, is, in another embodiment, itself an embodiment of the invention.

Unless defined otherwise in this specification, technical and scientific terms used herein have the same meaning as commonly understood by one of ordinary skill in the art to which this invention belongs and by reference to published texts, which provide one skilled in the art with a general guide to many of the terms used in the present application.

As used herein, “disease,” “disorder,” and “condition” refer to Fabry disease and/or hGLA deficiency in a subject.

As used herein, the term “Fabry-related symptom(s)” or “symptom(s)” refers to symptom(s) found in patients with Fabry disease as well as in animal models for Fabry disease. Such symptoms include but are not limited to angiokeratomas, acroparesthesia, hypohidrosis/anhidrosis, corneal, lenticular opacity, cardiac problems, pain, and a reduction in kidney function. Further, common cardiac-related signs and symptoms of Fabry disease include left ventricular hypertrophy, valvular disease (especially mitral valve prolapse and/or regurgitation), premature coronary artery disease, angina, myocardial infarction, conduction abnormalities, arrhythmias, congestive heart failure. Fabry disease is referred to by other names including alpha-galactosidase A deficiency, Anderson-Fabry disease, and angiokeratoma corporis diffusum.

“Patient” or “subject” as used herein refers to a male or female human, dogs, and animal models used for clinical research. In certain embodiments, the subject of these methods and compositions is a human diagnosed with Fabry disease. In further embodiments, the human subject of these methods and compositions is a prenatal, a newborn, an infant, a toddler, a preschool-aged child, a grade-school-aged child, a teen, a young adult, or an adult.

“Comprising” is a term meaning inclusive of other components or method steps. When “comprising” is used, it is to be understood that related embodiments include descriptions using the “consisting of” terminology, which excludes other components or method steps, and “consisting essentially of” terminology, which excludes any components or method steps that substantially change the nature of the embodiment or invention. It should be understood that while various embodiments in the specification are presented using “comprising” language, under various circumstances, a related embodiment is also described using “consisting of” or “consisting essentially of” language.

A reference to “one embodiment”, “another embodiment”, or “a certain embodiment” in describing an embodiment does not imply that the referenced embodiment is mutually exclusive with another embodiment (e.g., an embodiment described before the referenced embodiment), unless expressly specified otherwise.

It is to be noted that the term “a” or “an”, refers to one or more, for example, “an expression cassette”, is understood to represent one or more expression cassette (s). As such, the terms “a” (or “an”), “one or more,” and “at least one” are used interchangeably herein.

As used herein, the term “about” means a variability of plus or minus 10% from the reference given, unless otherwise specified.

1. Human Alpha Galactosidase A (hGLA)

As used herein, the terms “human alpha galactosidase A” and “hGLA” are used interchangeably to refer to a human alpha galactosidase A enzyme. Alternative names for alpha galactosidase A include agalsidase alfa, alpha-D-galactosidase A, alpha-D-galactoside galactohydrolase, alpha-galactosidase, alpha-galactosidase A, ceramidetrihexosidase, GALA, galactosidase, alpha, and melibiase. It will be understood that the Greek letter “alpha” and the symbol “a” are used interchangeably throughout this specification. Included are native (wild-type) hGLA proteins and, in particular, variant hGLA proteins expressed from the nucleic acid sequences provided herein, or functional fragments thereof, which restore a desired function, ameliorate symptoms, improve symptoms associated with a Fabry disease-related biomarker (e.g. serum alpha-GAL), and/or facilitate other treatment(s) for Fabry disease when delivered in a composition or by a method as provided herein.

The “human alpha galactosidase A” or “hGLA” may be, for example, a full-length protein (including a signal peptide and the mature protein), the mature protein, a variant protein as described herein, or a functional fragment. As used herein, the term “functional hGLA” refers to an enzyme having the amino acid sequence of the full-length native (wild-type) protein (as shown in SEQ ID NO: 2 and UniProtKB accession number: P06280-1), a variant thereof (including those described herein with specific amino acid substitution(s)), a mutant thereof with a conservative amino acid replacement, a fragment thereof, a full-length or a fragment of any combination of the variant and the mutant with a conservative amino acid replacement, which provides at least about 10%, at least about 20%, at least about 30%, at least about 40%, at least about 50%, at least about 60%, at least about 70%, at least about 75%, at least about 80%, at least about 90%, or about the same, or greater than 100% of the biological activity level of a native (wild-type) hGLA.

```
human Alpha-galactosidase A - signal peptide (amino acids 1 to 31)
(SEQ ID NO: 2)
                10 20 30 40 50
MQLRNPELHL GCALALRFLA LVSWDIPGAR ALDNGLARTP TMGWLHWERF
                60 70 80 90 100
MCNLDCQEEP DSCISEKLFM EMAELMVSEG WKDAGYEYLC IDDCWMAPQR
              110 120 130 140 150
DSEGRLQADP QRFPHGIRQL ANYVHSKGLK LGIYADVGNK TCAGFPGSFG
              160 170 180 190 200
YYDIDAQTFA DWGVDLLKFD GCYCDSLENL ADGYKHMSLA LNRTGRSIVY
              210 220 230 240 250
SCEWPLYMWP FQKPNYTEIR QYCNHWRNFA DIDDSWKSIK SILDWTSFNQ
              260 270 280 290 300
ERIVDVAGPG GWNDPDMLVI GNFGLSWNQQ VTQMALWAIM AAPLFMSNDL
              310 320 330 340 350
RHISPQAKAL LQDKDVIAIN QDPLGKQGYQ LRQGDNFEVW ERPLSGLAWA
              360 370 380 390 400
VAMINRQEIG GPRSYTIAVA SLGKGVACNP ACFITQLLPV KRKLGFYEWT
              410 420
SRLRSHINPT GTVLLQLENT MQMSLKDLL
Native human GLA coding sequence (see NCBI Reference Sequence:
NM_000169.) signal peptide (nucleotides 1 to 93)
(SEQ ID NO: 1)
atgcagct gaggaaccca gaactacatc tgggctgcgc
gcttgcgctt cgcttcctgg ccctcgtttc ctgggacatc cctggggcta gagcactgga
caatggattg gcaaggacgc ctaccatggg ctggctgcac tgggagcgct tcatgtgcaa
ccttgactgc caggaagagc cagattcctg catcagtgag aagctcttca tggagatggc
agagctcatg gtctcagaag gctggaagga tgcaggttat gagtacctct gcattgatga
ctgttggatg gctccccaaa gagattcaga aggcagactt caggcagacc ctcagcgctt
tcctcatggg attcgccagc tagctaatta tgttcacagc aaaggactga agctagggat
ttatgcagat gttggaaata aaacctgcgc aggcttccct gggagttttg gatactacga
cattgatgcc cagacctttg ctgactgggg agtagatctg ctaaaatttg atggttgtta
ctgtgacagt ttggaaaatt tggcagatgg ttataagcac atgtccttgg ccctgaatag
gactggcaga agcattgtgt actcctgtga gtggcctctt tatatgtggc cctttcaaaa
gcccaattat acagaaatcc gacagtactg caatcactgg cgaaattttg ctgacattga
tgattcctgg aaaagtataa agagtatctt ggactggaca tcttttaacc aggagagaat
tgttgatgtt gctggaccag ggggttggaa tgacccagat atgttagtga ttggcaactt
tggcctcagc tggaatcagc aagtaactca gatggccctc tgggctatca tggctgctcc
tttattcatg tctaatgacc tccgacacat cagccctcaa gccaaagctc tccttcagga
taaggacgta attgccatca atcaggaccc cttgggcaag caagggtacc agcttagaca
gggagacaac tttgaagtgt gggaacgacc tctctcaggc ttagcctggg ctgtagctat
gataaaccgg caggagattg gtggacctcg ctcttatacc atcgcagttg cttccctggg
taaaggagtg gcctgtaatc ctgcctgctt catcacacag ctcctccctg tgaaaaggaa
gctagggttc tatgaatgga cttcaaggtt aagaagtcac ataaatccca caggcactgt
tttgcttcag ctagaaaata caatgcagat gtcattaaaa gacttactt
```

With reference to the numbering of the full-length native hGLA of SEQ ID NO: 2, there is a signal peptide at amino acid positions 1 to 31 and the mature protein includes amino acid 32 to 429. As used herein, a “signal peptide” refers to a short peptide (usually about 16 to 35 amino acids) present at the N-terminus of newly synthesized proteins. A signal peptide, and in some cases the nucleic acid sequences encoding such a peptide, may also be referred to as a signal sequence, a targeting signal, a localization signal, a localization sequence, a transit peptide, a leader sequence, or a leader peptide. As described herein, an hGLA may include a native signal peptide (i.e. amino acids 1 to 31 of SEQ ID NO: 2) or, alternatively, a heterologous signal peptide. In certain embodiments, the hGLA is a mature protein (lacking a signal peptide sequence).

In certain embodiments, a hGLA includes a heterologous signal peptide. In certain embodiments, such a heterologous signal peptide is preferably of human origin and may include, e.g., an IL-2 signal peptide. Particular heterologous signal peptides workable in the certain embodiments include amino acids 1-20 from chymotrypsinogen B2, the signal peptide of human alpha-1-antitrypsin, amino acids 1-25 from iduronate-2-sulphatase, and amino acids 1-23 from protease CI inhibitor. See, e.g., WO2018046774. Other signal/leader peptides may be natively found in an immunoglobulin (e.g., IgG), a cytokine (e.g., IL-2, IL12, IL18, or the like), insulin, albumin, β-glucuronidase, alkaline protease or the fibronectin secretory signal peptides, amongst others. See, also, e.g., signalpeptide.de/index.php?m=listspdb_mammalia. Such a chimeric hGLA may have the heterologous leader in the place of the entire 31 amino acid native signal peptide. Optionally, an N-terminal truncation of the hGLA enzyme may lack only a portion of the signal peptide (e.g., a deletion of about 2 to about 25 amino acids, or values therebetween), the entire signal peptide, or a fragment longer than the signal peptide (e.g., up to amino acids 70 based on the numbering of SEQ ID NO: 2. Optionally, such an enzyme may contain a C-terminal truncation of about 5, 10, 15, or 20 amino acids in length.

In certain embodiments, an hGLA may be selected which has a sequence that is at least 95% identical, at least 97% identical, or at least 99% identical to the full-length (amino acids 1 to 429) of SEQ ID NO: 2. In certain embodiments, provided is a sequence which is at least 95%, at least 97%, or at least 99% identical to the mature protein (amino acids 32 to 429) of SEQ ID NO: 2. In certain embodiments, the sequence having at least 95% to at least 99% identity to the hGLA of either the full-length (amino acids 1 to 429) or mature protein (amino acids 32 to 429) is characterized by having an improved biological effect and better safety profile than the reference (i.e. native) hGLA when tested in an appropriate animal model. In certain embodiments, the hGLA enzyme contains modifications in designated positions in the hGLA amino acid sequence. For example, in certain embodiments, the hGLA has a cysteine substitution at position 51 and/or position 360, with respect to the numbering in SEQ ID NO: 2. In certain embodiments the hGLA has a cysteine substitution at positions 233 and/or position 359, with respect to the numbering in SEQ ID NO: 2. Examples of such hGLA polypeptides are provided in SEQ ID NO: 7 and 17.

As used herein, the “conservative amino acid replacement” or “conservative amino acid substitutions” refers to a change, replacement or substitution of an amino acid to a different amino acid with similar biochemical properties (e.g. charge, hydrophobicity and size), which is known by practitioners of the art. Also see, e.g. FRENCH et al. What is a conservative substitution? Journal of Molecular Evolution, March 1983, Volume 19, Issue 2, pp 171-175 and YAMPOLSKY et al. The Exchangeability of Amino Acids in Proteins, Genetics. 2005 August; 170(4): 1459-1472, each of which is incorporated herein by reference in its entirety.

In one aspect, provide herein are nucleic acid sequences and, for example, expression cassettes and vectors comprising the same, which encode a functional hGLA protein. In one embodiment, the nucleic acid sequence is the wild-type coding sequence reproduced in SEQ ID NO: 1. In further embodiments, the nucleic acid sequence is at least about 60%, at least about 65%, at least about 70%, at least about 75%, or at least about 80% identical to the wild type hGLA sequence of SEQ ID NO: 1, and encodes a functional hGLA.

As used herein, “a nucleic acid” refers to a polymeric form of nucleotides and includes RNA, mRNA, cDNA, genomic DNA, peptide nucleic acid (PNA) and synthetic forms and mixed polymers of the above. A nucleotide refers to a ribonucleotide, deoxynucleotide or a modified form of either type of nucleotide (e.g., a peptide nucleic acid oligomer). The term also includes single- and double-stranded forms of DNA. The skilled person will appreciate that functional variants of these nucleic acid molecules are described herein. Functional variants are nucleic acid sequences that can be directly translated, using the standard genetic code, to provide an amino acid sequence identical to that translated from a parental nucleic acid molecule.

In certain embodiments, the nucleic acid molecules encoding a functional hGLA, and other constructs as described herein are useful in generating expression cassettes and vector genomes and may be engineered for expression in yeast cells, insect cells, or mammalian cells, such as human cells. Methods are known and have been described previously (e.g. WO 96/09378). A sequence is considered engineered if at least one non-preferred codon as compared to a wild type sequence is replaced by a codon that is more preferred. Herein, a non-preferred codon is a codon that is used less frequently in an organism than another codon coding for the same amino acid, and a codon that is more preferred is a codon that is used more frequently in an organism than a non-preferred codon. The frequency of codon usage for a specific organism can be found in codon frequency tables, such as in www.kazusa.jp/codon. Preferably more than one non-preferred codon, preferably most or all non-preferred codons, are replaced by codons that are more preferred. Preferably the most frequently used codons in an organism are used in an engineered sequence. Replacement by preferred codons generally leads to higher expression. It will also be understood by a skilled person that numerous different nucleic acid molecules can encode the same polypeptide as a result of the degeneracy of the genetic code. It is also understood that skilled persons may, using routine techniques, make nucleotide substitutions that do not affect the amino acid sequence encoded by the nucleic acid molecules to reflect the codon usage of any particular host organism in which the polypeptides are to be expressed. Therefore, unless otherwise specified, a “nucleic acid sequence encoding an amino acid sequence” includes all nucleotide sequences that are degenerate versions of each other and that encode the same amino acid sequence. Nucleic acid sequences can be cloned using routine molecular biology techniques, or generated de novo by DNA synthesis, which can be performed using routine procedures by service companies having business in the field of DNA synthesis and/or molecular cloning (e.g. GeneArt, GenScript, Life Technologies, Eurofins).

In certain embodiments, the nucleic acids, expression cassettes, vector genomes described herein include an hGLA coding sequence that is an engineered sequence. In certain embodiments, the engineered sequence is useful to improve production, transcription, expression, or safety in a subject. In certain embodiments, the engineered sequence is useful to increase efficacy of the resulting therapeutic compositions or treatment. In further embodiments, the engineered sequence is useful to increase the efficacy of the functional hGLA protein being expressed, and may also permit a lower dose of a therapeutic reagent that delivers the functional hGLA. In certain embodiments, the engineered hGLA coding sequence is characterized by an improved translation rate as compared to a wild type hGLA coding sequence.

By “engineered” is meant that the nucleic acid sequences encoding a functional hGLA enzyme described herein are assembled and placed into any suitable genetic element, e.g., naked DNA, phage, transposon, cosmid, episome, etc., which transfers the hGLA sequences carried thereon to a host cell, e.g., for generating non-viral delivery systems (e.g., RNA-based systems, naked DNA, or the like), or for generating viral vectors in a packaging host cell, and/or for delivery to a host cell in a subject. In certain embodiments, the genetic element is a vector. In one embodiment, the genetic element is a plasmid. The methods used to make such engineered constructs are known to those with skill in nucleic acid manipulation and include genetic engineering, recombinant engineering, and synthetic techniques. See, e.g., Green and Sambrook, Molecular Cloning: A Laboratory Manual, Cold Spring Harbor Press, Cold Spring Harbor, NY (2012).

The term “percent (%) identity”, “sequence identity”, “percent sequence identity”, or “percent identical” in the context of nucleic acid sequences refers to the residues in the two sequences which are the same when aligned for correspondence. The length of sequence identity comparison may be over the full-length of a construct, the full-length of a gene coding sequence, or a fragment of at least about 500 to 1000 nucleotides. However, identity among smaller fragments, for example, of at least about nine nucleotides, usually at least about 20 to 24 nucleotides, at least about 28 to 32 nucleotides, at least about 36 or more nucleotides, may also be desired.

Percent identity may be readily determined for amino acid sequences over the full-length of a protein, polypeptide, about 100 amino acids, about 300 amino acids, or a peptide fragment thereof or the corresponding nucleic acid sequence coding sequences. A suitable amino acid fragment may be at least about 8 amino acids in length, and may be up to about 50 amino acids. Generally, when referring to “identity”, “homology”, or “similarity” between two different sequences, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence.

Identity may be determined by preparing an alignment of sequences and through the use of a variety of algorithms and/or computer programs known in the art or commercially available (e.g., BLAST, ExPASy; Clustal Omega; FASTA; using, e.g., Needleman-Wunsch algorithm, Smith-Waterman algorithm). Alignments are performed using any of a variety of publicly or commercially available Multiple Sequence Alignment Programs. Sequence alignment programs are available for amino acid sequences, e.g., the “Clustal Omega”, “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

In certain embodiments, the hGLA coding sequence is less than 80% identical to the wild type hGLA sequence of SEQ ID NO: 1, and encodes the amino acid sequence of SEQ ID NO: 2, 7, or 17. In a further embodiment, the hGLA coding sequence comprises a sequence that is less than 80% identical to nucleotides (nt) 94 to 1287 of SEQ ID NO: 1, and encodes amino acids 32 to 429 of SEQ ID NO: 2, 7, or 17.

In certain embodiments the hGLA coding sequence shares less than about 99%, less than about 98%, less than about 97%, less than about 96%, less than about 95%, less than about 94%, less than about 93%, less than about 92%, less than about 91%, less than about 90%, less than about 89%, less than about 88%, less than about 87%, less than about 86%, less than about 85%, less than about 84%, less than about 83%, less than about 82%, less than about 81%, less than about 80%, less than about 79%, less than about 78%, less than about 77%, less than about 76%, less than about 75%, less than about 74%, less than about 73%, less than about 72%, less than about 71%, less than about 70%, less than about 69%, less than about 68%, less than about 67%, less than about 66%, less than about 65%, less than about 64%, less than about 63%, less than about 62%, less than about 61% or identity with the wild type hGLA coding sequence (SEQ ID NO: 1). In other embodiments, the hGLA coding sequence shares about 99%, about 98%, about 97%, about 96%, about 95%, about 94%, about 93%, about 92%, about 91%, about 90%, about 89%, about 88%, about 87%, about 86%, about 85%, about 84%, about 83%, about 82%, about 81%, about 80%, about 79%, about 78%, about 77%, about 76%, about 75%, about 74%, about 73%, about 72%, about 71%, about 70%, about 69%, about 68%, about 67%, about 66%, about 65%, about 64%, about 63%, about 62%, about 61% or less identity with the wild type hGLA coding sequence (SEQ ID NO: 1). In another embodiment, the hGLA coding sequence is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% identical to SEQ ID NO: 3, and the sequence encodes a functional hGLA. Identity may be with respect to a sequence that encodes a full-length hGLA (e.g., nt 1 to nt 1287 of SEQ ID NO: 1 or 3) or with respect to a sequence that encodes a mature hGLA (e.g., nt 94 to nt 1287 of SEQ ID NO: 1 or 3). In certain embodiments, the hGLA coding sequence includes nt 1 to 1287 of SEQ ID NO: 3, or a sequence at least 85%, 90%, 95%, or 99% identical thereto which encodes a full-length hGLA. In certain embodiments, the hGLA coding sequence includes the nt 94 to 1287 of SEQ ID NO: 3, or a sequence at least 85%, 90%, 95%, or 99% identical thereto encoding a functional hGLA.

In certain embodiments, an hGLA is provided which has amino substitutions at positions 233 and/or position 359, with reference to the numbering of the full-length native hGLA of SEQ ID NO: 2. In certain embodiments, the hGLA has a cysteine residue at position 233 and/or position 359. In certain embodiments, the hGLA comprises the amino acid sequence of SEQ ID NO: 7, or a sequence at least 95% identical thereto that has cysteine residue at position 233 and position 359. In other embodiments, the hGLA comprises amino acids 32 to 429 of SEQ ID NO:7, or a sequence at least 95% identical thereto that has cysteine residue at position 233 and position 359. In certain embodiment, an engineered coding sequence is provided that encodes the sequence of SEQ ID NO: 7, or a sequence at least 95% identical thereto that has cysteine residue at position 233 and position 359, wherein the coding sequence is shares less than about 99%, less than about 98%, less than about 97%, less than about 96%, less than about 95%, less than about 94%, less than about 93%, less than about 92%, less than about 91%, less than about 90%, less than about 89%, less than about 88%, less than about 87%, less than about 86%, less than about 85%, less than about 84%, less than about 83%, less than about 82%, less than about 81%, less than about 80%, less than about 79%, less than about 78%, less than about 77%, less than about 76%, less than about 75%, less than about 74%, less than about 73%, less than about 72%, less than about 71%, less than about 70%, less than about 69%, less than about 68%, less than about 67%, less than about 66%, less than about 65%, less than about 64%, less than about 63%, less than about 62%, less than about 61% or identity with the wild type hGLA coding sequence (SEQ ID NO: 1). In other embodiments, an engineered coding sequence is provided that encodes amino acids 32 to 429 of SEQ ID NO: 7, or a sequence at least 95% identical thereto that has cysteine residue at position 233 and position 359, wherein the coding sequence shares less than about 99%, less than about 98%, less than about 97%, less than about 96%, less than about 95%, less than about 94%, less than about 93%, less than about 92%, less than about 91%, less than about 90%, less than about 89%, less than about 88%, less than about 87%, less than about 86%, less than about 85%, less than about 84%, less than about 83%, less than about 82%, less than about 81%, less than about 80%, less than about 79%, less than about 78%, less than about 77%, less than about 76%, less than about 75%, less than about 74%, less than about 73%, less than about 72%, less than about 71%, less than about 70%, less than about 69%, less than about 68%, less than about 67%, less than about 66%, less than about 65%, less than about 64%, less than about 63%, less than about 62%, less than about 61% or identity with the wild type coding sequence for the mature hGLA (nt 94 to nt 1287 of SEQ ID NO: 1). In certain embodiments, an hGLA coding sequence is provided which comprises nt 94 to nt 1287 of SEQ ID NO: 4, or a sequence at least 85%, 90%, 95%, or 99% identical thereto, wherein the encoded functional hGLA has cysteine residues at position 233 and position 359. In certain embodiments, the hGLA coding sequence comprises nt 94 to 1287 of SEQ ID NO: 4. In a further embodiment, an hGLA coding sequence is provided which comprises SEQ ID NO: 4, or a sequence at least 85%, 90%, 95%, or 99% identical thereto, wherein the encoded functional hGLA has cysteine residues at position 233 and position 359. In certain embodiments, the hGLA coding sequence comprises SEQ ID NO: 4.

In certain embodiments, an hGLA is provided which has amino substitutions at positions 51 and/or position 360, with reference to the numbering of the full-length native hGLA of SEQ ID NO: 2). In certain embodiments, the hGLA has a cysteine residue at position 51 and/or position 360. In certain embodiments, the hGLA comprises the amino acid sequence of SEQ ID NO: 17, or a sequence at least 95% identical thereto that has cysteine residue at position 51 and position 360. In other embodiments, the hGLA comprises amino acids 32 to 429 of SEQ ID NO: 17, or a sequence at least 95% identical thereto that has cysteine residues at position 51 and position 360. In certain embodiments, an engineered coding sequence is provided that encodes the sequence of SEQ ID NO: 17, or a sequence at least 95% identical thereto that has cysteine residue at position 51 and position 360, wherein the sequence is shares less than about 99%, less than about 98%, less than about 97%, less than about 96%, less than about 95%, less than about 94%, less than about 93%, less than about 92%, less than about 91%, less than about 90%, less than about 89%, less than about 88%, less than about 87%, less than about 86%, less than about 85%, less than about 84%, less than about 83%, less than about 82%, less than about 81%, less than about 80%, less than about 79%, less than about 78%, less than about 77%, less than about 76%, less than about 75%, less than about 74%, less than about 73%, less than about 72%, less than about 71%, less than about 70%, less than about 69%, less than about 68%, less than about 67%, less than about 66%, less than about 65%, less than about 64%, less than about 63%, less than about 62%, less than about 61% or identity with the wild type hGLA coding sequence (SEQ ID NO: 1). In other embodiments, an engineered coding sequence is provided that encodes amino acids 32 to 429 of SEQ ID NO: 17, or a sequence at least 95% identical thereto that has cysteine residue at position 51 and position 360, wherein the sequence is shares less than about 99%, less than about 98%, less than about 97%, less than about 96%, less than about 95%, less than about 94%, less than about 93%, less than about 92%, less than about 91%, less than about 90%, less than about 89%, less than about 88%, less than about 87%, less than about 86%, less than about 85%, less than about 84%, less than about 83%, less than about 82%, less than about 81%, less than about 80%, less than about 79%, less than about 78%, less than about 77%, less than about 76%, less than about 75%, less than about 74%, less than about 73%, less than about 72%, less than about 71%, less than about 70%, less than about 69%, less than about 68%, less than about 67%, less than about 66%, less than about 65%, less than about 64%, less than about 63%, less than about 62%, less than about 61% or identity with the wild type coding sequence for the mature hGLA (94 to nt 1287 of SEQ ID NO: 1). In certain embodiments, an hGLA coding sequence is provided which comprises nt 94 to nt 1287 of SEQ ID NO: 5, or a sequence at least 85%, 90%, 95%, or 99% identical thereto, wherein the encoded functional hGLA has cysteine residues at position 51 and position 360. In certain embodiments, the hGLA coding sequence comprises nt 94 to nt 1287 of SEQ ID NO: 5. In a further embodiment, an hGLA coding sequence is provided which comprises SEQ ID NO: 5, or a sequence at least 85%, 90%, 95%, or 99% identical thereto, wherein the encoded functional hGLA has cysteine residues at position 51 and position 360. In certain embodiments, the hGLA coding sequence comprises SEQ ID NO: 5.

| | |
|---|---|
| hGLAco | atgcagctgagaaatcccgagctgcacctgggctgtgccctggctctgagatttctg |
| SEQ ID NO: 3 | gccctggtgtcttgggacatccctggcgctagagccctggataacggcctggcca |
| | gaacacctacaatgggctggctgcactgggagagattcatgtgcaacctggactg |
| | ccaagaggaacccgacagctgcatcagcgagaagctgttcatggaaatggccga |
| | gctgatggtgtccgaaggctggaaggacgccggctacgagtacctgtgcatcgac |
| | gactgttggatggcccctcagagagactctgagggcagactgcaggccgatcctc |
| | agagatttccccacggcattagacagctggccaactacgtgcacagcaagggcct |
| | gaagctgggcatctacgccgacgtgggcaacaagacctgtgccggctttcctggc |
| | agcttcggctactacgatatcgacgcccagaccttcgccgattggggagtcgatct |
| | gctgaagttcgacggctgctactgcgacagcctggaaaatctggccgacggctac |
| | aagcacatgtcactggccctgaatcggaccggccgcagcatcgtgtactcttgcga |
| | gtggcccctgtatatgtggcccttccagaagcctaactacaccgagatcagacagt |
| | actgcaaccactggcggaacttcgccgacatcgacgatagctggaagtccatcaa |
| | gagcatcctggactggaccagcttcaatcaagagcggatcgtggacgtggcagg |
| | acctggcggatggaacgatcctgacatgctggtcatcggcaacttcggcctgagct |
| | ggaaccagcaagtgacccagatggccctgtgggccattatggccgctcctctgttc |
| | atgagcaacgacctgagacacatcagccctcaggccaaggctctgctgcaggac |
| | aaggatgtgatcgctatcaaccaggatcctctgggcaagcagggctaccagctga |
| | gacagggcgacaatttcgaagtgtgggaaagacccctgagcggactggcttggg |
| | ccgtcgccatgatcaacagacaagagatcggcggaccccggtcctacacaattgc |
| | cgtggcttctctcggcaaaggcgtggcctgtaatcccgcctgctttatcacacagct |
| | gctgcccgtgaagagaaagctgggcttttacgagtggaccagcagactgcggag |
| | ccacatcaatcctaccggcacagtgctgctgcagctggaaaacacaatgcagatg |
| | agcctgaaggacctgctg |
| hGLAco(D233C_I359C) | atgcaactgagaaatcctgaactgcacctgggctgcgccctggctctgagatttctg |
| SEQ ID NO: 4 | gctctggtgtcctgggacatccctggcgctagagccctggataacggcctggcca |
| | gaacacctacaatgggctggctgcactgggagagattcatgtgcaacctggactg |
| | ccaagaggaacccgacagctgcatcagcgagaagctgttcatggaaatggccga |
| | gctgatggtgtccgaaggctggaaggacgccggctacgagtacctgtgcatcgac |
| | gactgttggatggcccctcagagagactctgagggcagactgcaggccgatcctc |
| | agagatttccccacggcattagacagctggccaactacgtgcacagcaagggcct |
| | gaagctgggcatctacgccgacgtgggcaacaagacctgtgccggctttcctggc |
| | agcttcggctactacgatatcgacgcccagaccttcgccgattggggagtcgatct |
| | gctgaagttcgacggctgctactgcgacagcctggaaaatctggccgacggctac |
| | aagcacatgtctctggccctgaatcggaccggcagatccatcgtgtacagctgcga |
| | gtggcccctgtacatgtggcccttccagaagcctaactacaccgagatcagacagt |
| | actgcaaccactggcggaacttcgccgacatctgcgatagctggaagtccatcaa |
| | gagcatcctggactggaccagcttcaatcaagagcggatcgtggacgtggcagg |
| | acctggcggatggaacgatcctgacatgctggtcatcggcaacttcggcctgagct |
| | ggaaccagcaagtgacccagatggccctgtgggccattatggccgctcctctgttc |
| | atgagcaacgacctgagacacatcagccctcaggccaaggctctgctgcaggac |
| | aaggatgtgatcgctatcaaccaggatcctctgggcaagcagggctaccagctga |
| | gacagggcgacaatttcgaagtgtgggaaagacccctgagcggactggcttggg |
| | ccgtcgccatgatcaaccggcaagagtgcggcggccccagatcctacacaatcg |
| | ccgtggccagtctcggcaaaggcgtggcatgtaatcccgcctgcttcatcacacag |
| | ctgctgcccgtgaagagaaagctgggcttttacgagtggaccagcagactgcgga |
| | gccacatcaatcctaccggcacagtgctgctgcagctggaaaacaccatgcagat |
| | gagcctgaaggacctgctg |
| hGLAco(M51C_G360C) | atgcaactgagaaatcctgaactgcacctgggctgcgccctggctctgagatttctg |
| SEQ ID NO: 5 | gctctggtgtcctgggacatccctggcgctagagccctggataacggcctggcca |
| | gaacacctacaatgggctggctgcactgggagagattctgctgcaacctggactg |
| | ccaagaggaacccgacagctgcatcagcgagaagctgttcatggaaatggccga |
| | gctgatggtgtccgaaggctggaaggacgccggctacgagtacctgtgcatcgac |
| | gactgttggatggcccctcagagagactctgagggcagactgcaggccgatcctc |
| | agagatttccccacggcattagacagctggccaactacgtgcacagcaagggcct |
| | gaagctgggcatctacgccgacgtgggcaacaagacctgtgccggctttcctggc |
| | agcttcggctactacgatatcgacgcccagaccttcgccgattggggagtcgatct |
| | gctgaagttcgacggctgctactgcgacagcctggaaaatctggccgacggctac |
| | aagcacatgtctctggccctgaatcggaccggcagatccatcgtgtacagctgcga |
| | gtggcccctgtacatgtggcccttccagaagcctaactacaccgagatcagacagt |
| | actgcaaccactggcggaacttcgccgacatcgacgatagctggaagtccatcaa |
| | gagcatcctggactggaccagcttcaatcaagagcggatcgtggacgtggcagg |
| | acctggcggatggaacgatcctgacatgctggtcatcggcaacttcggcctgagct |
| | ggaaccagcaagtgacccagatggccctgtgggccattatggccgctcctctgttc |
| | atgagcaacgacctgagacacatcagccctcaggccaaggctctgctgcaggac |
| | aaggatgtgatcgctatcaaccaggatcctctgggcaagcagggctaccagctga |
| | gacagggcgacaatttcgaagtgtgggaaagacccctgagcggactggcttggg |
| | ccgtcgccatgatcaaccggcaagagatttgcggccccagatcctacacaatcgc |
| | cgtggccagtctcggcaaaggcgtggcatgtaatcccgcctgcttcatcacacagc |
| | tgctgcccgtgaagagaaagctgggcttttacgagtggaccagcagactgcggag |
| | ccacatcaatcctaccggcacagtgctgctgcagctggaaaacaccatgcagatg |
| | agcctgaaggacctgctg |

As used herein, “a desired function” refers to an hGLA enzyme activity at least about 20%, about 25%, about 30%, about 35%, about 40%, about 45%, about 50%, about 55%, about 60%, about 65%, about 70%, about 75%, about 80%, about 85%, about 90%, about 95%, or about 100% of a healthy control.

As used herein, the phrases “ameliorate a symptom” and “improve a symptom”, and grammatical variants thereof, refer to reversal of a Fabry disease-related symptom, slowdown or prevention of progression of a Fabry disease-related symptom. In certain embodiments, the amelioration or improvement refers to the total number of symptoms in a patient after administration of the described composition(s) or use of the described method, which is reduced by about 5%, about 10%, about 20%, about 30%, about 40%, about 50%, about 60%, about 70%, about 80%, about 90%, about 95% compared to that before the administration or use. In another embodiment, the amelioration or improvement refers to the severity or progression of a symptom after administration of the described composition(s) or use of the described method, which is reduced by about 5%, about 10%, about 20%, about 30%, about 40%, about 50%, about 60%, about 70%, about 80%, about 90%, about 95% compared to that before the administration or use.

Still other hGLA variants may suitable. See also, International Patent Application No. PCT/US2019/05567, filed Oct. 10, 2019, which is incorporated by reference herein in its entirety.

It should be understood that the compositions in the functional hGLA or a hGLA coding sequences described herein are intended to be applied to other compositions, regimens, aspects, embodiments, and methods described across the Specification.

### 2. Expression Cassettes

In certain embodiments, provided herein are expression cassettes having an engineered nucleic acid sequence encoding a functional hGLA and a regulatory sequence which directs the expression thereof. In further embodiments, an expression cassette having an engineered nucleic acid sequence as described herein, which encodes a functional hGLA, and a regulatory sequence which directs the expression thereof.

As used herein, the term “expression” or “gene expression” refers to the process by which information from a gene is used in the synthesis of a functional gene product. The gene product may be a protein, a peptide, or a nucleic acid polymer (such as an RNA, a DNA or a PNA).

As used herein, an “expression cassette” refers to a nucleic acid polymer which comprises the coding sequences for a functional hGLA (including variants and fragments thereof) and a promoter. In further embodiments, the expression cassettes include one or more regulatory sequences in addition to a promoter. In certain embodiments, the expression vector is a vector genome. In certain embodiments, the expression cassette or vector genome is packaged into a vector. In certain embodiments, a plasmid that includes an expression cassette described herein is provided.

As used herein, the term “regulatory sequence” or “expression control sequence” refers to nucleic acid sequences, such as initiator sequences, enhancer sequences, and promoter sequences, which induce, repress, or otherwise control the transcription of protein encoding nucleic acid sequences to which they are operably linked.

As used herein, the term “operably linked” refers to both expression control sequences that are contiguous with the nucleic acid sequence encoding the hGLA and/or expression control sequences that act in trans or at a distance to control the transcription and expression thereof.

The term “heterologous” when used with reference to a protein or a nucleic acid in a plasmid, expression cassette, or vector, indicates that the protein or the nucleic acid is present with another sequence or subsequence which with which the protein or nucleic acid in question is not found in the same relationship to each other in nature.

In certain embodiments, the expression cassette provided includes a promoter that is a chicken β-actin promoter. A variety of chicken beta-actin promoters have been described alone, or in combination with various enhancer elements (e.g., CB7 is a chicken beta-actin promoter with cytomegalovirus enhancer elements, a CAG promoter, which includes the promoter, the first exon and first intron of chicken beta actin, and the splice acceptor of the rabbit beta-globin gene), a CBh promoter [S J Gray et al, Hu Gene Ther, 2011 September; 22(9): 1143-1153]. In other embodiments, a suitable promoter may include without limitation, an elongation factor 1 alpha (EF1 alpha) promoter (see, e.g., Kim D W et al, Use of the human elongation factor 1 alpha promoter as a versatile and efficient expression system. Gene. 1990 Jul. 16; 91(2):217-23), a Synapsin 1 promoter (see, e.g., Kugler S et al, Human synapsin 1 gene promoter confers highly neuron-specific long-term transgene expression from an adenoviral vector in the adult rat brain depending on the transduced area. Gene Ther. 2003 February; 10(4):337-47), a neuron-specific enolase (NSE) promoter (see, e.g., Kim J et al, Involvement of cholesterol-rich lipid rafts in interleukin-6-induced neuroendocrine differentiation of LNCaP prostate cancer cells. Endocrinology. 2004 February; 145(2):613-9. Epub 2003 Oct. 16), or a CB6 promoter (see, e.g., Large-Scale Production of Adeno-Associated Viral Vector Serotype-9 Carrying the Human Survival Motor Neuron Gene, Mol Biotechnol. 2016 January; 58(1):30-6. doi: 10.1007/s12033-015-9899-5).

Examples of promoters that are tissue-specific are well known for liver and other tissues (albumin, Miyatake et al., (1997) J. Virol., 71:5124-32; hepatitis B virus core promoter, Sandig et al., (1996) Gene Ther., 3:1002-9; alpha-fetoprotein (AFP), Arbuthnot et al., (1996) Hum. Gene Ther., 7:1503-14), bone osteocalcin (Stein et al., (1997) Mol. Biol. Rep., 24:185-96); bone sialoprotein (Chen et al., (1996) J. Bone Miner. Res., 11:654-64), lymphocytes (CD2, Hansal et al., (1998) J. Immunol., 161:1063-8; immunoglobulin heavy chain; T cell receptor chain), neuronal such as neuron-specific enolase (NSE) promoter (Andersen et al., (1993) Cell. Mol. Neurobiol., 13:503-15), neurofilament light-chain gene (Piccioli et al., (1991) Proc. Natl. Acad. Sci. USA, 88:5611-5), and the neuron-specific vgf gene (Piccioli et al., (1995) Neuron, 15:373-84), among others. In certain embodiments, the promoter is a human thyroxine binding globulin (TBG) promoter. Alternatively, a regulatable promoter may be selected. See, e.g., WO 2011/126808B2, incorporated by reference herein.

In certain embodiments, the expression cassette includes one or more expression enhancers. In certain embodiment, the expression cassette contains two or more expression enhancers. These enhancers may be the same or may be different. For example, an enhancer may include an Alpha mic/bik enhancer or a CMV enhancer. This enhancer may be present in two copies which are located adjacent to one another. Alternatively, the dual copies of the enhancer may be separated by one or more sequences. In still further embodiments, the expression cassette further contains an intron, e.g., a chicken beta-actin intron, a human (3-globulin intron, SV40 intron, and/or a commercially available Promega® intron. Other suitable introns include those known in the art, e.g., such as are described in WO 2011/126808.

The expression cassettes provided may include one or more expression enhancers such as post-transcriptional regulatory element from hepatitis viruses of woodchuck (WPRE), human (HPRE), ground squirrel (GPRE) or arctic ground squirrel (AGSPRE); or a synthetic post-transcriptional regulatory element. These expression-enhancing elements are particularly advantageous when placed in a 3′ UTR and can significantly increase mRNA stability and/or protein yield. In certain embodiments, the expressions cassettes provided include a regulator sequence that is a woodchuck hepatitis virus posttranscriptional regulatory element (WPRE) or a variant thereof. Suitable WPRE sequences are provided in the vector genomes described herein and are known in the art (e.g., such as those are described in U.S. Pat. Nos. 6,136,597, 6,287,814, and 7,419,829, which are incorporated by reference). In certain embodiments, the WPRE is a variant that has been mutated to eliminate expression of the woodchuck hepatitis B virus X (WHX) protein, including, for example, mutations in the start codon of the WHX gene (See, Zanta-Boussif et al., Gene Ther. 2009 May; 16(5):605-19, which is incorporated by reference). In certain embodiments, the WPRE comprises the nucleotide sequence provided in SEQ ID NO: 27. In other embodiments, enhancers are selected from a non-viral source

Further, expression cassettes provided include a suitable polyadenylation signal. In certain embodiments, the polyA sequence is a rabbit β-globin poly A. See, e.g., WO 2014/151341. In another embodiments, the polyA sequence is a bovine growth hormone polyA. Alternatively, another polyA, e.g., a human growth hormone (hGH) polyadenylation sequence, an S450 polyA, or a synthetic polyA is included.

In certain embodiments, the expression cassette may include one or more miRNA (also referred to as miR or micro-RNA) target sequences in the untranslated region(s). The miRNA target sequences are designed to be specifically recognized by miRNA present in cells in which transgene expression is undesirable and/or reduced levels of transgene expression are desired. In certain embodiments, the expression cassette includes miRNA target sequences that specifically reduce expression of hGLA in dorsal root ganglion. In certain embodiments, the miRNA target sequences are located in the 3′ UTR, 5′ UTR, and/or in both 3′ and 5′ UTR of an expression cassette. In certain embodiments, the expression cassette comprises at least two tandem repeats of dorsal root ganglion (DRG)-specific miRNA target sequences, wherein the at least two tandem repeats comprise at least a first miRNA target sequence and at least a second miRNA target sequence which may be the same or different. In certain embodiments, the start of the first of the at least two drg-specific miRNA tandem repeats is within 20 nucleotides from the 3′ end of the hGLA-coding sequence. In certain embodiments, the start of the first of the at least two DRG-specific miRNA tandem repeats is at least 100 nucleotides from the 3′ end of the hGLA-coding sequence. In certain embodiments, the miRNA tandem repeats comprise 200 to 1200 nucleotides in length. In certain embodiment, the inclusion of miR targets does not modify the expression or efficacy of the therapeutic transgene in one or more target tissues, relative to the expression cassette lacking the miR target sequences.

In certain embodiments, the expression cassette contains at least one miRNA target sequence that is a miR-183 target sequence. In certain embodiments, the expression cassette contains a miR-183 target sequence that includes AGTGAATTCTACCAGTGCCATA (SEQ ID NO: 31), where the sequence complementary to the miR-183 seed sequence is underlined. In certain embodiments, the expression cassette contains more than one copy (e.g. two or three copies) of a sequence that is 100% complementary to the miR-183 seed sequence. In certain embodiments, a miR-183 target sequence is about 7 nucleotides to about 28 nucleotides in length and includes at least one region that is at least 100% complementary to the miR-183 seed sequence. In certain embodiments, a miR-183 target sequence contains a sequence with partial complementarity to SEQ ID NO: 31 and, thus, when aligned to SEQ ID NO: 31, there are one or more mismatches. In certain embodiments, a miR-183 target sequence comprises a sequence having at least 1, 2, 3, 4, 5, 6, 7, 8, 9, or 10 mismatches when aligned to SEQ ID NO: 31, where the mismatches may be non-contiguous. In certain embodiments, a miR-183 target sequence includes a region of 100% complementarity which also comprises at least 30% of the length of the miR-183 target sequence. In certain embodiments, the region of 100% complementarity includes a sequence with 100% complementarity to the miR-183 seed sequence. In certain embodiments, the remainder of a miR-183 target sequence has at least about 80% to about 99% complementarity to miR-183. In certain embodiments, the expression cassette includes a miR-183 target sequence that comprises a truncated SEQ ID NO: 31, i.e., a sequence that lacks at least 1, 2, 3, 4, 5, 6, 7, 8, 9, or 10 nucleotides at either or both the 5′ or 3′ ends of SEQ ID NO: 31. In certain embodiments, the expression cassette comprises a transgene and one miR-183 target sequence. In yet other embodiments, the expression cassette comprises at least two, three or four miR-183 target sequences. In certain embodiments, the inclusion of at two, three or four miR-183 target sequences in the expression cassette results in increased levels of transgene expression in a target tissue, such as the heart.

In certain embodiments, the expression cassette contains at least one miRNA target sequence that is a miR-182 target sequence. In certain embodiments, the expression cassette contains an miR-182 target sequence that includes AGTGTGAGTTCTACCATTGCCAAA (SEQ ID NO: 32). In certain embodiments, the expression cassette contains more than one copy (e.g. two or three copies) of a sequence that is 100% complementary to the miR-182 seed sequence. In certain embodiments, a miR-182 target sequence is about 7 nucleotides to about 28 nucleotides in length and includes at least one region that is at least 100% complementary to the miR-182 seed sequence. In certain embodiments, a miR-182 target sequence contains a sequence with partial complementarity to SEQ ID NO: 32 and, thus, when aligned to SEQ ID NO: 32, there are one or more mismatches. In certain embodiments, a miR-183 target sequence comprises a sequence having at least 1, 2, 3, 4, 5, 6, 7, 8, 9, or 10 mismatches when aligned to SEQ ID NO: 32, where the mismatches may be non-contiguous. In certain embodiments, a miR-182 target sequence includes a region of 100% complementarity which also comprises at least 30% of the length of the miR-182 target sequence. In certain embodiments, the region of 100% complementarity includes a sequence with 100% complementarity to the miR-182 seed sequence. In certain embodiments, the remainder of a miR-182 target sequence has at least about 80% to about 99% complementarity to miR-182. In certain embodiments, the expression cassette includes a miR-182 target sequence that comprises a truncated SEQ ID NO: 32, i.e., a sequence that lacks at least 1, 2, 3, 4, 5, 6, 7, 8, 9, or 10 nucleotides at either or both the 5′ or 3′ ends of SEQ ID NO: 32. In certain embodiments, the expression cassette comprises a transgene and one miR-182 target sequence. In yet other embodiments, the expression cassette comprises at least two, three or four miR-182 target sequences.

The term “tandem repeats” is used herein to refer to the presence of two or more consecutive miRNA target sequences. These miRNA target sequences may be continuous, i.e., located directly after one another such that the 3′ end of one is directly upstream of the 5′ end of the next with no intervening sequences, or vice versa. In another embodiment, two or more of the miRNA target sequences are separated by a short spacer sequence.

As used herein, as “spacer” is any selected nucleic acid sequence, e.g., of 1, 2, 3, 4, 5, 6, 7, 8, 9 or 10 nucleotides in length which is located between two or more consecutive miRNA target sequences. In certain embodiments, the spacer is 1 to 8 nucleotides in length, 2 to 7 nucleotides in length, 3 to 6 nucleotides in length, four nucleotides in length, 4 to 9 nucleotides, 3 to 7 nucleotides, or values which are longer. Suitably, a spacer is a non-coding sequence. In certain embodiments, the spacer may be of four (4) nucleotides. In certain embodiments, the spacer is GGAT. In certain embodiments, the spacer is six (6) nucleotides. In certain embodiments, the spacer is CACGTG or GCATGC.

In certain embodiments, the tandem repeats contain two, three, four or more of the same miRNA target sequence. In certain embodiments, the tandem repeats contain at least two different miRNA target sequences, at least three different miRNA target sequences, or at least four different miRNA target sequences, etc. In certain embodiments, the tandem repeats may contain two or three of the same miRNA target sequence and a fourth miRNA target sequence which is different.

In certain embodiments, there may be at least two different sets of tandem repeats in the expression cassette. For example, a 3′ UTR may contain a tandem repeat immediately downstream of the transgene, UTR sequences, and two or more tandem repeats closer to the 3′ end of the UTR. In another example, the 5′ UTR may contain one, two or more miRNA target sequences. In another example the 3′ UTR may contain tandem repeats and the 5′ UTR may contain at least one miRNA target sequence.

In certain embodiments, the expression cassette contains two, three, four or more tandem repeats which start within about 0 to 20 nucleotides of the stop codon for the transgene. In other embodiments, the expression cassette contains the miRNA tandem repeats at least 100 to about 4000 nucleotides from the stop codon for the transgene.

See also International Patent Application No. PCT/US19/67872, filed Dec. 20, 2019, and International Patent Application No. PCT/US21/32003, filed May 12, 2021, which are incorporated by reference in their entireties.

It should be understood that the compositions in the expression cassettes described are intended to be applied to other compositions, regimens, aspects, embodiments and methods described across the Specification.

### 3. Vector

In one aspect, provided herein is a vector comprising a nucleic acid sequence encoding a functional hGLA. In certain embodiments, the vector comprises an expression cassette as described herein for delivery of a hGLA coding sequence.

A “vector” as used herein is a biological or chemical moiety comprising a nucleic acid sequence which can be introduced into an appropriate target cell for replication or expression of said nucleic acid sequence. Examples of a vector include but not limited to a recombinant virus, a plasmid, Lipoplexes, a Polymersome, Polyplexes, a dendrimer, a cell penetrating peptide (CPP) conjugate, a magnetic particle, or a nanoparticle. In certain embodiments, a vector is a nucleic acid molecule into which an engineered nucleic acid encoding a functional hGLA may be inserted, which can then be introduced into an appropriate target cell. Such vectors preferably have one or more origin of replication, and one or more site into which the recombinant DNA can be inserted. Vectors often have means by which cells with vectors can be selected from those without, e.g., they encode drug resistance genes. Common vectors include plasmids, viral genomes, and “artificial chromosomes”. Conventional methods of generation, production, characterization or quantification of the vectors are available to one of skill in the art.

In certain embodiments, the vector is a non-viral plasmid that comprises an expression cassette described herein (for example, “naked DNA”, “naked plasmid DNA”, RNA, and mRNA, which may be coupled with various compositions and nano particles, including, for examples, micelles, liposomes, cationic lipid-nucleic acid compositions, poly-glycan compositions and other polymers, lipid and/or cholesterol-based-nucleic acid conjugates) and other constructs such as are described herein. See, e.g., X. Su et al, Mol. Pharmaceutics, 2011, 8 (3), pp 774-787; web publication: Mar. 21, 2011; WO2013/182683, WO 2010/053572 and WO 2012/170930, all of which are incorporated herein by reference.

In certain embodiments, the vector described herein is a “replication-defective virus” or a “viral vector” which refers to a synthetic or artificial viral particle in which an expression cassette containing a nucleic acid sequence encoding hGLA is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless”-containing only the nucleic acid sequence encoding hGLA flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication.

As used herein, a recombinant virus vector is an adeno-associated virus (AAV), an adenovirus, a bocavirus, a hybrid AAV/bocavirus, a herpes simplex virus, or a lentivirus.

In certain embodiments, a host cell having a nucleic acid including an hGLA-coding sequence is provided. In certain embodiments, the host cell contains a plasmid having an hGLA-coding sequence as described herein.

As used herein, the term “host cell” may refer to the packaging cell line in which a vector (e.g., a recombinant AAV) is produced. A host cell may be a prokaryotic or eukaryotic cell (e.g., human, insect, or yeast) that contains exogenous or heterologous DNA that has been introduced into the cell by any means, e.g., electroporation, calcium phosphate precipitation, microinjection, transformation, viral infection, transfection, liposome delivery, membrane fusion techniques, high velocity DNA-coated pellets, viral infection and protoplast fusion. Examples of host cells may include, but are not limited to an isolated cell, a cell culture, an Escherichia coli cell, a yeast cell, a human cell, a non-human cell, a mammalian cell, a non-mammalian cell, an insect cell, an HEK-293 cell, a liver cell, a kidney cell, a cell of the central nervous system, a neuron, a glial cell, or a stem cell.

In certain embodiments, a host cell contains an expression cassette for production of hGLA such that the protein is produced in sufficient quantities in vitro for isolation or purification. In certain embodiments, the host cell contains an expression cassette encoding hGLA (including, for example, a functional fragment thereof). As provided herein, hGLA polypeptide may be included in a pharmaceutical composition administered to a subject as a therapeutic (i.e., enzyme replacement therapy).

As used herein, the term “target cell” refers to any cell in which expression of the functional hGLA is desired. In certain embodiments, the term “target cell” is intended to reference the cells of the subject being treated for Fabry disease. Examples of target cells may include, but are not limited to, liver cells, kidney cells, smooth muscle cells, and neurons. In certain embodiments, the vector is delivered to a target cell ex vivo. In certain embodiments, the vector is delivered to the target cell in vivo.

It should be understood that the compositions in the vector described herein are intended to be applied to other compositions, regimens, aspects, embodiments, and methods described across the Specification.

4. Recombinant Adeno-Associated Virus (rAAV)

In certain embodiments, provided herein is a rAAV comprising an AAV capsid and a vector genome packaged therein. The vector genome comprises an AAV 5′ inverted terminal repeat (ITR), a nucleic acid sequence encoding a functional hGLA as described herein, a regulatory sequence which directs expression of hGLA in a target cell, and an AAV 3′ ITR.

In certain embodiments, the vector genome comprises an expression cassette as provided herein flanked by an AAV 5′ ITR and an AAV 3′ ITR. Such rAAV are suitable for use in the treatment of Fabry disease.

As used herein, a “rAAV.hGLA” refers to a rAAV having a vector genome that includes an hGLA coding sequence. A “rAAVhu68.hGLA” refers to rAAV having an AAVhu68 capsid and a vector genome that includes an hGLA coding sequence.

As used herein, a “vector genome” refers to a nucleic acid sequence packaged inside a vector. In one embodiment, the vector genome refers to the nucleic acid sequence packaged inside a rAAV capsid forming an rAAV vector. Such a nucleic acid sequence contains AAV inverted terminal repeat sequences (ITRs). In certain embodiments, the ITRs are from an AAV different than that supplying a capsid. In a preferred embodiment, the ITR sequences from AAV2, or the deleted version thereof (AITR), which may be used for convenience and to accelerate regulatory approval. However, ITRs from other AAV sources may be selected. Where the source of the ITRs is from AAV2 and the AAV capsid is from another AAV source, the resulting vector may be termed pseudotyped. Typically, AAV vector genome comprises an AAV 5′ ITR, regulatory sequence(s), an hGLA coding sequence, and an AAV 3′ ITR. However, other configurations of these elements may be suitable. A shortened version of the 5′ ITR, termed AITR, has been described in which the D-sequence and terminal resolution site (trs) are deleted. In certain embodiments, the vector genome includes a shortened AAV2 ITR of 130 base pairs, wherein the external A elements is deleted. The shortened ITR is reverted back to the wild type length of 145 base pairs during vector DNA amplification using the internal A element as a template. In other embodiments, the full-length AAV 5′ and 3′ ITRs are used. In certain embodiments, the vector genome includes one or more miRNA target sequences.

In certain embodiments, a rAAV is provided having a vector genome that includes an AAV 5′ ITR, a promoter, an hGLA coding sequence, a poly A sequence, and an AAV 3′ ITR. In certain embodiments, a rAAV is provided having a vector genome that includes an AAV 5′ ITR, a promoter, an intron, an hGLA coding sequence, a poly A sequence, and an AAV 3′ ITR. In certain embodiments, a rAAV is provided having a vector genome that includes an AAV 5′ ITR, a promoter, an hGLA coding sequence, a WPRE, a poly A sequence, and an AAV 3′ ITR. In certain embodiments, a rAAV is provided having a vector genome that includes an AAV 5′ ITR, a promoter, an intron, an hGLA coding sequence, a WPRE, a poly A sequence, and an AAV 3′ ITR. In certain embodiments, the vector genome has an enhancer from a non-viral source in place of the WPRE element.

In certain embodiments, a rAAV is provided having a vector genome that includes an AAV 5′ ITR, a promoter, a chicken beta-actin intron, an hGLA coding sequence, a WPRE, a poly A sequence, and an AAV 3′ ITR. In certain embodiments, a rAAV is provided having a vector genome that includes an AAV 5′ ITR, a CB7 promoter, a chicken beta-actin intron, an hGLA coding sequence, a WPRE, a rabbit beta globin poly A sequence, and an AAV 3′ ITR. In certain embodiments, a rAAV is provided having a vector genome that includes an AAV 5′ ITR, a TBG promoter, a chicken beta-actin intron, an hGLA coding sequence, a WPRE, a bovine growth hormone poly A sequence, and an AAV 3′ ITR. In certain embodiments, a rAAV is provided having a vector genome that includes an AAV 5′ ITR, a TBG promoter, an SV40 intron, an hGLA coding sequence, a WPRE, a bovine growth hormone poly A sequence, and an AAV3′ ITR. In certain embodiments, the vector genome has an enhancer from a non-viral source in place of the WPRE element.

In certain embodiments, a rAAV is provided having a vector genome that includes an AAV 5′ ITR, a promoter, a chicken beta-actin intron, an hGLA coding sequence, a poly A sequence, and an AAV 3′ ITR. In certain embodiments, a rAAV is provided having a vector genome that includes an AAV 5′ ITR, a CB7 promoter, a chicken beta-actin intron, an hGLA coding sequence, a rabbit globin poly A sequence, and an AAV 3′ ITR. In certain embodiments, a rAAV is provided having a vector genome that includes an AAV 5′ ITR, a TBG promoter, a chicken beta-actin intron, an hGLA coding sequence, a bovine growth hormone poly A sequence, and an AAV 3′ ITR. In certain embodiments, a rAAV is provided having a vector genome that includes an AAV 5′ ITR, a TBG promoter, an SV40 intron, an hGLA coding sequence, a bovine growth hormone poly A sequence, and an AAV 3′ ITR.

In one embodiment, a rAAV is provided having a vector genome set forth in SEQ ID NO: 6, 8, 10, 12, 14, 16, or 18, or a sequence at least 85% identical thereto.

As used herein, the terms “rAAV” and “artificial AAV” used interchangeably, mean, without limitation, an AAV comprising a capsid protein and a vector genome packaged therein, wherein the vector genome comprising a nucleic acid heterologous to the AAV. In one embodiment, the capsid protein is a non-naturally occurring capsid. Such an artificial capsid may be generated by any suitable technique, using a selected AAV sequence (e.g., a fragment of a vp1 capsid protein) in combination with heterologous sequences which may be obtained from a different selected AAV, non-contiguous portions of the same AAV, from a non-AAV viral source, or from a non-viral source. An artificial AAV may be, without limitation, a pseudotyped AAV, a chimeric AAV capsid, a recombinant AAV capsid, or a “humanized” AAV capsid. Pseudotyped vectors, wherein the capsid of one AAV is replaced with a heterologous capsid protein, are useful in the invention. In one embodiment, AAV2/5 and AAV2/8 are exemplary pseudotyped vectors. The selected genetic element may be delivered by any suitable method, including transfection, electroporation, liposome delivery, membrane fusion techniques, high velocity DNA-coated pellets, viral infection and protoplast fusion. The methods used to make such constructs are known to those with skill in nucleic acid manipulation and include genetic engineering, recombinant engineering, and synthetic techniques. See, e.g., Green and Sambrook, Molecular Cloning: A Laboratory Manual, Cold Spring Harbor Press, Cold Spring Harbor, NY (2012).

The term “AAV” as used herein refers to naturally occurring adeno-associated viruses, adeno-associated viruses available to one of skill in the art and/or in light of the composition(s) and method(s) described herein, as well as artificial AAVs. An adeno-associated virus (AAV) viral vector is an AAV DNase-resistant particle having an AAV protein capsid into which is packaged expression cassette flanked by AAV inverted terminal repeat sequences (ITRs) for delivery to target cells. An AAV capsid is composed of 60 capsid (cap) protein subunits, VP1, VP2, and VP3, that are arranged in an icosahedral symmetry in a ratio of approximately 1:1:10 to 1:1:20, depending upon the selected AAV. Various AAVs may be selected as sources for capsids of AAV viral vectors as identified above. See, e.g., US Published Patent Application No. 2007-0036760-A1; US Published Patent Application No. 2009-0197338-A1; EP 1310571. See also, WO 2003/042397 (AAV7 and other simian AAV), U.S. Pat. Nos. 7,790,449 and 7,282,199 (AAV8), WO 2005/033321 and U.S. Pat. No. 7,906,111 (AAV9), and WO 2006/110689, and WO 2003/042397 (rh.10). These documents also describe other AAV which may be selected for generating AAV and are incorporated by reference. Among the AAVs isolated or engineered from human or non-human primates (NHP) and well characterized, human AAV2 is the first AAV that was developed as a gene transfer vector; it has been widely used for efficient gene transfer experiments in different target tissues and animal models. Unless otherwise specified, the AAV capsid, ITRs, and other selected AAV components described herein, may be readily selected from among any AAV, including, without limitation, the AAVs commonly identified as AAV1, AAV2, AAV3, AAV4, AAV5, AAV6, AAV7, AAV8, AAV9, AAV8 bp, AAV7M8 and AAVAnc80, AAVhu68, and variants of any of the known or mentioned AAVs or AAVs yet to be discovered or variants or mixtures thereof. An AAV9 capsid includes an rAAV having capsid proteins comprising an amino acid sequence which is 99% identical to AAS99264. See, also U.S. Pat. No. 7,906,111 and WO 2005/033321. rAAVs having a AVVhu68 capsid are described in, for example, WO 2018/160582, which is incorporated herein by reference. In certain embodiments, the capsid protein is designated by a number or a combination of numbers and letters following the term “AAV” in the name of the rAAV vector. See also PCT/US19/19804 and PCT/US19/19861, each entitled “Novel Adeno-Associated Virus (AAV) Vectors, AAV Vectors Having Reduced Capsid Deamidation And Uses Therefor” and filed Feb. 27, 2019, which are incorporated by reference herein in their entireties.

As used herein, relating to AAV, the term “variant” means any AAV sequence which is derived from a known AAV sequence, including those with a conservative amino acid replacement, and those sharing at least 70%, at least 75%, at least 80%, at least 85%, at least 90%, at least 95%, at least 97%, at least 99% or greater sequence identity over the amino acid or nucleic acid sequence. In another embodiment, the AAV capsid includes variants which may include up to about 10% variation from any described or known AAV capsid sequence. That is, the AAV capsid shares about 90% identity to about 99.9% identity, about 95% to about 99% identity or about 97% to about 98% identity to an AAV capsid provided herein and/or known in the art. In one embodiment, the AAV capsid shares at least 95% identity with an AAV capsid. When determining the percent identity of an AAV capsid, the comparison may be made over any of the variable proteins (e.g., vp1, vp2, or vp3). As used herein “AAV9 variants” include those described in, e.g., WO2016/049230, U.S. Pat. No. 8,927,514, US 2015/0344911, and U.S. Pat. No. 8,734,809.

In certain embodiments, the AAV capsid is selected from among natural and engineered clade F adeno-associated viruses. In certain embodiments, the rAAV provided herein comprises an AAVhu68 capsid. AAVhu68 is within clade F. AAVhu68 (SEQ ID NO: 21) varies from another Clade F virus AAV9 by two encoded amino acids at positions 67 and 157 of vp1. In contrast, other Clade F AAVs (AAV9, hu31, hu32) have an Ala at position 67 and an Ala at position 157. However, in other embodiments, an AAV capsid is selected from a different clade, e.g., clade A, B, C, D, or E, or from an AAV source outside of any of these clades.

A rAAVhu68 includes an AAVhu68 capsid and a vector genome. In one embodiment, a composition comprising rAAVhu68 comprises an assembly of a heterogeneous population of vp1, a heterogeneous population of vp2, and a heterogeneous population of vp3 proteins. As used herein when used to refer to vp capsid proteins, the term “heterogeneous” or any grammatical variation thereof, refers to a population consisting of elements that are not the same, for example, having vp1, vp2 or vp3 monomers (proteins) with different modified amino acid sequences. SEQ ID NO: 21 provides the encoded amino acid sequence of the AAVhu68 vp1 protein. The AAVhu68 capsid contains subpopulations within the vp1 proteins, within the vp2 proteins and within the vp3 proteins which have modifications from the predicted amino acid residues in SEQ ID NO: 21. These subpopulations include, at a minimum, certain deamidated asparagine (N or Asn) residues. For example, certain subpopulations comprise at least one, two, three or four highly deamidated asparagines (N) positions in asparagine-glycine pairs in SEQ ID NO: 21 and optionally further comprising other deamidated amino acids, wherein the deamidation results in an amino acid change and other optional modifications. The various combinations of these and other modifications are described herein.

As used herein, a “subpopulation” of vp proteins refers to a group of vp proteins which has at least one defined characteristic in common and which consists of at least one group member to less than all members of the reference group, unless otherwise specified. For example, a “subpopulation” of vp1 proteins is at least one (1) vp1 protein and less than all vp1 proteins in an assembled AAV capsid, unless otherwise specified. A “subpopulation” of vp3 proteins may be one (1) vp3 protein to less than all vp3 proteins in an assembled AAV capsid, unless otherwise specified. For example, vp1 proteins may be a subpopulation of vp proteins; vp2 proteins may be a separate subpopulation of vp proteins, and vp3 are yet a further subpopulation of vp proteins in an assembled AAV capsid. In another example, vp1, vp2 and vp3 proteins may contain subpopulations having different modifications, e.g., at least one, two, three or four highly deamidated asparagines, e.g., at asparagine-glycine pairs. Unless otherwise specified, highly deamidated refers to at least 45% deamidated, at least 50% deamidated, at least 60% deamidated, at least 65% deamidated, at least 70%, at least 75%, at least 80%, at least 85%, at least 90%, at least 95%, at least 97%, at least 99%, up to about 100% deamidated at a referenced amino acid position, as compared to the predicted amino acid sequence at the reference amino acid position (e.g., at least 80% of the asparagines at amino acid 57 of SEQ ID NO: 21 may be deamidated based on the total vp1 proteins or 20% of the asparagines at amino acid 409 of SEQ ID NO: 21 may be deamidated based on the total vp1, vp2 and vp3 proteins). Such percentages may be determined using 2D-gel, mass spectrometry techniques, or other suitable techniques.

As provided herein, each deamidated N of SEQ ID NO: 21 may independently be aspartic acid (Asp), isoaspartic acid (isoAsp), aspartate, and/or an interconverting blend of Asp and isoAsp, or combinations thereof. Any suitable ratio of α- and isoaspartic acid may be present. For example, in certain embodiments, the ratio may be from 10:1 to 1:10 aspartic to isoaspartic, about 50:50 aspartic: isoaspartic, or about 1:3 aspartic: isoaspartic, or another selected ratio. In certain embodiments, one or more glutamine (Q) in SEQ ID NO: 21 deamidates to glutamic acid (Glu), i.e., α-glutamic acid, γ-glutamic acid (Glu), or a blend of α- and γ-glutamic acid, which may interconvert through a common glutarinimide intermediate. Any suitable ratio of α- and γ-glutamic acid may be present. For example, in certain embodiments, the ratio may be from 10:1 to 1:10 α to γ, about 50:50 α:γ, or about 1:3 α:γ, or another selected ratio.

Thus, an rAAVhu68 includes subpopulations within the rAAVhu68 capsid of vp1, vp2 and/or vp3 proteins with deamidated amino acids, including at a minimum, at least one subpopulation comprising at least one highly deamidated asparagine. In addition, other modifications may include isomerization, particularly at selected aspartic acid (D or Asp) residue positions. In still other embodiments, modifications may include an amidation at an Asp position.

In certain embodiments, an AAVhu68 capsid contains subpopulations of vp1, vp2 and vp3 having at least 4 to at least about 25 deamidated amino acid residue positions, of which at least 1 to 10% are deamidated as compared to the encoded amino acid sequence of SEQ ID NO: 21. The majority of these may be N residues. However, Q residues may also be deamidated.

In certain embodiments, an AAVhu68 capsid is further characterized by one or more of the following. AAVhu68 capsid proteins that comprise: AAVhu68 vp1 proteins produced by expression from a nucleic acid sequence which encodes the predicted amino acid sequence of 1 to 736 of SEQ ID NO: 21, vp1 proteins produced from SEQ ID NO: 20, or vp1 proteins produced from a nucleic acid sequence at least 70% identical to SEQ ID NO: 20 which encodes the predicted amino acid sequence of 1 to 736 of SEQ ID NO: 23; AAVhu68 vp2 proteins produced by expression from a nucleic acid sequence which encodes the predicted amino acid sequence of at least about amino acids 138 to 736 of SEQ ID NO: 21, vp2 proteins produced from a sequence comprising at least nucleotides 412 to 2211 of SEQ ID NO: 20, or vp2 proteins produced from a nucleic acid sequence at least 70% identical to at least nucleotides 412 to 2211 of SEQ ID NO: 20 which encodes the predicted amino acid sequence of at least about amino acids 138 to 736 of SEQ ID NO: 21, and/or AAVhu68 vp3 proteins produced by expression from a nucleic acid sequence which encodes the predicted amino acid sequence of at least about amino acids 203 to 736 of SEQ ID NO: 21, vp3 proteins produced from a sequence comprising at least nucleotides 607 to 2211 of SEQ ID NO: 20, or vp3 proteins produced from a nucleic acid sequence at least 70% identical to at least nucleotides 607 to 2211 of SEQ ID NO: 20 which encodes the predicted amino acid sequence of at least about amino acids 203 to 736 of SEQ ID NO: 21.

Additionally or alternatively, an AAV capsid is provided which comprises a heterogeneous population of vp1 proteins optionally comprising a valine at position 157, a heterogeneous population of vp2 proteins optionally comprising a valine at position 157, and a heterogeneous population of vp3 proteins, wherein at least a subpopulation of the vp1 and vp2 proteins comprise a valine at position 157 and optionally further comprising a glutamic acid at position 67 based on the numbering of the vp1 capsid of SEQ ID NO: 21. Additionally or alternatively, an AAVhu68 capsid is provided which comprises a heterogeneous population of vp1 proteins which are the product of a nucleic acid sequence encoding the amino acid sequence of SEQ ID NO: 21, a heterogeneous population of vp2 proteins which are the product of a nucleic acid sequence encoding the amino acid sequence of at least about amino acids 138 to 736 of SEQ ID NO: 21, and a heterogeneous population of vp3 proteins which are the product of a nucleic acid sequence encoding at least amino acids 203 to 736 of SEQ ID NO: 21, wherein: the vp1, vp2 and vp3 proteins contain subpopulations with amino acid modifications

The AAVhu68 vp1, vp2 and vp3 proteins are typically expressed as alternative splice variants encoded by the same nucleic acid sequence which encodes the full-length vp1 amino acid sequence of SEQ ID NO: 21 (amino acid 1 to 736). Optionally the vp1-encoding sequence is used alone to express the vp1, vp2 and vp3 proteins. Alternatively, this sequence may be co-expressed with one or more of a nucleic acid sequence which encodes the AAVhu68 vp3 amino acid sequence of SEQ ID NO: 21 (about aa 203 to 736) without the vp1-unique region (about aa 1 to about aa 137) and/or vp2-unique regions (about aa 1 to about aa 202), or a strand complementary thereto, the corresponding mRNA (about nt 607 to about nt 2211 of SEQ ID NO: 20), or a sequence at least 70% to at least 99% (e.g., at least 85%, at least 90%, at least 95%, at least 97%, at least 98% or at least 99%) identical to SEQ ID NO: 20 which encodes aa 203 to 736 of SEQ ID NO: 21. Additionally, or alternatively, the vp1-encoding and/or the vp2-encoding sequence may be co-expressed with the nucleic acid sequence which encodes the AAVhu68 vp2 amino acid sequence of SEQ ID NO: 21 (about aa 138 to 736) without the vp1-unique region (about aa 1 to about 137), or a strand complementary thereto, the corresponding mRNA (nt 412 to 2211 of SEQ ID NO: 20), or a sequence at least 70% to at least 99% (e.g., at least 85%, at least 90%, at least 95%, at least 97%, at least 98% or at least 99%) identical to SEQ ID NO: 20 which encodes about aa 138 to 736 of SEQ ID NO: 21.

As described herein, a rAAVhu68 has a rAAVhu68 capsid produced in a production system expressing capsids from an AAVhu68 nucleic acid which encodes the vp1 amino acid sequence of SEQ ID NO: 21, and optionally additional nucleic acid sequences, e.g., encoding a vp3 protein free of the vp1 and/or vp2-unique regions. The rAAVhu68 resulting from production using a single nucleic acid sequence vp1 produces the heterogeneous populations of vp1 proteins, vp2 proteins and vp3 proteins. More particularly, the rAAVhu68 capsid contains subpopulations within the vp1 proteins, within the vp2 proteins and within the vp3 proteins which have modifications from the predicted amino acid residues in SEQ ID NO: 21. These subpopulations include, at a minimum, deamidated asparagine (N or Asn) residues. For example, asparagines in asparagine-glycine pairs are highly deamidated.

In one embodiment, the AAVhu68 vp1 nucleic acid sequence has the sequence of SEQ ID NO: 20, or a strand complementary thereto, e.g., the corresponding mRNA. In certain embodiments, the vp2 and/or vp3 proteins may be expressed additionally or alternatively from different nucleic acid sequences than the vp1, e.g., to alter the ratio of the vp proteins in a selected expression system. In certain embodiments, also provided is a nucleic acid sequence which encodes the AAVhu68 vp3 amino acid sequence of SEQ ID NO: 21 (about aa 203 to 736) without the vp1-unique region (about aa 1 to about aa 137) and/or vp2-unique regions (about aa 1 to about aa 202), or a strand complementary thereto, the corresponding mRNA (about nt 607 to about nt 2211 of SEQ ID NO: 20). In certain embodiments, also provided is a nucleic acid sequence which encodes the AAVhu68 vp2 amino acid sequence of SEQ ID NO: 21 (about aa 138 to 736) without the vp1-unique region (about aa 1 to about 137), or a strand complementary thereto, the corresponding mRNA (nt 412 to 2211 of SEQ ID NO: 20).

However, other nucleic acid sequences which encode the amino acid sequence of SEQ ID NO: 21 may be selected for use in producing rAAVhu68 capsids. In certain embodiments, the nucleic acid sequence has the nucleic acid sequence of SEQ ID NO: 20 or a sequence at least 70% to 99% identical, at least 75%, at least 80%, at least 85%, at least 90%, at least 95%, at least 97%, or at least 99% identical to SEQ ID NO: 20 which encodes SEQ ID NO: 21. In certain embodiments, the nucleic acid sequence has the nucleic acid sequence of SEQ ID NO: 20 or a sequence at least 70% to 99%, at least 75%, at least 80%, at least 85%, at least 90%, at least 95%, at least 97%, or at least 99% identical to about nt 412 to about nt 2211 of SEQ ID NO: 20 which encodes the vp2 capsid protein (about aa 138 to 736) of SEQ ID NO: 21. In certain embodiments, the nucleic acid sequence has the nucleic acid sequence of about nt 607 to about nt 2211 of SEQ ID NO: 20 or a sequence at least 70% to 99.%, at least 75%, at least 80%, at least 85%, at least 90%, at least 95%, at least 97%, or at least 99% identical to about nt 607 to about nt 2211 SEQ ID NO: 20 which encodes the vp3 capsid protein (about aa 203 to 736) of SEQ ID NO: 21.

It is within the skill in the art to design nucleic acid sequences encoding this rAAVhu68 capsid, including DNA (genomic or cDNA), or RNA (e.g., mRNA). In certain embodiments, the nucleic acid sequence encoding the AAVhu68 vp1 capsid protein is provided in SEQ ID NO: 20. In other embodiments, a nucleic acid sequence of 70% to 99.9% identity to SEQ ID NO: 20 may be selected to express the AAVhu68 capsid proteins. In certain other embodiments, the nucleic acid sequence is at least about 75% identical, at least 80% identical, at least 85%, at least 90%, at least 95%, at least 97% identical, or at least 99% to 99.9% identical to SEQ ID NO: 20. Such nucleic acid sequences may be codon-optimized for expression in a selected system (i.e., cell type) can be designed by various methods. This optimization may be performed using methods which are available on-line (e.g., GeneArt), published methods, or a company which provides codon optimizing services, e.g., DNA2.0 (Menlo Park, CA). One codon optimizing method is described, e.g., in US International Patent Publication No. WO 2015/012924, which is incorporated by reference herein in its entirety. See also, e.g., US Patent Publication No. 2014/0032186 and US Patent Publication No. 2006/0136184. Suitably, the entire length of the open reading frame (ORF) for the product is modified. However, in some embodiments, only a fragment of the ORF may be altered. By using one of these methods, one can apply the frequencies to any given polypeptide sequence and produce a nucleic acid fragment of a codon-optimized coding region which encodes the polypeptide. A number of options are available for performing the actual changes to the codons or for synthesizing the codon-optimized coding regions designed as described herein. Such modifications or synthesis can be performed using standard and routine molecular biological manipulations well known to those of ordinary skill in the art. In one approach, a series of complementary oligonucleotide pairs of 80-90 nucleotides each in length and spanning the length of the desired sequence are synthesized by standard methods. These oligonucleotide pairs are synthesized such that upon annealing, they form double stranded fragments of 80-90 base pairs, containing cohesive ends, e.g., each oligonucleotide in the pair is synthesized to extend 3, 4, 5, 6, 7, 8, 9, 10, or more bases beyond the region that is complementary to the other oligonucleotide in the pair. The single-stranded ends of each pair of oligonucleotides are designed to anneal with the single-stranded end of another pair of oligonucleotides. The oligonucleotide pairs are allowed to anneal, and approximately five to six of these double-stranded fragments are then allowed to anneal together via the cohesive single stranded ends, and then they ligated together and cloned into a standard bacterial cloning vector, for example, a TOPO® vector available from Invitrogen Corporation, Carlsbad, Calif. The construct is then sequenced by standard methods. Several of these constructs consisting of 5 to 6 fragments of 80 to 90 base pair fragments ligated together, i.e., fragments of about 500 base pairs, are prepared, such that the entire desired sequence is represented in a series of plasmid constructs. The inserts of these plasmids are then cut with appropriate restriction enzymes and ligated together to form the final construct. The final construct is then cloned into a standard bacterial cloning vector, and sequenced. Additional methods would be immediately apparent to the skilled artisan. In addition, gene synthesis is readily available commercially.

In certain embodiments, the asparagine (N) in N-G pairs in the rAAVhu68 vp1, vp2 and vp3 proteins are highly deamidated. In the case of the rAAVhu68 capsid protein, 4 residues (N57, N329, N452, N512) routinely display levels of deamidation >70% and it most cases >90% across various lots. Additional asparagine residues (N94, N253, N270, N304, N409, N477, and Q599) also display deamidation levels up to −20% across various lots. The deamidation levels were initially identified using a trypsin digest and verified with a chymotrypsin digestion.

In certain embodiments, an rAAVhu68 capsid contains subpopulations of AAV vp1, vp2 and/or vp3 capsid proteins having at least four asparagine (N) positions in the rAAVhu68 capsid proteins which are highly deamidated. In certain embodiments, about 20 to 50% of the N—N pairs (exclusive of N—N—N triplets) show deamidation. In certain embodiments, the first N is deamidated. In certain embodiments, the second N is deamidated. In certain embodiments, the deamidation is between about 15% to about 25% deamidation. Deamidation at the Q at position 259 of SEQ ID NO: 21 is about 8% to about 42% of the AAVhu68 vp1, vp2 and vp3 capsid proteins of an AAVhu68 protein.

In certain embodiments, the rAAVhu68 capsid is further characterized by an amidation in D297 the vp1, vp2 and vp3 proteins. In certain embodiments, about 70% to about 75% of the D at position 297 of the vp1, vp2 and/or vp3 proteins in a AAVhu68 capsid are amidated, based on the numbering of SEQ ID NO: 21. In certain embodiments, at least one Asp in the vp1, vp2 and/or vp3 of the capsid is isomerized to D-Asp. Such isomers are generally present in an amount of less than about 1% of the Asp at one or more of residue positions 97, 107, 384, based on the numbering of SEQ ID NO: 21.

In certain embodiments, a rAAVhu68 has an AAVhu68 capsid having vp1, vp2 and vp3 proteins having subpopulations comprising combinations of one, two, three, four or more deamidated residues at the positions set forth in the table below. Deamidation in the rAAV may be determined using 2D gel electrophoresis, and/or mass spectrometry, and/or protein modelling techniques. Online chromatography may be performed with an Acclaim PepMap column and a Thermo UltiMate 3000 RSLC system (Thermo Fisher Scientific) coupled to a Q Exactive HF with a NanoFlex source (Thermo Fisher Scientific). MS data is acquired using a data-dependent top-20 method for the Q Exactive HF, dynamically choosing the most abundant not-yet-sequenced precursor ions from the survey scans (200-2000 m/z). Sequencing is performed via higher energy collisional dissociation fragmentation with a target value of 1e5 ions determined with predictive automatic gain control and an isolation of precursors was performed with a window of 4 m/z. Survey scans were acquired at a resolution of 120,000 at m/z 200. Resolution for HCD spectra may be set to 30,000 at m/z200 with a maximum ion injection time of 50 ms and a normalized collision energy of 30. The S-lens RF level may be set at 50, to give optimal transmission of the m/z region occupied by the peptides from the digest. Precursor ions may be excluded with single, unassigned, or six and higher charge states from fragmentation selection. BioPharma Finder 1.0 software (Thermo Fischer Scientific) may be used for analysis of the data acquired. For peptide mapping, searches are performed using a single-entry protein FASTA database with carbamidomethylation set as a fixed modification; and oxidation, deamidation, and phosphorylation set as variable modifications, a 10-ppm mass accuracy, a high protease specificity, and a confidence level of 0.8 for MS/MS spectra. Examples of suitable proteases may include, e.g., trypsin or chymotrypsin. Mass spectrometric identification of deamidated peptides is relatively straightforward, as deamidation adds to the mass of intact molecule +0.984 Da (the mass difference between —OH and —NH₂ groups). The percent deamidation of a particular peptide is determined mass area of the deamidated peptide divided by the sum of the area of the deamidated and native peptides. Considering the number of possible deamidation sites, isobaric species which are deamidated at different sites may co-migrate in a single peak. Consequently, fragment ions originating from peptides with multiple potential deamidation sites can be used to locate or differentiate multiple sites of deamidation. In these cases, the relative intensities within the observed isotope patterns can be used to specifically determine the relative abundance of the different deamidated peptide isomers. This method assumes that the fragmentation efficiency for all isomeric species is the same and independent on the site of deamidation. It will be understood by one of skill in the art that a number of variations on these illustrative methods can be used. For example, suitable mass spectrometers may include, e.g., a quadrupole time of flight mass spectrometer (QTOF), such as a Waters Xevo or Agilent 6530 or an orbitrap instrument, such as the Orbitrap Fusion or Orbitrap Velos (Thermo Fisher). Suitably liquid chromatography systems include, e.g., Acquity UPLC system from Waters or Agilent systems (1100 or 1200 series). Suitable data analysis software may include, e.g., MassLynx (Waters), Pinpoint and Pepfinder (Thermo Fischer Scientific), Mascot (Matrix Science), Peaks DB (Bioinformatics Solutions). Still other techniques may be described, e.g., in X. Jin et al, Hu Gene Therapy Methods, Vol. 28, No. 5, pp. 255-267, published online Jun. 16, 2017.

| Deamidation Based on | | |
|---|---|---|
| Predicted AAVHu68 | Average % Based on VP1/VP2/VP3 | |
| (SEQ ID NO: 21) | Proteins in AAVhu68 Capsid | |
| Deamidated Residue + 1 | Broad Range of | |
| (Neighboring AA) | Percentages (%) | Narrow Ranges (%) |
| N57 | 78 to 100% | 80 to 100, 85 to 97 |
| (N-G) | | |
| N66 | 0 to 5 | 0, 1 to 5 |
| (N-E) | | |
| N94 | 0 to 15, | 0, 1 to 15, 5 to 12, 8 |
| (N-H) | | |
| N113 | 0 to 2 | 0, 1 to 2 |
| (N-L) | | |
| ~N253 | 10 to 25 | 15 to 22 |
| (N-N) | | |
| Q259 | 8 to 42 | 10 to 40, 20 to 35 |
| (Q-I) | | |
| ~N270 | 12 to 30 | 15 to 28 |
| (N-D) | | |
| ~N304 | 0 to 5 | 1 to 4 |
| (N-N) (position | | |
| 303 also N) | | |
| N319 | 0 to 5 | 0, 1 to 5, 1 to 3 |
| (N-I) | | |
| N329 * | 65 to 100 | 70 to 95, 85 to 95, 80 to |
| (N-G)*(position | | 100, 85 to 100, |
| 328 also N) | | |
| N336 | 0 to 100 | 0, 1 to 10, 25 to 100, 30 |
| (N-N) | | to 100, 30 to 95 |
| ~N409 | 15 to 30 | 20 to 25 |
| (N-N) | | |
| N452 | 75 to 100 | 80 to 100, 90 to 100, 95 |
| (N-G) | | to 100, |
| N477 | 0 to 8 | 0, 1 to 5 |
| (N-Y) | | |
| N512 | 65 to 100 | 70 to 95, 85 to 95, 80 to |
| (N-G) | | 100, 85 to 100, |
| ~N515 | 0 to 25 | 0, 1 to 10, 5 to 25, 15 to |
| (N-S) | | 25 |
| ~Q599 | 1 to 20 | 2 to 20, 5 to 15 |
| (Asn-Q-Gly) | | |
| N628 | 0 to 10 | 0, 1 to 10, 2 to 8 |
| (N-F) | | |
| N651 | 0 to 3 | 0, 1 to 3 |
| (N-T) | | |
| N663 | 0 to 5 | 0, 1 to 5, 2 to 4 |
| (N-K) | | |
| N709 | 0 to 25 | 0, 1 to 22, 15 to 25 |
| (N-N) | | |
| N735 | 0 to 40 | 0. 1 to 35, 5 to 50, 20 to |
| | | 35 |

In certain embodiments, the AAVhu68 capsid is characterized by having capsid proteins in which at least 45% of N residues are deamidated at least one of positions N57, N329, N452, and/or N512 based on the numbering of amino acid sequence of SEQ ID NO: 21. In certain embodiments, at least about 60%, at least about 70%, at least about 80%, or at least 90% of the N residues at one or more of these N-G positions (i.e., N57, N329, N452, and/or N512, based on the numbering of amino acid sequence of SEQ ID NO: 21) are deamidated. In these and other embodiments, an AAVhu68 capsid is further characterized by having a population of proteins in which about 1% to about 20% of the N residues have deamidations at one or more of positions: N94, N253, N270, N304, N409, N477, and/or Q599, based on the numbering of amino acid sequence of SEQ ID NO: 21. In certain embodiments, the AAVhu68 comprises at least a subpopulation of vp1, vp2 and/or vp3 proteins which are deamidated at one or more of positions N35, N57, N66, N94, N113, N252, N253, Q259, N270, N303, N304, N305, N319, N328, N329, N336, N409, N410, N452, N477, N515, N598, Q599, N628, N651, N663, N709, N735, based on the numbering of amino acid sequence of SEQ ID NO: 21, or combinations thereof. In certain embodiments, the capsid proteins may have one or more amidated amino acids.

Still other modifications are observed, most of which do not result in conversion of one amino acid to a different amino acid residue. Optionally, at least one Lys in the vp1, vp2 and vp3 of the capsid are acetylated. Optionally, at least one Asp in the vp1, vp2 and/or vp3 of the capsid is isomerized to D-Asp. Optionally, at least one S (Ser, Serine) in the vp1, vp2 and/or vp3 of the capsid is phosphorylated. Optionally, at least one T (Thr, Threonine) in the vp1, vp2 and/or vp3 of the capsid is phosphorylated. Optionally, at least one W (trp, tryptophan) in the vp1, vp2 and/or vp3 of the capsid is oxidized. Optionally, at least one M (Met, Methionine) in the vp1, vp2 and/or vp3 of the capsid is oxidized. In certain embodiments, the capsid proteins have one or more phosphorylations. For example, certain vp1 capsid proteins may be phosphorylated at position 149.

In certain embodiments, an rAAVhu68 capsid comprises a heterogeneous population of vp1 proteins which are the product of a nucleic acid sequence encoding the amino acid sequence of SEQ ID NO: 21, wherein the vp1 proteins comprise a Glutamic acid (Glu) at position 67 and/or a valine (Val) at position 157; a heterogeneous population of vp2 proteins optionally comprising a valine (Val) at position 157; and a heterogeneous population of vp3 proteins. The AAVhu68 capsid contains at least one subpopulation in which at least 65% of asparagines (N) in asparagine-glycine pairs located at position 57 of the vp1 proteins and at least 70% of asparagines (N) in asparagine-glycine pairs at positions 329, 452 and/or 512 of the vp1, v2 and vp3 proteins are deamidated, based on the residue numbering of the amino acid sequence of SEQ ID NO: 21, wherein the deamidation results in an amino acid change.

As discussed in more detail herein, the deamidated asparagines may be deamidated to aspartic acid, isoaspartic acid, an interconverting aspartic acid/isoaspartic acid pair, or combinations thereof. In certain embodiments, the rAAVhu68 are further characterized by one or more of: (a) each of the vp2 proteins is independently the product of a nucleic acid sequence encoding at least the vp2 protein of SEQ ID NO: 21; (b) each of the vp3 proteins is independently the product of a nucleic acid sequence encoding at least the vp3 protein of SEQ ID NO: 21; (c) the nucleic acid sequence encoding the vp1 proteins is SEQ ID NO: 21, or a sequence at least 70% to at least 99% (e.g., at least 85%, at least 90%, at least 95%, at least 97%, at least 98% or at least 99%) identical to SEQ ID NO: 20 which encodes the amino acid sequence of SEQ ID NO: 21. Optionally that sequence is used alone to express the vp1, vp2 and vp3 proteins. Alternatively, this sequence may be co-expressed with one or more of a nucleic acid sequence which encodes the AAVhu68 vp3 amino acid sequence of SEQ ID NO: 21 (about aa 203 to 736) without the vp1-unique region (about aa 1 to about aa 137) and/or vp2-unique regions (about aa 1 to about aa 202), or a strand complementary thereto, the corresponding mRNA (about nt 607 to about nt 2211 of SEQ ID NO: 20), or a sequence at least 70% to at least 99% (e.g., at least 85%, at least 90%, at least 95%, at least 97%, at least 98% or at least 99%) identical to SEQ ID NO: 20 which encodes aa 203 to 736 of SEQ ID NO: 21. Additionally, or alternatively, the vp1-encoding and/or the vp2-encoding sequence may be co-expressed with the nucleic acid sequence which encodes the AAVhu68 vp2 amino acid sequence of SEQ ID NO: 21 (about aa 138 to 736) without the vp1-unique region (about aa 1 to about 137), or a strand complementary thereto, the corresponding mRNA (nt 412 to 2211 of SEQ ID NO: 20), or a sequence at least 70% to at least 99% (e.g., at least 85%, at least 90%, at least 95%, at least 97%, at least 98% or at least 99%) identical to SEQ ID NO: 20 which encodes about aa 138 to 736 of SEQ ID NO: 21.

Additionally or alternatively, the rAAVhu68 capsid comprises at least a subpopulation of vp1, vp2 and/or vp3 proteins which are deamidated at one or more of positions N57, N66, N94, N113, N252, N253, Q259, N270, N303, N304, N305, N319, N328, N329, N336, N409, N410, N452, N477, N512, N515, N598, Q599, N628, N651, N663, N709, based on the numbering of SEQ ID NO: 21, or combinations thereof; (e) rAAVhu68 capsid comprises a subpopulation of vp1, vp2 and/or vp3 proteins which comprise 1% to 20% deamidation at one or more of positions N66, N94, N113, N252, N253, Q259, N270, N303, N304, N305, N319, N328, N336, N409, N410, N477, N515, N598, Q599, N628, N651, N663, N709, based on the numbering of SEQ ID NO: 21, or combinations thereof; (f) the rAAVhu68 capsid comprises a subpopulation of vp1 in which 65% to 100% of the N at position 57 of the vp1 proteins, based on the numbering of SEQ ID NO: 21, are deamidated; (g) the rAAVhu68 capsid comprises subpopulation of vp1 proteins in which 75% to 100% of the N at position 57 of the vp1 proteins are deamidated; (h) the rAAVhu68 capsid comprises subpopulation of vp1 proteins, vp2 proteins, and/or vp3 proteins in which 80% to 100% of the N at position 329, based on the numbering of SEQ ID NO: 21, are deamidated; (i) the rAAVhu68 capsid comprises subpopulation of vp1 proteins, vp2 proteins, and/or vp3 proteins in which 80% to 100% of the N at position 452, based on the numbering of SEQ ID NO: 21, are deamidated; (j) the rAAVhu68 capsid comprises subpopulation of vp1 proteins, vp2 proteins, and/or vp3 proteins in which 80% to 100% of the N at position 512, based on the numbering of SEQ ID NO: 21, are deamidated; (k) the rAAV comprises about 60 total capsid proteins in a ratio of about 1 vp1 to about 1 to 1.5 vp2 to 3 to 10 vp3 proteins; (1) the rAAV comprises about 60 total capsid proteins in a ratio of about 1 vp1 to about 1 vp2 to 3 to 9 vp3 proteins.

In certain embodiments, the AAVhu68 is modified to change the glycine in an asparagine-glycine pair, in order to reduce deamidation. In other embodiments, the asparagine is altered to a different amino acid, e.g., a glutamine which deamidates at a slower rate; or to an amino acid which lacks amide groups (e.g., glutamine and asparagine contain amide groups); and/or to an amino acid which lacks amine groups (e.g., lysine, arginine and histidine contain amide groups). As used herein, amino acids lacking amide or amine side groups refer to, e.g., glycine, alanine, valine, leucine, isoleucine, serine, threonine, cystine, phenylalanine, tyrosine, or tryptophan, and/or proline. Modifications such as described may be in one, two, or three of the asparagine-glycine pairs found in the encoded AAVhu68 amino acid sequence. In certain embodiments, such modifications are not made in all four of the asparagine-glycine pairs. Thus, a method is provided for reducing deamidation of rAAVhu68 and/or engineered rAAVhu68 variants having lower deamidation rates. Additionally, one or more other amide amino acids may be changed to a non-amide amino acid to reduce deamidation of the rAAVhu68.

These amino acid modifications may be made by conventional genetic engineering techniques. For example, a nucleic acid sequence containing modified AAVhu68 vp codons may be generated in which one to three of the codons encoding glycine at position 58, 330, 453 and/or 513 in SEQ ID NO: 21 (asparagine-glycine pairs) are modified to encode an amino acid other than glycine. In certain embodiments, a nucleic acid sequence containing modified asparagine codons may be engineered at one to three of the asparagine-glycine pairs located at position 57, 329, 452 and/or 512 in SEQ ID NO: 21, such that the modified codon encodes an amino acid other than asparagine. Each modified codon may encode a different amino acid. Alternatively, one or more of the altered codons may encode the same amino acid. In certain embodiments, these modified AAVhu68 nucleic acid sequences may be used to generate a mutant rAAVhu68 having a capsid with lower deamidation than the native hu68 capsid. Such mutant rAAVhu68 may have reduced immunogenicity and/or increase stability on storage, particularly storage in suspension form. As used herein, a “codon” refers to three nucleotides in a sequence which encodes an amino acid.

As used herein, “encoded amino acid sequence” refers to the amino acid which is predicted based on the translation of a known DNA codon of a referenced nucleic acid sequence being translated to an amino acid. The following table illustrates DNA codons and twenty common amino acids, showing both the single letter code (SLC) and three letter code (3LC).

| Amino Acid | SLC | 3 LC | DNA codons |
|---|---|---|---|
| Isoleucine | I | Ile | ATT, ATC, ATA |
| Leucine | L | Leu | CTT, CTC, CTA, CTG, TTA, TTG |
| Valine | V | Val | GTT, GTC, GTA, GTG |
| Phenylalanine | F | Phe | TTT, TTC |
| Methionine | M | Met | ATG |
| Cysteine | C | Cys | TGT, TGC |
| Alanine | A | Ala | GCT, GCC, GCA, GCG |
| Glycine | G | Gly | GGT, GGC, GGA, GGG |
| Proline | P | Pro | CCT, CCC, CCA, CCG |
| Threonine | T | Thr | ACT, ACC, ACA, ACG |
| Serine | S | Ser | TCT, TCC, TCA, TCG, AGT, AGC |
| Tyrosine | Y | Tyr | TAT, TAC |
| Tryptophan | W | Trp | TGG |
| Glutamine | Q | Gln | CAA, CAG |
| Asparagine | N | Asn | AAT, AAC |
| Histidine | H | His | CAT, CAC |
| Glutamic acid | E | Glu | GAA, GAG |
| Aspartic acid | D | Asp | GAT, GAC |
| Lysine | K | Lys | AAA, AAG |
| Arginine | R | Arg | CGT, CGC, CGA, CGG, AGA, AGG |
| Stop codons | Stop | | TAA, TAG, TGA |

As used herein, the term “clade” as it relates to groups of AAV refers to a group of AAV which are phylogenetically related to one another as determined using a Neighbor-Joining algorithm by a bootstrap value of at least 75% (of at least 1000 replicates) and a Poisson correction distance measurement of no more than 0.05, based on alignment of the AAV vp1 amino acid sequence. The Neighbor-Joining algorithm has been described in the literature. See, e.g., M. Nei and S. Kumar, Molecular Evolution and Phylogenetics (Oxford University Press, New York (2000). Computer programs are available that can be used to implement this algorithm. For example, the MEGA v2.1 program implements the modified Nei-Gojobori method. Using these techniques and computer programs, and the sequence of an AAV vp1 capsid protein, one of skill in the art can readily determine whether a selected AAV is contained in one of the clades identified herein, in another clade, or is outside these clades. See, e.g., G Gao, et al, J Virol, 2004 June; 78(10: 6381-6388, which identifies Clades A, B, C, D, E and F, GenBank Accession Numbers AY530553 to AY530629. See, also, WO 2005/033321.

Methods of generating the capsid, coding sequences therefore, and methods for production of rAAV viral vectors have been described. See, e.g., Gao, et al, Proc. Natl. Acad. Sci. U.S.A. 100 (10), 6081-6086 (2003) and US 2013/0045186A1.

The ITRs or other AAV components may be readily isolated or engineered using techniques available to those of skill in the art from an AAV. Such AAV may be isolated, engineered, or obtained from academic, commercial, or public sources (e.g., the American Type Culture Collection, Manassas, VA). Alternatively, the AAV sequences may be engineered through synthetic or other suitable means by reference to published sequences such as are available in the literature or in databases such as, e.g., GenBank, PubMed, or the like. AAV viruses may be engineered by conventional molecular biology techniques, making it possible to optimize these particles for cell specific delivery of nucleic acid sequences, for minimizing immunogenicity, for tuning stability and particle lifetime, for efficient degradation, for accurate delivery to the nucleus, etc.

In certain embodiments, the rAAV is a self-complementary AAV. “Self-complementary AAV” refers a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety.

In certain embodiments, the rAAV is nuclease-resistant. Such nuclease may be a single nuclease, or mixtures of nucleases, and may be endonucleases or exonucleases. A nuclease-resistant rAAV indicates that the AAV capsid has fully assembled and protects these packaged genomic sequences from degradation (digestion) during nuclease incubation steps designed to remove contaminating nucleic acids which may be present from the production process. In many instances, the rAAV described herein is DNase resistant.

The recombinant adeno-associated virus (AAV) described herein may be generated using techniques which are known. See, e.g., WO 2003/042397; WO 2005/033321, WO 2006/110689; U.S. Pat. No. 7,588,772 B2. Such a method involves culturing a host cell which contains a nucleic acid sequence encoding an AAV capsid; a functional rep gene; an expression cassette as described herein flanked by AAV inverted terminal repeats (ITRs); and sufficient helper functions to permit packaging of the expression cassette into the AAV capsid protein. Also provided herein is the host cell which contains a nucleic acid sequence encoding an AAV capsid; a functional rep gene; a vector genome as described; and sufficient helper functions to permit packaging of the vector genome into the AAV capsid protein. In one embodiment, the host cell is a HEK 293 cell. These methods are described in more detail in WO2017160360 A2, which is incorporated by reference herein.

Other methods of producing rAAV available to one of skill in the art may be utilized. Suitable methods may include without limitation, baculovirus expression system or production via yeast. See, e.g., Robert M. Kotin, Large-scale recombinant adeno-associated virus production. Hum Mol Genet. 2011 Apr. 15; 20(R1): R2-R6. Published online 2011 Apr. 29. doi: 10.1093/hmg/ddr141; Aucoin M G et al., Production of adeno-associated viral vectors in insect cells using triple infection: optimization of baculovirus concentration ratios. Biotechnol Bioeng. 2006 Dec. 20; 95(6):1081-92; SAMI S. THAKUR, Production of Recombinant Adeno-associated viral vectors in yeast. Thesis presented to the Graduate School of the University of Florida, 2012; Kondratov 0 et al. Direct Head-to-Head Evaluation of Recombinant Adeno-associated Viral Vectors Manufactured in Human versus Insect Cells, Mol Ther. 2017 Aug. 10. pii: S1525-0016(17)30362-3. doi: 10.1016/j.ymthe.2017.08.003. [Epub ahead of print]; Mietzsch M et al, OneBac 2.0: Sf9 Cell Lines for Production of AAV1, AAV2, and AAV8 Vectors with Minimal Encapsidation of Foreign DNA. Hum Gene Ther Methods. 2017 February; 28(1):15-22. doi: 10.1089/hgtb.2016.164.; Li L et al. Production and characterization of novel recombinant adeno-associated virus replicative-form genomes: a eukaryotic source of DNA for gene transfer. PLoS One. 2013 Aug. 1; 8(8):e69879. doi: 10.1371/journal.pone.0069879. Print 2013; Galibert L et al, Latest developments in the large-scale production of adeno-associated virus vectors in insect cells toward the treatment of neuromuscular diseases. J Invertebr Pathol. 2011 July; 107 Suppl:S80-93. doi: 10.1016/j.jip.2011.05.008; and Kotin R M, Large-scale recombinant adeno-associated virus production. Hum Mol Genet. 2011 Apr. 15; 20(R1):R2-6. doi: 10.1093/hmg/ddr141. Epub 2011 Apr. 29.

A variety of AAV purification methods are known in the art. See, e.g., WO 2017/160360 entitled “Scalable Purification Method for AAV9”, which is incorporated by reference herein, and describes methods generally useful for Clade F capsids. A two-step affinity chromatography purification followed by anion exchange resin chromatography are used to purify the vector drug product and to remove empty capsids. T The crude cell harvest may be subject steps such as concentration of the vector harvest, diafiltration of the vector harvest, microfluidization of the vector harvest, nuclease digestion of the vector harvest, filtration of microfluidized intermediate, crude purification by chromatography, crude purification by ultracentrifugation, buffer exchange by tangential flow filtration, and/or formulation and filtration to prepare bulk vector. An affinity chromatography purification followed anion exchange resin chromatography are used to purify the vector drug product and to remove empty capsids. In one example, for the Affinity Chromatography step, the diafiltered product may be applied to a Capture Select™ Poros-AAV2/9 affinity resin (Life Technologies) that efficiently captures the AAV2/9 serotype. Under these ionic conditions, a significant percentage of residual cellular DNA and proteins flow through the column, while AAV particles are efficiently captured. See, also, WO2021/158915; WO2019/241535; and WO 2021/165537.

Conventional methods for characterization or quantification of rAAV are available to one of skill in the art. To calculate empty and full particle content, VP3 band volumes for a selected sample (e.g., in examples herein an iodixanol gradient-purified preparation where # of GC=# of particles) are plotted against GC particles loaded. The resulting linear equation (y=mx+c) is used to calculate the number of particles in the band volumes of the test article peaks. The number of particles (pt) per 20 μL loaded is then multiplied by 50 to give particles (pt)/mL. Pt/mL divided by GC/mL gives the ratio of particles to genome copies (pt/GC). Pt/mL—GC/mL gives empty pt/mL. Empty pt/mL divided by pt/mL and ×100 gives the percentage of empty particles. Generally, methods for assaying for empty capsids and AAV vector particles with packaged genomes have been known in the art. See, e.g., Grimm et al., Gene Therapy (1999) 6:1322-1330; Sommer et al., Molec. Ther. (2003) 7:122-128. To test for denatured capsid, the methods include subjecting the treated AAV stock to SDS-polyacrylamide gel electrophoresis, consisting of any gel capable of separating the three capsid proteins, for example, a gradient gel containing 3-8% Tris-acetate in the buffer, then running the gel until sample material is separated, and blotting the gel onto nylon or nitrocellulose membranes, preferably nylon. Anti-AAV capsid antibodies are then used as the primary antibodies that bind to denatured capsid proteins, preferably an anti-AAV capsid monoclonal antibody, most preferably the B1 anti-AAV-2 monoclonal antibody (Wobus et al., J. Virol. (2000) 74:9281-9293). A secondary antibody is then used, one that binds to the primary antibody and contains a means for detecting binding with the primary antibody, more preferably an anti-IgG antibody containing a detection molecule covalently bound to it, most preferably a sheep anti-mouse IgG antibody covalently linked to horseradish peroxidase. A method for detecting binding is used to semi-quantitatively determine binding between the primary and secondary antibodies, preferably a detection method capable of detecting radioactive isotope emissions, electromagnetic radiation, or colorimetric changes, most preferably a chemiluminescence detection kit. For example, for SDS-PAGE, samples from column fractions can be taken and heated in SDS-PAGE loading buffer containing reducing agent (e.g., DTT), and capsid proteins were resolved on pre-cast gradient polyacrylamide gels (e.g., Novex). Silver staining may be performed using SilverXpress (Invitrogen, CA) according to the manufacturer's instructions or other suitable staining method, i.e. SYPRO ruby or coomassie stains. In one embodiment, the concentration of AAV vector genomes (vg) in column fractions can be measured by quantitative real time PCR (Q-PCR). Samples are diluted and digested with DNase I (or another suitable nuclease) to remove exogenous DNA. After inactivation of the nuclease, the samples are further diluted and amplified using primers and a TaqMan™ fluorogenic probe specific for the DNA sequence between the primers. The number of cycles required to reach a defined level of fluorescence (threshold cycle, Ct) is measured for each sample on an Applied Biosystems Prism 7700 Sequence Detection System. Plasmid DNA containing identical sequences to that contained in the AAV vector is employed to generate a standard curve in the Q-PCR reaction. The cycle threshold (Ct) values obtained from the samples are used to determine vector genome titer by normalizing it to the Ct value of the plasmid standard curve. End-point assays based on the digital PCR can also be used. As used herein, the terms genome copies (GC) and vector genomes (vg) in the context of a dose or dosage (e.g., GC/kg and vg/kg) are meant to be interchangeable.

In one aspect, an optimized q-PCR method is used which utilizes a broad spectrum serine protease, e.g., proteinase K (such as is commercially available from Qiagen). More particularly, the optimized qPCR genome titer assay is similar to a standard assay, except that after the DNase I digestion, samples are diluted with proteinase K buffer and treated with proteinase K followed by heat inactivation. Suitably samples are diluted with proteinase K buffer in an amount equal to the sample size. The proteinase K buffer may be concentrated to 2 fold or higher. Typically, proteinase K treatment is about 0.2 mg/mL, but may be varied from 0.1 mg/mL to about 1 mg/mL. The treatment step is generally conducted at about 55° C. for about 15 minutes, but may be performed at a lower temperature (e.g., about 37° C. to about 50° C.) over a longer time period (e.g., about 20 minutes to about 30 minutes), or a higher temperature (e.g., up to about 60° C.) for a shorter time period (e.g., about 5 to 10 minutes). Similarly, heat inactivation is generally at about 95° C. for about 15 minutes, but the temperature may be lowered (e.g., about 70 to about 90° C.) and the time extended (e.g., about 20 minutes to about 30 minutes). Samples are then diluted (e.g., 1000 fold) and subjected to TaqMan analysis as described in the standard assay.

Additionally, or alternatively, droplet digital PCR (ddPCR) may be used. For example, methods for determining single-stranded and self-complementary AAV vector genome titers by ddPCR have been described. See, e.g., M. Lock et al, Hu Gene Therapy Methods, Hum Gene Ther Methods. 2014 April; 25(2):115-25. doi: 10.1089/hgtb.2013.131. Epub 2014 Feb. 14.

Methods for determining the ratio among vp1, vp2 and vp3 of capsid protein are also available. See, e.g., Vamseedhar Rayaprolu et al, Comparative Analysis of Adeno-Associated Virus Capsid Stability and Dynamics, J Virol. 2013 December; 87(24): 13150-13160; Buller R M, Rose J A. 1978. Characterization of adenovirus-associated virus-induced polypeptides in KB cells. J. Virol. 25:331-338; and Rose J A, Maizel J V, Inman J K, Shatkin A J. 1971. Structural proteins of adenovirus-associated viruses. J. Virol. 8:766-770.

As used herein, the term “treatment” or “treating” refers to composition(s) and/or method(s) for the purposes of amelioration of one or more symptoms of Fabry disease, restore of a desired function of hGLA, or improvement of a biomarker of disease. In some embodiments, the term “treatment” or “treating” is defined encompassing administering to a subject one or more compositions described herein for the purposes indicated herein. “Treatment” can thus include one or more of reducing onset or progression of Fabry disease, preventing disease, reducing the severity of the disease symptoms, retarding their progression, removing the disease symptoms, delaying progression of disease, or increasing efficacy of therapy in a given subject.

It should be understood that the compositions in the rAAV described herein are intended to be applied to other compositions, regimens, aspects, embodiments and methods described across the Specification.

### 5. Pharmaceutical Compositions or Formulations

In certain embodiments, provided herein is a pharmaceutical composition comprising a vector, such as a rAAV, as described herein in a formulation buffer. In certain embodiments, the pharmaceutical composition is suitable for co-administering with a functional hGLA protein (ERT) (e.g. Fabrazyme) or chaperone therapy (e.g. Galafold (migalastat), Amicus Therapeutics). In one embodiment, provided is a pharmaceutical composition comprising a rAAV as described herein in a formulation buffer. In certain embodiments, the rAAV is formulated at about 1×10⁹ genome copies (GC)/mL to about 1×10¹⁴ GC/mL. In a further embodiment, the rAAV is formulated at about 3×10⁹ GC/mL to about 3×10¹³ GC/mL. In yet a further embodiment, the rAAV is formulated at about 1×10⁹ GC/mL to about 1×10¹³ GC/mL. In one embodiment, the rAAV is formulated at least about 1×10¹¹ GC/mL.

In certain embodiments, the pharmaceutical composition comprises the expression cassette comprising an hGLA coding sequence in a non-viral or viral vector system. This may include, e.g, naked DNA, naked RNA, an inorganic particle, a lipid or lipid-like particle, a chitosan-based formulation and others known in the art and described for example by Ramamoorth and Narvekar, as cited above). Such a non-viral vector system may include, e.g., a plasmid or non-viral genetic element, or a protein-based vector.

In certain embodiments, the pharmaceutical composition comprises a non-replicating viral vector. Suitable viral vectors may include any suitable delivery vector, such as, e.g., a recombinant adenovirus, a recombinant lentivirus, a recombinant bocavirus, a recombinant adeno-associated virus (AAV), or another recombinant parvovirus. In certain embodiments, the viral vector is a recombinant AAV for delivery of a hGLA to a patient in need thereof.

As used herein, a “stock” of rAAV refers to a population of rAAV. Despite heterogeneity in their capsid proteins due to deamidation, rAAV in a stock are expected to share an identical vector genome. A stock can include rAAV having capsids with, for example, heterogeneous deamidation patterns characteristic of the selected AAV capsid proteins and a selected production system. The stock may be produced from a single production system or pooled from multiple runs of the production system. A variety of production systems, including but not limited to those described herein, may be selected.

In one embodiment, the pharmaceutical composition comprises a vector that includes an expression cassette comprising an hGLA coding sequence, and a formulation buffer suitable for delivery via intracerebroventricular (ICV), intrathecal (IT), intracisternal or intravenous (IV) injection. In one embodiment, the expression cassette comprising the hGLA coding sequence is in packaged a recombinant AAV.

In one embodiment, the pharmaceutical composition comprises a functional hGLA polypeptide, or a functional fragment thereof, for delivery to a subject as an enzyme replacement therapy (ERT). Such pharmaceutical compositions are usually administered intravenously, however intradermal, intramuscular, or oral administration is also possible in some circumstances. The compositions can be administered for prophylactic treatment of individuals suffering from, or at risk of, Fabry disease. For therapeutic applications, the pharmaceutical compositions are administered to a patient suffering from established disease in an amount sufficient to reduce the concentration of accumulated metabolite and/or prevent or arrest further accumulation of metabolite. For individuals at risk of lysosomal enzyme deficiency disease, the pharmaceutical compositions are administered prophylactically in an amount sufficient to either prevent or inhibit accumulation of metabolite. The pharmaceutical compositions comprising an hGLA protein described herein are administered in a therapeutically effective amount. In general, a therapeutically effective amount can vary depending on the severity of the medical condition in the subject, as well as the subject's age, general condition, and gender. Dosages can be determined by the physician and can be adjusted as necessary to suit the effect of the observed treatment. In one aspect, provided herein is a pharmaceutical composition for ERT formulated to contain a unit dosage of a hGLA protein, or functional fragment thereof.

In certain embodiments, the formulation further comprises a surfactant, preservative, excipients, and/or buffer dissolved in the aqueous suspending liquid. In one embodiment, the buffer is PBS. In another embodiment, the buffer is an artificial cerebrospinal fluid (aCSF), e.g., Eliott's formulation buffer; or Harvard apparatus perfusion fluid (an artificial CSF with final Ion Concentrations (in mM): Na 150; K 3.0; Ca 1.4; Mg 0.8; P 1.0; Cl 155). Various suitable solutions are known including those which include one or more of: buffering saline, a surfactant, and a physiologically compatible salt or mixture of salts adjusted to an ionic strength equivalent to about 100 mM sodium chloride (NaCl) to about 250 mM sodium chloride, or a physiologically compatible salt adjusted to an equivalent ionic concentration.

Suitably, the formulation is adjusted to a physiologically acceptable pH, e.g., in the range of pH 6 to 8, or pH 6.5 to 7.5, pH 7.0 to 7.7, or pH 7.2 to 7.8. As the pH of the cerebrospinal fluid is about 7.28 to about 7.32, for intrathecal delivery, a pH within this range may be desired; whereas for intravenous delivery, a pH of 6.8 to about 7.2 may be desired. However, other pHs within the broadest ranges and these subranges may be selected for other route of delivery.

A suitable surfactant, or combination of surfactants, may be selected from among non-ionic surfactants that are nontoxic. In one embodiment, a difunctional block copolymer surfactant terminating in primary hydroxyl groups is selected, e.g., such as Pluronic® F68 [BASF], also known as Poloxamer 188, which has a neutral pH, has an average molecular weight of 8400. Other surfactants and other Poloxamers may be selected, i.e., nonionic triblock copolymers composed of a central hydrophobic chain of polyoxypropylene (poly (propylene oxide)) flanked by two hydrophilic chains of polyoxyethylene (poly (ethylene oxide)), SOLUTOL HS 15 (Macrogol-15 Hydroxystearate), LABRASOL (Polyoxy capryllic glyceride), polyoxy 10 oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits ×100 give the approximate molecular mass of the polyoxypropylene core, and the last digit ×10 gives the percentage polyoxyethylene content. In one embodiment Poloxamer 188 is selected. The surfactant may be present in an amount up to about 0.0005% to about 0.001% of the suspension.

In one example, the formulation may contain, e.g., buffered saline solution comprising one or more of sodium chloride, sodium bicarbonate, dextrose, magnesium sulfate (e.g., magnesium sulfate·7H2O), potassium chloride, calcium chloride (e.g., calcium chloride·2H2O), dibasic sodium phosphate, and mixtures thereof, in water. Suitably, for intrathecal delivery, the osmolarity is within a range compatible with cerebrospinal fluid (e.g., about 275 to about 290); see, e.g., emedicine.medscape.com/article/2093316-overview. Optionally, for intrathecal delivery, a commercially available diluent may be used as a suspending agent, or in combination with another suspending agent and other optional excipients. See, e.g., Elliotts B® solution [Lukare Medical].

In certain embodiments, the formulation may contain one or more permeation enhancers. Examples of suitable permeation enhancers may include, e.g., mannitol, sodium glycocholate, sodium taurocholate, sodium deoxycholate, sodium salicylate, sodium caprylate, sodium caprate, sodium lauryl sulfate, polyoxyethylene-9-laurel ether, or EDTA

In one embodiment, a frozen composition which contains an rAAV in a buffer solution as described herein, in frozen form, is provided. Optionally, one or more surfactants (e.g., Pluronic F68), stabilizers or preservatives is present in this composition. Suitably, for use, a composition is thawed and titrated to the desired dose with a suitable diluent, e.g., sterile saline or a buffered saline.

In certain embodiments, provided herein is a pharmaceutical composition comprising a vector, such as a rAAV, as described herein and a pharmaceutically acceptable carrier. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. Delivery vehicles such as liposomes, nanocapsules, microparticles, microspheres, lipid particles, vesicles, and the like, may be used for the introduction of the compositions of the present invention into suitable host cells. In particular, the rAAV vector may be formulated for delivery either encapsulated in a lipid particle, a liposome, a vesicle, a nanosphere, or a nanoparticle or the like. In one embodiment, a therapeutically effective amount of said vector is included in the pharmaceutical composition. The selection of the carrier is not a limitation of the present invention. Other conventional pharmaceutically acceptable carrier, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The phrase “pharmaceutically-acceptable” refers to molecular entities and compositions that do not produce an allergic or similar untoward reaction when administered to a host.

As used herein, the term “dosage” or “amount” can refer to the total dosage or amount delivered to the subject in the course of treatment, or the dosage or amount delivered in a single unit (or multiple unit or split dosage) administration.

Also, the replication-defective virus compositions can be formulated in dosage units to contain an amount of replication-defective virus that is in the range of about 1.0×10⁹ GC to about 1.0×10¹⁶ GC (to treat an average subject of 70 kg in body weight) including all integers or fractional amounts within the range, and preferably 1.0×10¹² GC to 1.0×10¹⁴ GC for a human patient. In one embodiment, the compositions are formulated to contain at least 1×10⁹, 2×10⁹, 3×10⁹, 4×10⁹, 5×10⁹, 6×10⁹, 7×10⁹, 8×10⁹, or 9×10⁹ GC per dose including all integers or fractional amounts within the range. In another embodiment, the compositions are formulated to contain at least 1×10¹⁰, 2×10¹⁰, 3×10¹⁰, 4×10¹⁰, 5×10¹⁰, 6×10¹⁰, 7×10¹⁰, 8×10¹⁰, or 9×10¹⁰ GC per dose including all integers or fractional amounts within the range. In another embodiment, the compositions are formulated to contain at least 1×10¹¹, 2×10¹¹, 3×10¹¹, 4×10¹¹, 5×10¹¹, 6×10¹¹, 7×10¹¹, 8×10¹¹, or 9×10¹¹ GC per dose including all integers or fractional amounts within the range. In another embodiment, the compositions are formulated to contain at least 1×10¹², 2×10¹², 3×10¹², 4×10¹², 5×10¹², 6×10¹², 7×10¹², 8×10¹², or 9×10¹² GC per dose including all integers or fractional amounts within the range. In another embodiment, the compositions are formulated to contain at least 1×10¹³, 2×10¹³, 3×10¹³, 4×10¹³, 5×10¹³, 6×10¹³, 7×10¹³, 8×10¹³, or 9×10¹³ GC per dose including all integers or fractional amounts within the range. In another embodiment, the compositions are formulated to contain at least 1×10¹⁴, 2×10¹⁴, 3×10¹⁴, 4×10¹⁴, 5×10¹⁴, 6×10¹⁴, 7×10¹⁴, 8×10¹⁴, or 9×10¹⁴ GC per dose including all integers or fractional amounts within the range. In another embodiment, the compositions are formulated to contain at least 1×10¹⁵, 2×10¹⁵, 3×10¹⁵, 4×10¹⁵, 5×10¹⁵, 6×10¹⁵, 7×10¹⁵, 8×10¹⁵, or 9×10¹⁵ GC per dose including all integers or fractional amounts within the range. In one embodiment, for human application the dose can range from 1×10¹⁰ to about 1×10¹² GC per dose including all integers or fractional amounts within the range.

In certain embodiments, provided is a pharmaceutical composition comprising a rAAV as described herein in a formulation buffer. In one embodiment, the rAAV is formulated at about 1×10⁹ genome copies (GC)/mL to about 1×10¹⁴ GC/mL. In a further embodiment, the rAAV is formulated at about 3×10⁹ GC/mL to about 3×10¹³ GC/mL. In yet a further embodiment, the rAAV is formulated at about 1×10⁹ GC/mL to about 1×10¹³ GC/mL. In one embodiment, the rAAV is formulated at least about 1×10¹¹ GC/mL. In one embodiment, the pharmaceutical composition comprising a rAAV as described herein is administrable at a dose of about 1×10⁹ GC per gram of brain mass to about 1×10¹⁴ GC per gram of brain mass.

In certain embodiments, the composition may be formulated in a suitable aqueous suspension media (e.g., a buffered saline) for delivery by any suitable route. The compositions provided herein are useful for systemic delivery of high doses of viral vector. For rAAV, a high dose may be at least 1×10¹³ GC or at least 1×10¹⁴ GC. However, for improved safety, the miRNA sequences provided herein may be included in expression cassettes and/or vector genomes which are delivered at other lower doses.

The aqueous suspension or pharmaceutical compositions described herein are designed for delivery to subjects in need thereof by any suitable route or a combination of different routes. In one embodiment, the pharmaceutical composition is formulated for delivery via intracerebroventricular (ICV), intrathecal (IT), or intracisternal injection. In one embodiment, the compositions described herein are designed for delivery to subjects in need thereof by intravenous (IV) injection. Alternatively, other routes of administration may be selected (e.g., oral, inhalation, intranasal, intratracheal, intraarterial, intraocular, intramuscular, and other parenteral routes). In certain embodiments, the composition is delivered by two different routes at essentially the same time.

As used herein, the terms “intrathecal delivery” or “intrathecal administration” refer to a route of administration for drugs via an injection into the spinal canal, more specifically into the subarachnoid space so that it reaches the cerebrospinal fluid (CSF). Intrathecal delivery may include lumbar puncture, intraventricular, suboccipital/intracisternal, and/or C1-2 puncture. For example, material may be introduced for diffusion throughout the subarachnoid space by means of lumbar puncture. In another example, injection may be into the cisterna magna. Intracisternal delivery may increase vector diffusion and/or reduce toxicity and inflammation caused by the administration. See, e.g., Christian Hinderer et al, Widespread gene transfer in the central nervous system of cynomolgus macaques following delivery of AAV9 into the cisterna magna, Mol Ther Methods Clin Dev. 2014; 1: 14051. Published online 2014 Dec. 10. doi: 10.1038/mtm.2014.51.

As used herein, the terms “intracisternal delivery” or “intracisternal administration” refer to a route of administration for drugs directly into the cerebrospinal fluid of the brain ventricles or within the cisterna magna cerebellomedularis, more specifically via a suboccipital puncture or by direct injection into the cisterna magna or via permanently positioned tube.

It should be understood that the compositions in the pharmaceutical compositions described herein are intended to be applied to other compositions, regimens, aspects, embodiments and methods described across the Specification.

### 6. Methods of Treatment

Provided herein are methods for Fabry disease comprising delivering a therapeutically effective amount of a nucleic acid sequence or expression cassette that includes a hGLA coding sequence, as provided herein. In particular, the methods include preventing, treating, and/or ameliorating symptoms of Fabry disease by delivering a therapeutically effective amount of a rAAV.hGLA or a composition that includes an hGLA polypeptide described herein to a patient in need thereof. In certain embodiments, a composition comprising an expression cassette as described herein is administrated to a subject in need thereof. In certain embodiments, the expression cassette is delivered via an rAAV.

As used herein, a “therapeutically effective amount” refers to the amount of a composition which delivers an amount of hGLA sufficient to ameliorate or treat one or more of the symptoms of Fabry disease. “Treatment” may include preventing the worsening of the symptoms of Fabry disease and possibly reversal of one or more of the symptoms thereof. A “therapeutically effective amount” for human patients may be predicted based on an animal model. See, C. Hinderer et al, Molecular Therapy (2014); 22 12, 2018-2027; A. Bradbury, et al, Human Gene Therapy Clinical Development. March 2015, 26(1): 27-37, which are incorporated herein by reference.

In certain embodiments, treatment includes preventing, treating, and/or ameliorating one or more symptoms of Fabry including, e.g., renal disease, cardiomyopathy, pain, fatigue, stroke, hearing loss, gastrointestinal disorders.

In certain embodiments, treatment includes delivering an expression cassette, nucleic acid, vector (e.g. rAAV), or polypeptide as described herein to one or more of the microvasculature, kidney cells, cardiac/heart cells, peripheral nerves, and cells of the central nervous system. In certain embodiments, treatment results in alpha-GalA substrate reduction in or more of cardiomyocytes, podocytes, vascular endothelial cells, and dorsal root ganglia. In certain embodiments, treatment results in alpha-GalA substrate reduction in the kidney. In certain embodiments, treatment results in alpha-GalA substrate reduction in the kidney tubules.

In certain embodiments, treatment includes replacing or supplementing a patient's defective alpha galactosidase A via rAAV-based gene therapy. As expressed from the rAAV vector described herein, expression levels of at least about 2% of normal levels as detected in the CSF, serum, neurons, or other tissue or fluid, may provide therapeutic effect. However, higher expression levels may be achieved. Such expression levels may be from 2% to about 100% of normal functional human GLA levels. In certain embodiments, higher than normal expression levels may be detected in serum or another biological fluid or tissue.

As used herein, the term “NAb titer” a measurement of how much neutralizing antibody (e.g., anti-AAV Nab) is produced, which neutralizes the physiologic effect of its targeted epitope (e.g., an AAV). Anti-AAV NAb titers may be measured as described in, e.g., Calcedo, R., et al., Worldwide Epidemiology of Neutralizing Antibodies to Adeno-Associated Viruses. Journal of Infectious Diseases, 2009. 199(3): p. 381-390, which is incorporated by reference herein.

In certain embodiments, the compositions provided herein are useful for delivery of a desired function hGLA product to patient, while repressing expression of the gene and/or gene product in dorsal root ganglion neurons. In certain embodiments, the method includes delivering a composition comprising an expression cassette comprising an hGLA coding sequence and miRNA target sequences to a patient. In certain embodiments, the method comprises delivering an expression cassette or vector genome that includes a miR-183 target sequence to repress transgene expression levels in the DRG. In certain embodiments, the method comprises delivering an expression cassette useful for repressing transgene expression in the DRG, wherein the expression cassette includes at least two miR183 target sequences, at least three miR183 target sequences, at least four miR183 target sequences, at least five miR183 target sequences, at least six miR183 target sequences, at least seven miR183 target sequences, or at least eight miR183 target sequences. In certain embodiments, the method comprises delivering an expression cassette useful for repressing transgene expression in the DRG, wherein the expression cassette includes at least two miR182 target sequences, at least three miR182 target sequences, at least four miR182 target sequences, at least five miR182 target sequences, at least six miR182 target sequences, at least seven miR182 target sequences, or at least eight miR182 target sequences. In certain embodiments, the expression cassettes include one or more miR182 target sequences and one or more miR183 target sequences.

Suitable volumes for delivery of the compositions provided and concentrations thereof may be determined by one of skill in the art. For example, volumes of about 1 μL to 150 mL may be selected, with the higher volumes being selected for adults. Typically, for newborn infants a suitable volume is about 0.5 mL to about 10 mL, for older infants, about 0.5 mL to about 15 mL may be selected. For toddlers, a volume of about 0.5 mL to about 20 mL may be selected. For children, volumes of up to about 30 mL may be selected. For pre-teens and teens, volumes up to about 50 mL may be selected. In still other embodiments, a patient may receive an intrathecal administration in a volume of about 5 mL to about 15 mL are selected, or about 7.5 mL to about 10 mL. Other suitable volumes and dosages may be determined. The dosage will be adjusted to balance the therapeutic benefit against any side effects and such dosages may vary depending upon the therapeutic application for which the recombinant vector is employed.

In certain embodiments, the composition comprising an rAAV as described herein is administrable at a dose of about 1×10⁹ GC per gram of brain mass to about 1×10¹⁴ GC per gram of brain mass. In certain embodiments, the rAAV is co-administered systemically at a dose of about 1×10⁹ GC per kg body weight to about 1×10¹³ GC per kg body weight.

In certain embodiments, the expression cassette is in a vector genome delivered in an amount of about 1×10⁹ GC per gram of brain mass to about 1×10¹³ genome copies (GC) per gram (g) of brain mass, including all integers or fractional amounts within the range and the endpoints. In another embodiment, the dosage is 1×10¹⁰ GC per gram of brain mass to about 1×10¹³ GC per gram of brain mass. In specific embodiments, the dose of the vector administered to a patient is at least about 1.0×10⁹ GC/g, about 1.5×10⁹ GC/g, about 2.0×10⁹ GC/g, about 2.5×10⁹ GC/g, about 3.0×10⁹ GC/g, about 3.5×10⁹ GC/g, about 4.0×10⁹ GC/g, about 4.5×10⁹ GC/g, about 5.0×10⁹ GC/g, about 5.5×10⁹ GC/g, about 6.0×10⁹ GC/g, about 6.5×10⁹ GC/g, about 7.0×10⁹ GC/g, about 7.5×10⁹ GC/g, about 8.0×10⁹ GC/g, about 8.5×10⁹ GC/g, about 9.0×10⁹ GC/g, about 9.5×10⁹ GC/g, about 1.0×10¹⁰ GC/g, about 1.5×10¹⁰ GC/g, about 2.0×10¹⁰ GC/g, about 2.5×10¹⁰ GC/g, about 3.0×10¹⁰ GC/g, about 3.5×10¹⁰ GC/g, about 4.0×10¹⁰ GC/g, about 4.5×10¹⁰ GC/g, about 5.0×10¹⁰ GC/g, about 5.5×10¹⁰ GC/g, about 6.0×10¹⁰ GC/g, about 6.5×10¹⁰ GC/g, about 7.0×10¹⁰ GC/g, about 7.5×10¹⁰ GC/g, about 8.0×10¹⁰ GC/g, about 8.5×10¹⁰ GC/g, about 9.0×10¹⁰ GC/g, about 9.5×10¹⁰ GC/g, about 1.0×10¹¹ GC/g, about 1.5×10¹¹ GC/g, about 2.0×10¹¹ GC/g, about 2.5×10¹¹ GC/g, about 3.0×10¹¹ GC/g, about 3.5×10¹¹ GC/g, about 4.0×10¹¹ GC/g, about 4.5×10¹¹ GC/g, about 5.0×10¹¹ GC/g, about 5.5×10¹¹ GC/g, about 6.0×10¹¹ GC/g, about 6.5×10¹¹ GC/g, about 7.0×10¹¹ GC/g, about 7.5×10¹¹ GC/g, about 8.0×10¹¹ GC/g, about 8.5×10¹¹ GC/g, about 9.0×10¹¹ GC/g, about 9.5×10¹¹ GC/g, about 1.0×10¹² GC/g, about 1.5×10¹² GC/g, about 2.0×10¹² GC/g, about 2.5×10¹² GC/g, about 3.0×10¹² GC/g, about 3.5×10¹² GC/g, about 4.0×10¹² GC/g, about 4.5×10¹² GC/g, about 5.0×10¹² GC/g, about 5.5×10¹² GC/g, about 6.0×10¹² GC/g, about 6.5×10¹² GC/g, about 7.0×10¹² GC/g, about 7.5×10¹² GC/g, about 8.0×10¹² GC/g, about 8.5×10¹² GC/g, about 9.0×10¹² GC/g, about 9.5×10¹² GC/g, about 1.0×10¹³ GC/g, about 1.5×10¹³ GC/g, about 2.0×10¹³ GC/g, about 2.5×10¹³ GC/g, about 3.0×10¹³ GC/g, about 3.5×10¹³ GC/g, about 4.0×10¹³ GC/g, about 4.5×10¹³ GC/g, about 5.0×10¹³ GC/g, about 5.5×10¹³ GC/g, about 6.0×10¹³ GC/g, about 6.5×10¹³ GC/g, about 7.0×10¹³ GC/g, about 7.5×10¹³ GC/g, about 8.0×10¹³ GC/g, about 8.5×10¹³ GC/g, about 9.0×10¹³ GC/g, about 9.5×10¹³ GC/g, or about 1.0×10¹⁴ GC/g brain mass.

In certain embodiments, the compositions provided herein are administered in combination an immunosuppressant. Currently, immunosuppressants for such co-therapy include, but are not limited to, a glucocorticoid, steroids, antimetabolites, T-cell inhibitors, a macrolide (e.g., a rapamycin or rapalog), and cytostatic agents including an alkylating agent, an anti-metabolite, a cytotoxic antibiotic, an antibody, or an agent active on immunophilin. The immune suppressant may include a nitrogen mustard, nitrosourea, platinum compound, methotrexate, azathioprine, mercaptopurine, fluorouracil, dactinomycin, an anthracycline, mitomycin C, bleomycin, mithramycin, IL-2 receptor- (CD25-) or CD3-directed antibodies, anti-IL-2 antibodies, ciclosporin, tacrolimus, sirolimus, IFN-β, IFN-γ, an opioid, or TNF-α (tumor necrosis factor-alpha) binding agent. In certain embodiments, the immunosuppressive therapy may be started 0, 1, 2, 7, or more days prior to the gene therapy administration. Such therapy may include co-administration of two or more drugs, the (e.g., prednisone, mycophenolate mofetil (MMF) and/or sirolimus (i.e., rapamycin)) on the same day. One or more of these drugs may be continued after gene therapy administration, at the same dose or an adjusted dose.

In certain embodiments, a rAAV as provided herein is administered in combination with a therapy (co-therapy), such as an enzyme-replacement therapy, chaperone therapy, substrate reduction therapy (e.g., Sanofi-Genzyme and Idorsia), and/or in combination with antihistamines or other medications which reduce the chance of infusion related reactions. In certain embodiments, the co-therapy is a functional hGLA protein (e.g. Fabrazyme® Sanofi-Genzyme; Replagal®; Shire; Protalix®, a plant based ERT) or a stabilized form of hGLA as provided herein or as described in PCT/US2019/05567, filed Oct. 10, 2019, which is incorporated herein by reference. Administration may be oral or by intravenous infusion to an outpatient and may be include dosages suitable for daily, every other day, weekly, every two weeks (e.g., 0.2 mg/kg body weight), monthly, or bimonthly administration. In certain, embodiments the co-therapy is a chaperone therapy (e.g. Galafold (migalastat, delivered orally in capsule form), Amicus Therapeutics). Appropriate therapeutically effective dosages of the co-therapies are selected by the treating clinician and include from about 1 μg/kg to about 500 mg/kg, from about 10 mg/kg to about 100 mg/kg, from about 20 mg/kg to about 100 mg/kg and approximately 20 mg/kg to approximately 50 mg/kg. In some embodiments, a suitable therapeutic dose is selected from, for example, 0.5, 0.75, 1, 5, 10, 15, 20, 30, 40, 50, 60, 70, 100, 150, 200, 250, 300, 400, or 500 mg/kg.

In certain embodiments, newborn babies (3 months old or younger) are treated in accordance with the methods described herein. In certain embodiments, babies that are 3 months old to 9 months old are treated in accordance with the methods described herein. In certain embodiments, children that are 9 months old to 36 months old are treated in accordance with the methods described herein. In certain embodiments, children that are 3 years old to 12 years old are treated in accordance with the methods described herein. In certain embodiments, children that are 12 years old to 18 years old are treated in accordance with the methods described herein. In certain embodiments, adults that are 18 years old or older are treated in accordance with the methods described herein.

In one embodiment, a patient with Fabry disease is a male or female of at least about 3 months to less than 12 months of age. In another embodiment, the patient with Fabry disease is a male or female and at least about 6 years to up to 18 years of age. In other embodiments, the subjects may be older or younger, and may be male or female.

It should be understood that the compositions in the methods described herein are intended to be applied to other compositions, regimens, aspects, embodiments and methods described across the Specification.

### 7. Kit

In certain embodiments, a kit is provided which includes a concentrated vector suspended in a formulation (optionally frozen), optional dilution buffer, and devices and components required for intravenous, intrathecal, intracerebroventricular, or intracisternal administration. In one embodiment, the kit provides sufficient buffer to allow for injection. Such buffer may allow for about a 1:1 to a 1:5 dilution of the concentrated vector, or more. Such a kit may include additional non-vector based active components where a combination therapy is utilized and/or anti-histamines, immunomodulators, or the like. In other embodiments, higher or lower amounts of buffer or sterile water are included to allow for dose titration and other adjustments by the treating clinician. In still other embodiments, one or more components of the device are included in the kit. Suitable dilution buffer is available, such as, a saline, a phosphate buffered saline (PBS) or a glycerol/PBS.

It should be understood that the compositions in kits described herein are intended to be applied to other compositions, regimens, aspects, embodiments and methods described across the Specification.

### 8. Device

In one aspect, the vectors provided herein may be administered intrathecally via the method and/or the device described, e.g., in WO 2017/136500, which is incorporated herein by reference in its entirety. Alternatively, other devices and methods may be selected. In summary, the method comprises the steps of advancing a spinal needle into the cisterna magna of a patient, connecting a length of flexible tubing to a proximal hub of the spinal needle and an output port of a valve to a proximal end of the flexible tubing, and after said advancing and connecting steps and after permitting the tubing to be self-primed with the patient's cerebrospinal fluid, connecting a first vessel containing an amount of isotonic solution to a flush inlet port of the valve and thereafter connecting a second vessel containing an amount of a pharmaceutical composition to a vector inlet port of the valve. After connecting the first and second vessels to the valve, a path for fluid flow is opened between the vector inlet port and the outlet port of the valve and the pharmaceutical composition is injected into the patient through the spinal needle, and after injecting the pharmaceutical composition, a path for fluid flow is opened through the flush inlet port and the outlet port of the valve and the isotonic solution is injected into the spinal needle to flush the pharmaceutical composition into the patient. This method and this device may each optionally be used for intrathecal delivery of the compositions provided herein. Alternatively, other methods and devices may be used for such intrathecal delivery.

It should be understood that the compositions in the devices described herein are intended to be applied to other compositions, regimens, aspects, embodiments and methods described across the Specification.

### EXAMPLES

The invention is now described with reference to the following examples. These examples are provided for the purpose of illustration only and the invention should in no way be construed as being limited to these examples but rather should be construed to encompass any and all variations that become evident as a result of the teachings provided herein.

### Example 1: A rAAVhu68.hGLA for Treatment of Fabry Disease

An engineered sequence that encodes for hGLA was cloned into an expression construct containing a CB7 promoter (a hybrid of a cytomegalovirus immediate-early enhancer and the chicken β-actin promoter), chicken β-actin intron (CI), WPRE, and a rabbit beta globin (rBG) polyadenylation sequence. The expression construct was flanked by AAV2 inverted terminal repeats and an AAVhu68 trans plasmid was used for encapsidation.

rAAVhu68.hGLA was produced by triple plasmid transfection of HEK293 cells with an AAV cis plasmid encoding the transgene cassette flanked by AAV ITRs, the AAV trans plasmid encoding the AAV2 rep and AAVhu68 cap genes (pAAV2/hu68.KanR), and the helper adenovirus plasmid (pAdAF6.KanR).

A. AAV Cis Plasmid

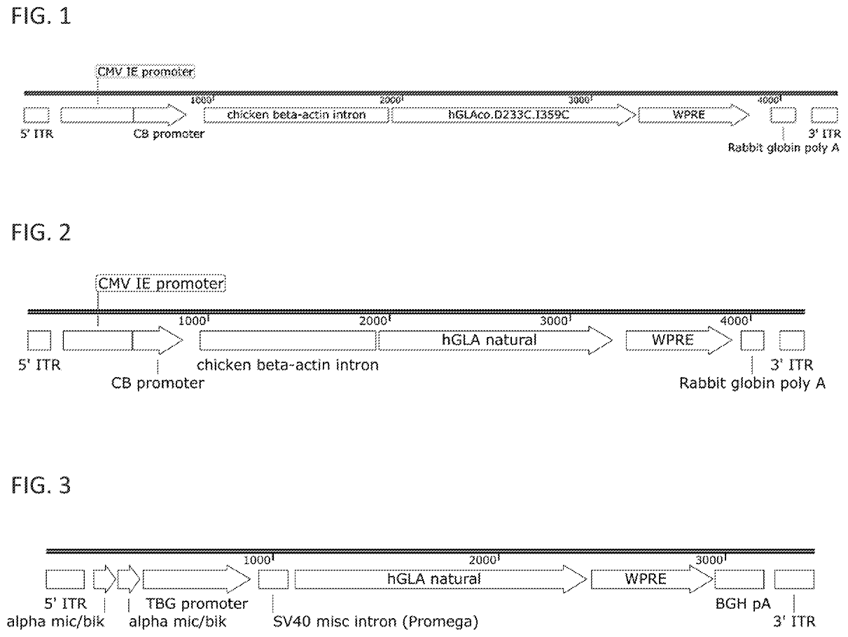

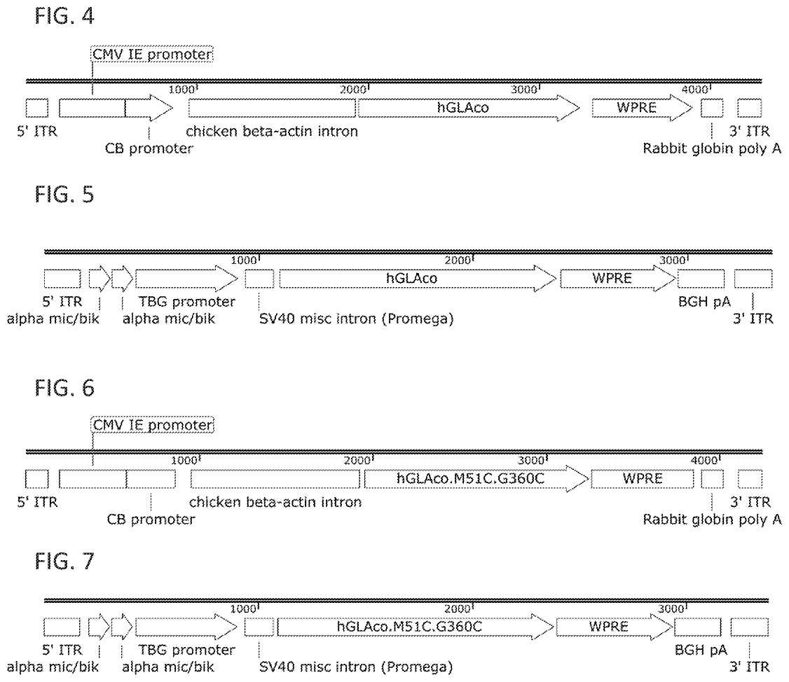

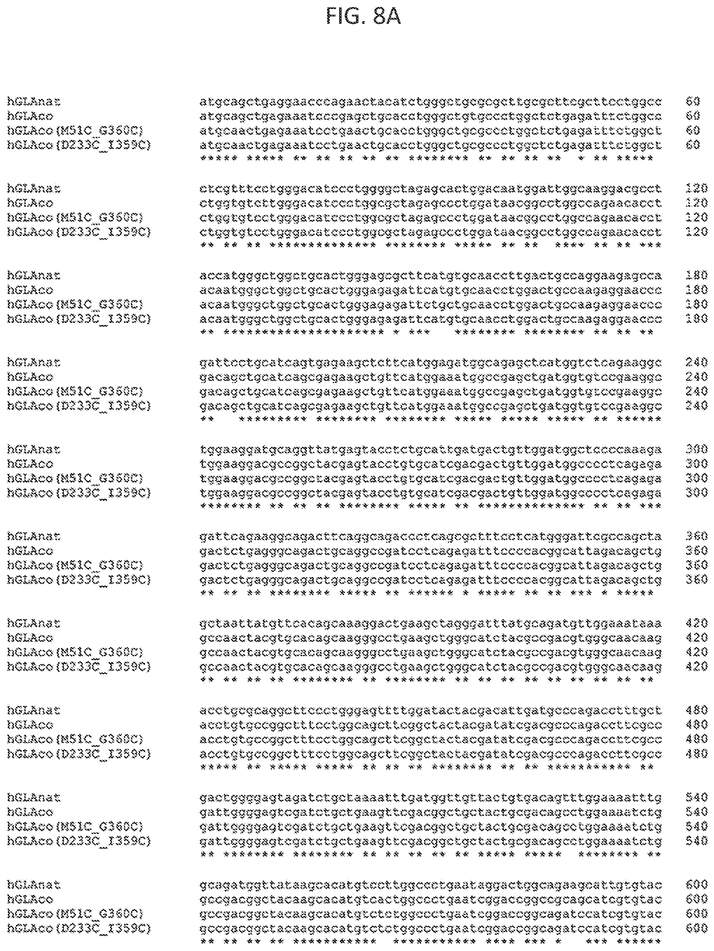

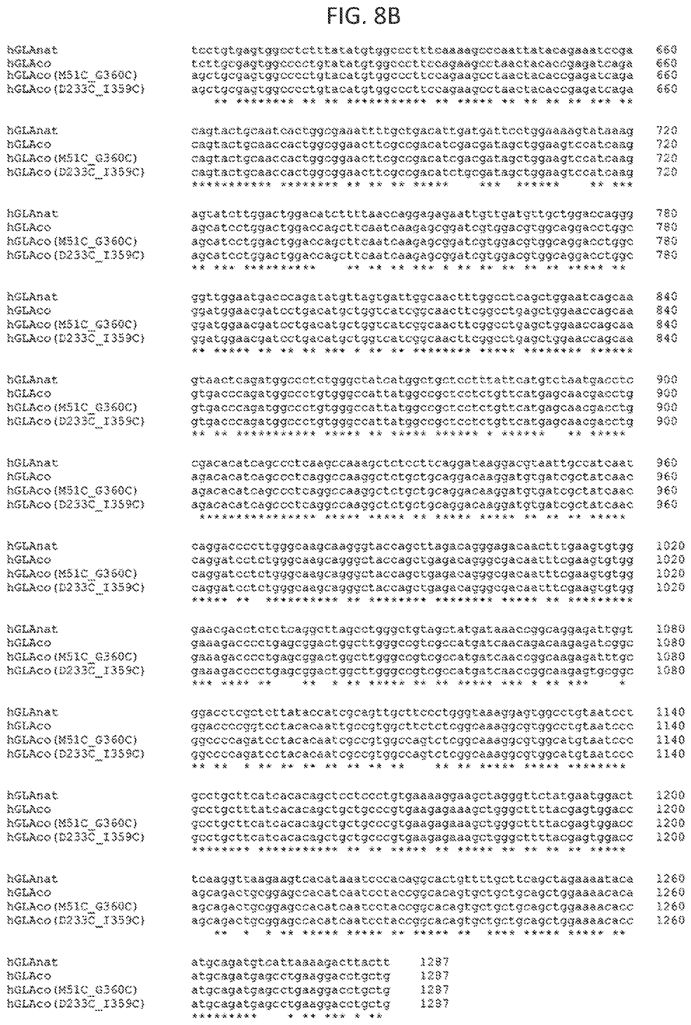

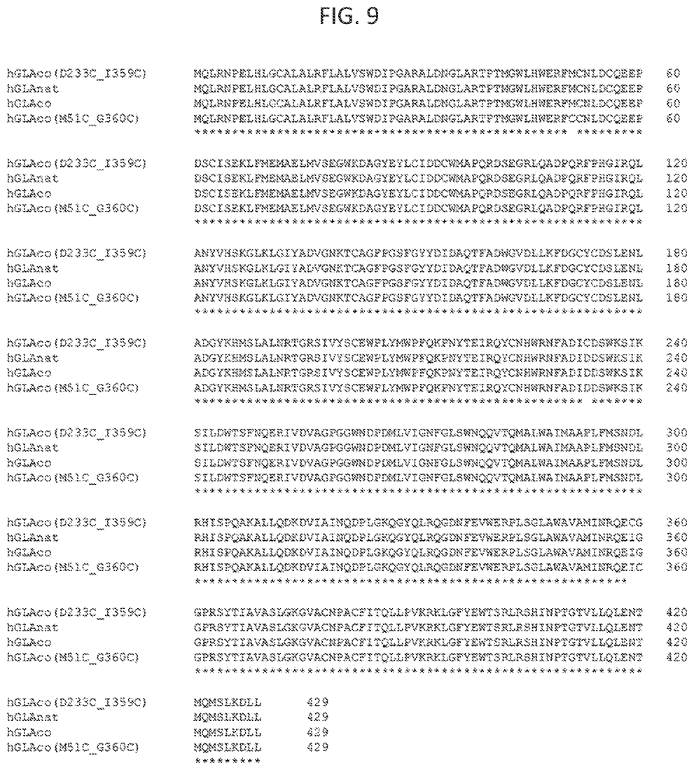

A map of the vector genome (SEQ ID NO: 6) is shown in FIG. 1. The vector genome contains the following sequence elements:

Inverted Terminal Repeats (ITRs): The ITRs are identical, reverse complementary sequences derived from AAV2 (130 bp, GenBank: NC 001401) that flank all components of the vector genome. The ITRs function as both the origin of vector DNA replication and the packaging signal for the vector genome when AAV and adenovirus helper functions are provided in trans. As such, the ITR sequences represent the only cis sequences required for vector genome replication and packaging.

CB7 promoter: This promoter is composed of a hybrid between a CMV IE enhancer and a chicken β-Actin promoter.

Human Cytomegalovirus Immediate-Early (CMV IE) Enhancer: This enhancer sequence obtained from human-derived CMV (GenBank: K03104.1) increases expression of downstream transgenes.

Chicken β-Actin (CB) Promoter: This ubiquitous promoter (GenBank: X00182.1) was selected to drive transgene expression in any cell type.

Chicken β-Actin Intron: The hybrid intron consists of a chicken β-actin splice donor (973 bp, GenBank: X00182.1) and rabbit β-globin splice acceptor element. The intron is transcribed, but removed from the mature mRNA by splicing, bringing together the sequences on either side of it. The presence of an intron in an expression cassette has been shown to facilitate the transport of mRNA from the nucleus to the cytoplasm, thus enhancing the accumulation of the steady level of mRNA for translation. This is a common feature in gene vectors intended for increased levels of gene expression.

Coding sequence: An engineered cDNA (SEQ ID NO: 4) that encodes hGLA (SEQ ID NO: 7) having cysteine residues at positions 233 and 359 (D233C.I359C) (431 amino acids).

Woodchuck hepatitis virus post-transcriptional regulatory element (WPRE): A cis-acting RNA element derived from the Woodchuck Hepatitis Virus (WHV) has been inserted in the 3′ untranslated region of the coding sequence upstream of the polyA signal. The WPRE is a hepadnavirus-derived sequence, and has been previously used as a cis-acting regulatory module in viral gene vectors to achieve sufficient levels of transgene product expression and to improve the viral titers during manufacturing. The WPRE is believed to increase transgene product expression by improving transcript termination and enhancing 3′ end transcript processing, thereby increasing the amount of polyadenylated transcripts and the size of the polyA tail, and resulting in more transgene mRNA available for translation. The WPRE included in the cis plasmid is a mutated version containing five point mutations in the putative promoter region of the woodchuck hepatitis virus X protein (WHX) protein open reading frame (ORF), along with an additional point mutation in the start codon of the WHX protein ORF (ATG mutated to TTG). This mutant WPRE (termed mut6) is considered sufficient to eliminate expression of truncated WHX protein based on sensitive flow cytometry analyses of various human cell lines transduced with lentivirus containing a WPRE mut6-GFP fusion construct (Zanta-Boussif et al., 2009)

The WPRE is a hepadnavirus-derived sequence, and has been previously used as a cis-acting regulatory module in viral gene vectors to achieve sufficient levels of transgene product expression and to improve the viral titers during manufacturing.

Rabbit β-Globin Polyadenylation Signal (rBG PolyA): The rBG PolyA signal (127 bp, GenBank: V00882.1) facilitates efficient polyadenylation of the transgene mRNA in cis. This element functions as a signal for transcriptional termination, a specific cleavage event at the 3′ end of the nascent transcript and the addition of a long polyadenyl tail.

B. Trans Plasmid: pAAV2/1.KanR (p0068)

The AAV2/hu68 trans plasmid is pAAV2/hu68.KanR (p0068). The pAAV2/hu68.KanR plasmid is 8030 bp in length and encodes four wild type AAV2 replicase (Rep) proteins required for the replication and packaging of the AAV vector genome. The pAAV2/hu68.KanR plasmid also encodes three WT AAVhu68 virion protein capsid (Cap) proteins, which assemble into a virion shell of the AAV serotype hu68 to house the AAV vector genome.

C. Adenovirus Helper Plasmid: pAdDeltaF6(KanR)

The adenovirus helper plasmid pAdDeltaF6(KanR) is 15,770 bp in size. The plasmid contains the regions of adenovirus genome that are important for AAV replication; namely, E2A, E4, and VA RNA (the adenovirus E1 functions are provided by the HEK293 cells). However, the plasmid does not contain other adenovirus replication or structural genes. The plasmid does not contain the cis elements critical for replication, such as the adenoviral ITRs; therefore, no infectious adenovirus is expected to be generated. The plasmid was derived from an E1, E3-deleted molecular clone of Ad5 (pBHG10, a pBR322-based plasmid). Deletions were introduced into Ad5 to eliminate expression of unnecessary adenovirus genes and reduce the amount of adenovirus DNA from 32 kb to 12 kb. Finally, the ampicillin resistance gene was replaced by the kanamycin resistance gene to create pAdeltaF6(KanR). The E2, E4, and VAI adenoviral genes that remain in this plasmid, along with E1, which is present in HEK293 cells, are necessary for AAV vector production.

### Example 2: Methods

### Transgene Product Expression—Cellular Distribution

To characterize the distribution of transduction and transgene product expression after IV administration of AAVhu69.hGLA vectors and correlate it with any observed histological improvements in the disease phenotype, both mRNA and protein localization were evaluated. The kidney, DRG, and heart tissues were selected for evaluation because they are disease-relevant target tissues for treating Fabry disease. Human GLA mRNA was evaluated by in situ hybridization (ISH) in mice and NHPs. Human GLA protein was evaluated by immunohistochemistry (IHC) or immunofluorescence (IF) in mice and NHPs. The sampling time points in mice and NHPs were selected to capture expression during the expected stable plateau of transgene product expression.

### Transgene Product Expression—Functional Activity

To evaluate whether the transgene product observed by ISH and IHC was functional in mice and NHPs, a GLA enzyme activity assay was performed. The kidney, heart, liver, and DRG tissues were selected because they are disease-relevant target tissues for treating Fabry disease (kidney, heart) and/or are readily transduced after IV gene therapy (liver, heart, DRG). Transgene product expression was not evaluated in DRG of mice due to their small size, while all other tissues were assessed in mice and NHPs. The sampling time points in mice and NHPs were selected to capture the stable plateau transgene expression. The GLA activity assay could not distinguish between the human GLA transgene product and endogenous mouse or NHP GLA, and therefore some background activity could be expected in untreated animals at baseline.

### Thermosensory Function

The hotplate assay was performed because it measures thermosensory deficits in mice, which are believed to be similar to the touch, pain, and thermal sensation deficits described in Fabry patients secondary to DRG neuron lysosomal storage and dysfunction. A decrease in latency response would indicate an improvement in the Fabry disease phenotype.

### Kidney Function

BUN, urine osmolality, and urine volume were evaluated because they are biomarkers for kidney function. A decrease in BUN levels would indicate an improvement in the Fabry disease phenotype. An increase in urine osmolarity would indicate increased ability to concentrate urine due to increased kidney function, which represents an improvement in the Fabry disease phenotype. A decrease in urine volume would indicate an improvement in the Fabry disease phenotype.

### Lysosomal Storage (GL-3 Immunohistochemistry on Tissues)

The GLA enzyme deficiency results in accumulation of the enzyme's toxic substrate, GL-3. The IHC for GL-3 was therefore performed on the DRG and kidneys because these are organs that reproducibly show marked storage in both the classic (Gla KO) and aggravated (Gla KO/TgG3S) Fabry mouse models and that are target organs of pathology in Fabry disease patients (causing neuropathic pain and fatal kidney failure, respectively). GL-3 IHC was performed on the heart because aggravated (Gla KO/TgG3S) Fabry mice also exhibit storage in this organ. Reduced GL-3 storage would indicate an improvement in the Fabry disease phenotype. GL-3 IHC sections were also stained with hematoxylin and eosin (H&E) to better visualize tissue morphology and detect potential adverse treatment-related findings. Lysosomal Storage (GL-3 and Lyso-Gb3 Quantification by LC-MS/MS)

Storage of GL-3 was quantified in tissues and lyso-Gb3 was quantified in plasma or serum by liquid chromatography with tandem mass spectrometry (LC-MS/MS). Storage was quantified in target organs because GL-3 is the main substrate of the GLA enzyme and there is direct evidence for a relationship between the severity of substrate accumulation and the severity of Fabry disease. A decrease in GL-3 storage would indicate an improvement in the Fabry disease phenotype.

### Example 3: Natural History Study of the Aggravated (Gla KO/TgG3S) and Classic (Gla KO) Mouse Models of Fabry Disease

A natural history study was performed to characterize the disease progression of the Fabry aggravated mouse model (Gla KO/TgG3S) and define the best pharmacology endpoints and therapeutic window for efficacy studies.

At birth, 41 mice were enrolled in the study, including Gla WT males or Gla′ heterozygote females with no TgG3S allele (Control; 5 males and 6 females), Gla KO with no TgG3S allele (Gla^{-/-}; 5 males and 5 females), Gla WT males or Gla heterozygote females with one allele of TgG3S (TgG3S⁺; 5 males and 5 females), and Gla KO with one allele of TgG3S (Gla KO/TgG3S; 5 males and 5 females). Body weights, hotplate performance, serum BUN levels, and urine osmolality were assessed at regular intervals. Necropsies were performed at 36 weeks of age, which is close to the published humane endpoint for this model. Brain, spinal cord, DRG, heart, kidney, liver, skin, small intestine, and large intestine were collected at necropsy for histology and evaluation of GL-3 storage (GL-3 IHC and quantification by LC-MS/MS).

Study Design: Natural History Study in Aggravated Fabry Gla^{-/-}/TgG3S⁺ Mouse Model

| | Age | | | | | | |
|---|---|---|---|---|---|---|---|
| | Weeks: | | | | | | |
| | 0 | 6 | 12 | 18 | 25 | 30 | 36 |
| | Weeks | Weeks | Weeks | Weeks | Weeks | Weeks | Weeks |
| | Months: | | | | | | |
| | 0 | 1.5 | 3 | 4.5 | 6.25 | 7.5 | 9 |
| | Months | Months | Months | Months | Months | Months | Months |
| Viability checks | X | | | | | | |
| (survival monitoring) | | | | | | | |
| Body weights | | X | X | X | X | X | X |
| Hot plate performance | | X | X | X | X | X | X |
| BUN levels (serum) | | X | X | X | X | X | X |
| Urine osmolarity | | X | X | X | X | X | X |
| Necropsy and sample | | | | | | | X |
| collection^{a} | | | | | | | |

^{a}Tissues were collected for evaluation of histopathology.
Abbreviations: BUN, blood urine nitrogen; Gla, alpha galactosidase A; TgG3S, human Gb3 synthase-transgenic.

All mice survived to the scheduled necropsy at 36 weeks except for two Gla KO/TgG3S mice euthanized due to disease-related body weight loss at 33 weeks and 35 weeks.

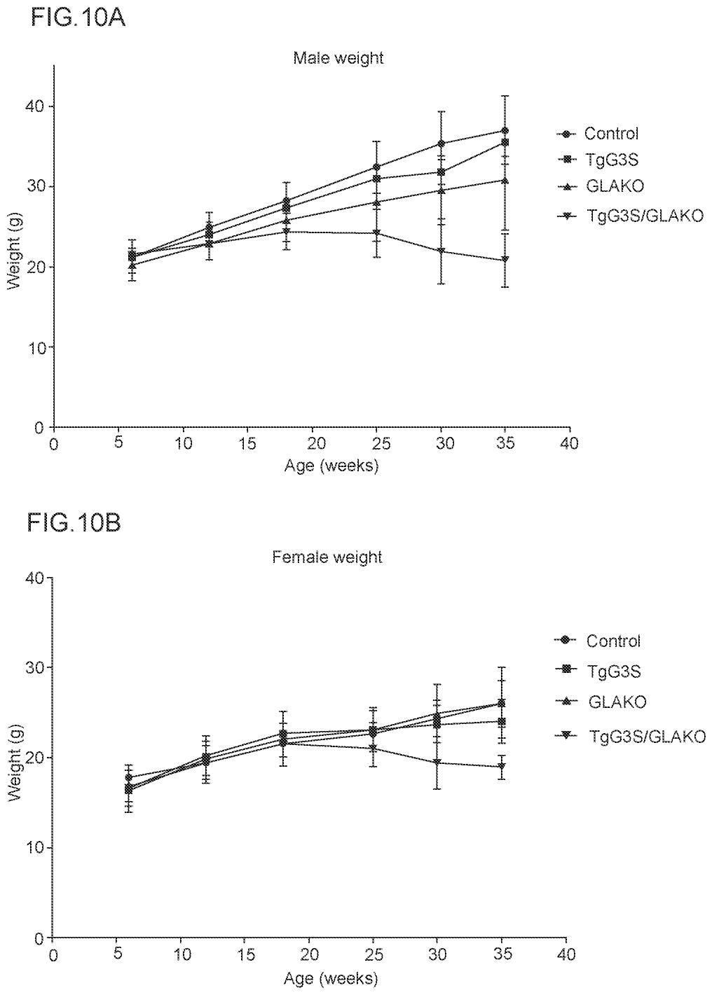

Control, GlaKO, and TgG3S mice gained weight at every time point throughout the 10 study (FIG. 10A and FIG. 10B). In contrast, the body weight of GlaKO/TgG3S mice peaked at 18 weeks (24.4 g for males and 21.5 g for females), after which the mice began to lose weight until necropsy.

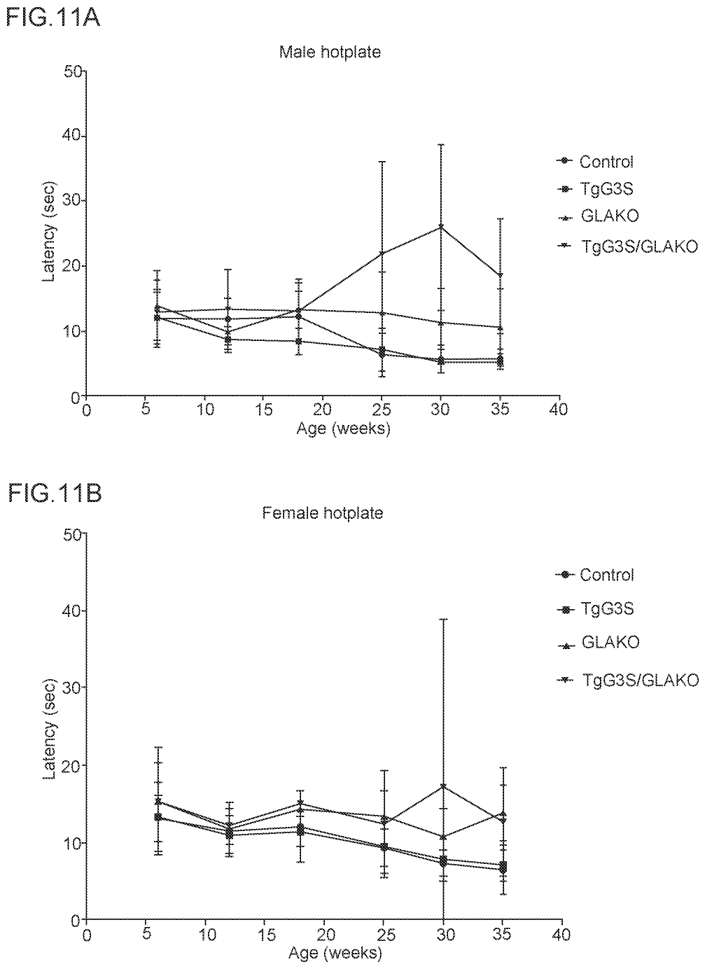

Male and female TgG3S mice exhibited a similar hotplate latency as sex-matched control animals throughout the study, indicating a normal sensory response. Male and female GlaKO mice exhibited a slightly longer average hotplate latency compared to the sex-matched controls from 25 weeks of age to the end of the study, indicating a slightly reduced sensory response. In contrast, male GlaKO/TgG3S mice demonstrated a substantially longer average latency response than both the control or GlaKO mice (male or female) from 25 weeks of age to the end of the study, indicating that male GlaKO/TgG3S mice have a more severe sensory deficit than both male and female GlaKO mice. Female GlaKO/TgG3S mice also exhibited a slightly longer hotplate latency response than control mice, but the latencies were similar to the female GlaKO mice, suggesting that sensory deficit for the two female Fabry mouse models are similar (FIG. 11A and FIG. 11B).

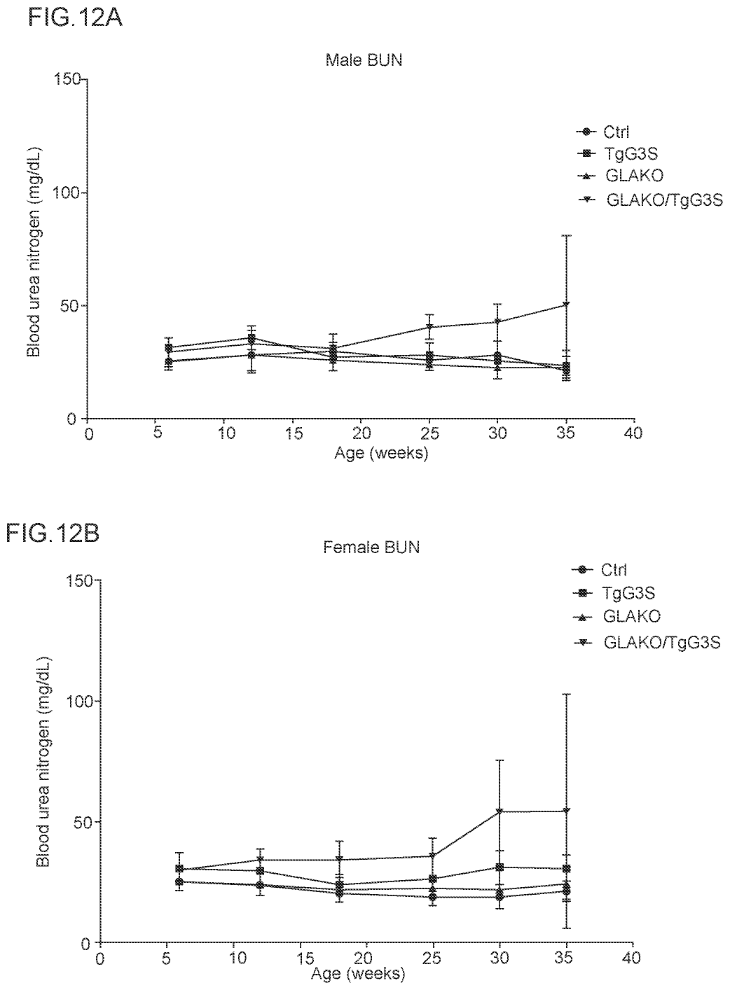

Male and female TgG3S and Gla KO mice displayed similar serum BUN levels as their sex-matched controls throughout the study, which was indicative of normal kidney function. In contrast, both male and female Gla KO/TgG3 S mice exhibited elevated BUN levels by 25 weeks of age when compared to sex-matched controls. BUN levels generally increasing over the course of the study for both males and female, suggesting decreasing kidney function. BUN levels were generally similar for male and female Gla KO/TgG3 S mice throughout the study (FIG. 12A and FIG. 12B).

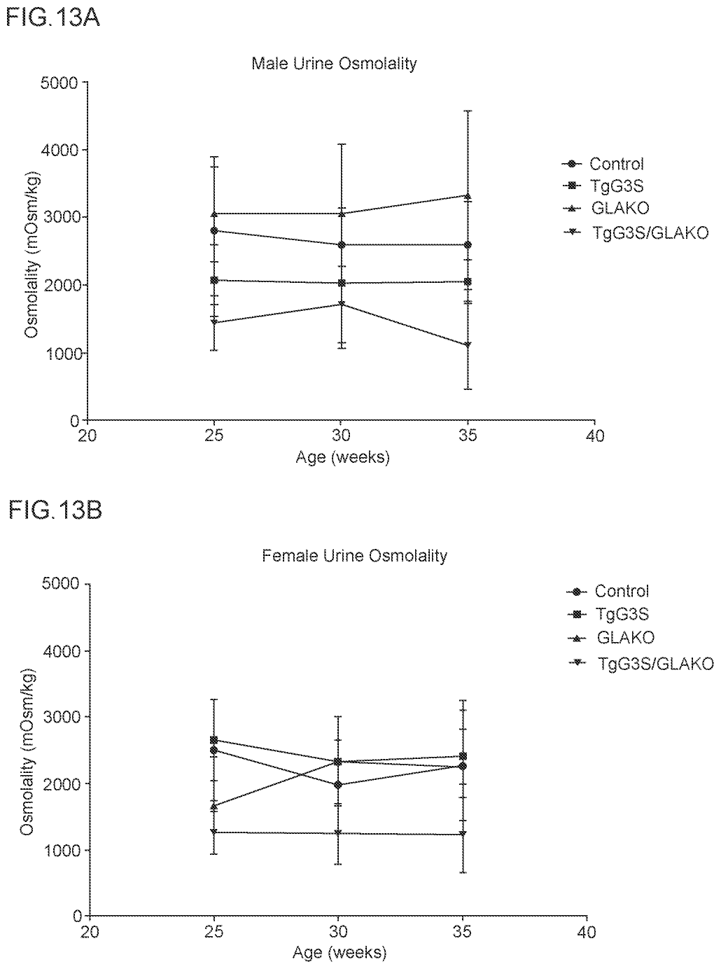

Intra-animal variability for urine osmolality was observed in the study. However, male and female Gla KO/TgG3S mice generally exhibited lower average urine osmolality than sex-matched controls and Gla KO mice from 25 weeks of age until the end of the study, suggesting failure to concentrate urine due to reduced kidney function (FIG. 13A and FIG. 13B).

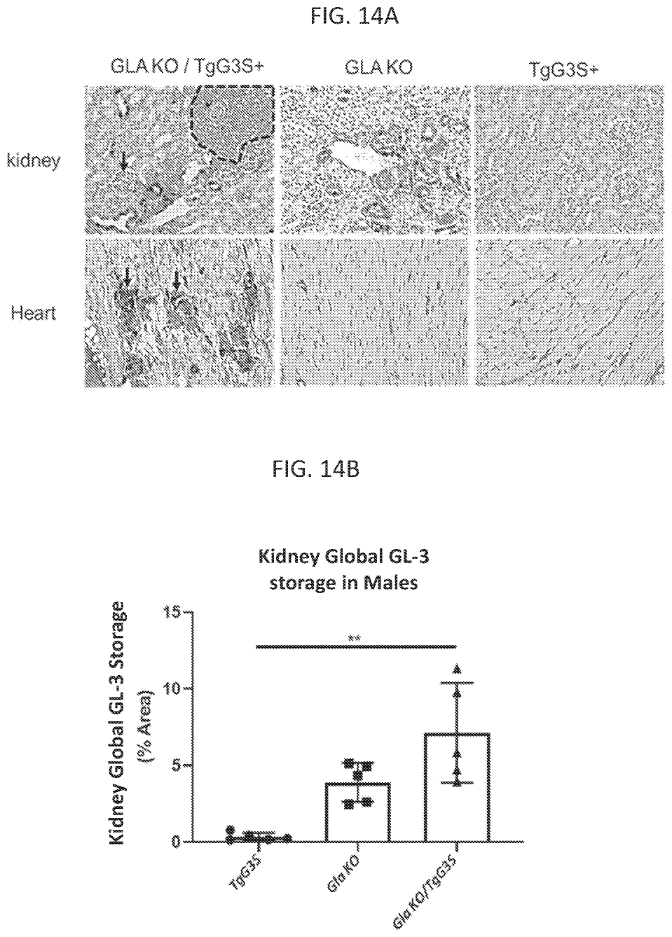

More pronounced GL-3 storage in the kidneys along with secondary lesions of nephritis and tubular necrosis was observed in male Gla KO/TgG3S mice compared to Gla KO or WT/TgG3S mice when evaluated by IHC (FIG. 14A). The extent of GL-3 storage and secondary pathology was greater in Gla KO/TgG3S mice compared to Gla KO mice. In the kidneys of Gla KO/TgG3S mice, storage material was seen in both tubules and glomeruli cells, whereas GL-3 storage was only seen in the tubules of Gla KO mice. In addition to greater storage in tubules and glomeruli, some Gla KO/TgG3S mice presented secondary inflammatory and degenerative lesions in the kidney (tubular degeneration, necrosis, and secondary interstitial mononuclear nephritis) that was not seen in any Gla KO mice. The heart, which did not exhibit any GL-3 storage or secondary lesions in Gla KO mice, exhibited some GL-3 storage material in cardiomyocytes as well as cardiomyocyte necrosis and mineralization in some Gla KO/TgG3S animals.

Quantification of GL-3 IHC confirmed that male Gla KO/TgG3S mice had significantly higher levels of GL-3 storage throughout the kidney than that of Gla KO or WT/TgG3S mice, with WT/TgG3S mice having the lowest levels of GL-3 storage among the 3 mouse models (FIG. 14B).

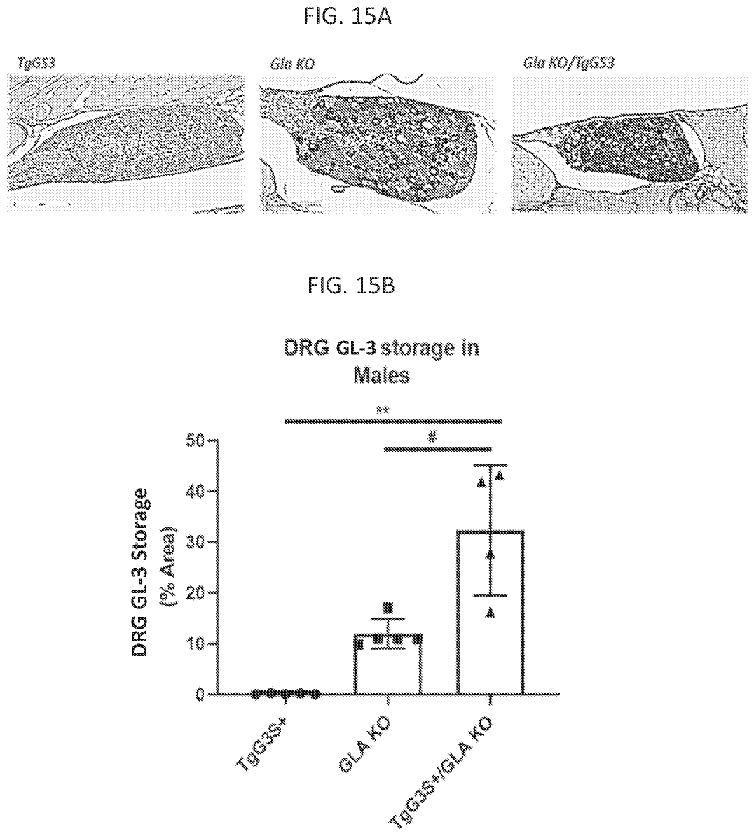

Male Gla KO/TgG3S mice exhibited substantial GL-3 storage in DRG sensory neurons when evaluated by IHC (FIG. 15A). While male Gla KO mice also displayed GL-3 storage in DRG sensory neurons, very little DRG GL-3 storage was observed in male WT/TgGS3 mice. Quantification of IHC staining revealed that DRG GL-3 storage was significantly increased in Gla KO/TgG3S mice compared to the Gla KO or WT/TgG3 S models, with the WT/TgG3S mice exhibiting the lowest levels of DRG GL-3 storage (FIG. 15B).

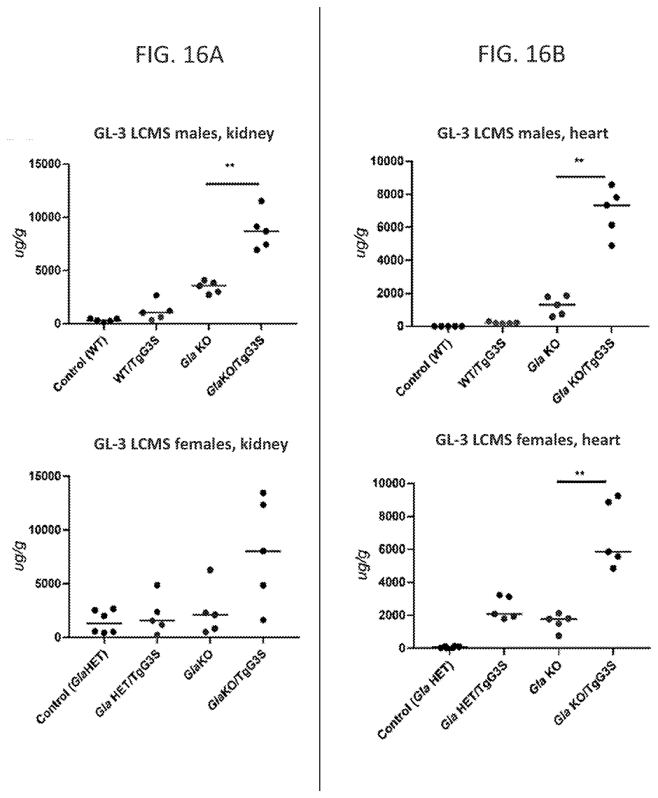

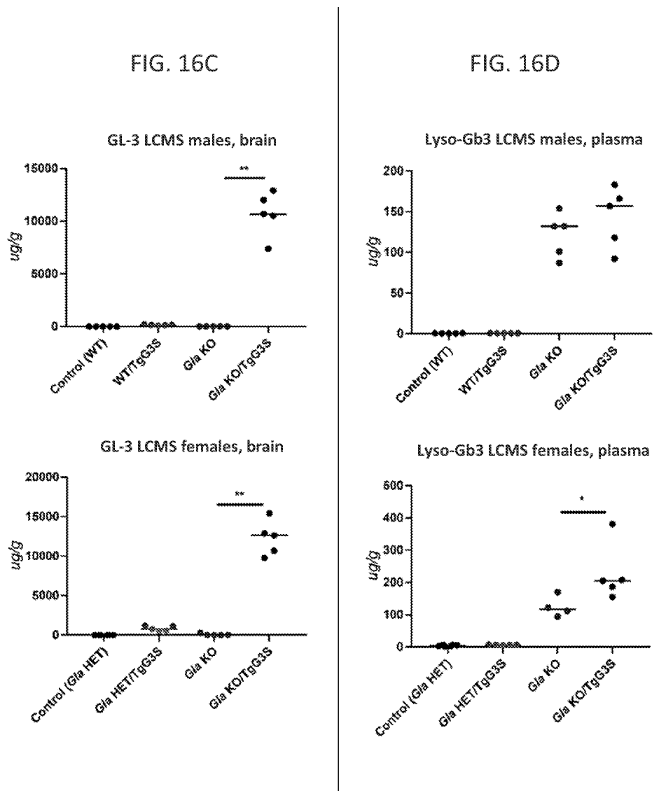

Quantification of substrate (lyso-Gb3 in plasma, GL-3 in tissues) by LC-MS/MS confirmed greater storage of these substrates in the in the kidney, heart, brain, and plasma of aggravated mice (Gla KO/TgG3S) compared to Gla KO mice (FIG. 16A-FIG. 16D). In the kidneys of male wild type mice, GL-3 storage was very low, allowing the slight increase in GL-3 storage in TgG3S mice to be distinguished. Storage of GL-3 in male Gla KO mice was greater than in TgG3S mice, and the aggravated Gla KO/TgG3S mice demonstrated significantly increased GL-3 storage compared to the levels seen in Gla KO mice. Storage of GL-3 in kidney tissue of female mice indicated an apparent trend of increasing GL-3 storage from wild type mice having the lowest levels, followed by TgG3S and Gla KO mice, with Gla KO/TgG3S mice having the highest levels of storage.

In the heart tissue of male animals, there was minimal GL-3 storage in wild type and TgG3S mice. While GLA KO male mice had slightly more GL-3 storage in the heart tissue, the aggravated Gla KO/TgG3S mice had significantly increased levels of substrate storage. In female mice, there was minimal GL-3 storage in the heart tissue of wild type mice, with slightly increased levels in TgG3S and Gla KO mice and significantly raised GL-3 storage observed in Gla KO/TgG3S mice.

In the brain tissue of both male and female mice, GL-3 storage levels were low in wild type, Gla KO, and TgG3S mice. In both sexes, Gla KO/TgG3S mice had significantly increased GL-3 storage in the brain compared to the other three models studied.

In plasma, lyso-Gb3 storage levels were minimal in both wild type and TgG3S mice. These levels were increased to a similar degree in both male Gla KO and Gla KO/TgG3S mice. In female mice, lyso-Gb3 storage levels were increased in Gla KO mice compared to the wild type and TgG3S models; however, there was a significant increase in lyso-Gb3 storage levels between Gla KO and Gla KO/TgG3S mice.

Cumulatively, this natural history study confirms that the aggravated Fabry mouse model overexpressing human Gb3 synthase (Gla KO/TgG3S mice) begins to display disease-relevant abnormalities around 18-25 weeks of age (4.5-6 months of age). Furthermore, Gla KO/TgG3S mice exhibit a generally more severe phenotype than that of the non-aggravated Fabry mouse (Gla KO). Specifically, Gla KO/TgG3S mice display more severe body weight loss (wasting [males and females]) and sensory deficit (increased hotplate latency [males only]) than sex-matched Gla KO mice. Gla KO/TgG3S mice also display progressive renal impairment (increased serum BUN levels, decreased urine osmolality [males and females]), which was not evident in Gla KO mice, in addition to demonstrating greater accumulation of GL-3 in the kidney, heart, DRG, brain, and plasma. The aggravated Fabry mouse model (Gla KO/TgG3S) also demonstrated some secondary lesions of degeneration, necrosis, and mineralization in kidney (mononuclear degenerative interstitial nephritis) and heart (cardiomyocyte necrosis and mineralization) that were never observed in any Gla KO mice and likely explained the more pronounced phenotype. Interestingly, storage material was also seen in kidney glomeruli, including podocytes, similar to Fabry patients and unlike Gla KO mice. Storage in podocytes, the cells that constitute the filtration barrier in the glomeruli, is key to the physiopathology of Fabry disease, and likely accounts for increased proteinuria and decreased urine osmolarity in both the mouse aggravated model and in patients.

The GLA KO/TgG3S mice developed progressive ataxia with severe tremors and ambulatory deficits prompting euthanasia around 35-40 weeks of age unlike Gla KO mice that demonstrate a normal lifespan. This seems to be attributable to marked GL-3 storage in the CNS, including in the cerebellum where degeneration and loss of Purkinje cells was observed on histology. However, Purkinje cell degeneration and ataxia are not a feature of Fabry disease in humans. In the aggravated mouse model, artificial overload of Gla substrate, GL-3, is achieved via overexpression of GL-3 synthase driven by a ubiquitous promoter. Accumulation of GL-3 in the CNS is consecutive to neuronal overexpression of Gb3 S in the absence of GLA in the double mutant Gla KO/TgG3S. Ataxia in mice is therefore directly attributable to widespread and marked Gb3 storage material in the CNS, and it can be mitigated by a gene therapy that will restore GLA levels. For this reason, the monitoring of ataxia and survival in the aggravated mouse model is a relevant biomarker for the mouse even though it is not a translational one.

Collectively, the findings from this study support the use of the aggravated Fabry Gla KO/TgG3 S mouse model as test system for efficacy studies with the following efficacy endpoints: lyso-Gb3 storage in plasma, GL-3 storage in tissues (kidney, heart, DRG, brain), histopathology (kidney, heart, DRG, brain), thermosensory function (hotplate), kidney function (BUN, urine osmolarity), ataxia, and survival.

### Example 4: Evaluation of rAAV Vectors for Delivery of hGLA for Gene Therapy

The aim of this study was to determine the optimal human alpha galactosidase A (hGLA) amino acids sequence for gene therapy. Constructs encoding hGLA variants were tested. For fair comparison, the vectors included the same capsid and promoter, and the WPRE enhancer was also present in all of the expression cassettes.

Two to three month-old Fabry mice (Gla KO) were administered an intravenous (IV) injection of the various AAVhu68.hGLA vectors at one of the following doses: 1×10¹¹ GC (5×10¹² GC/kg—middle dose) or 5×10¹¹ GC (2.5×10¹³ GC/kg—high dose). PBS treated Fabry Gla KO and WT mice served as controls. Blood was collected for serum isolation at 1 week and 3 weeks post injection (pi) and for plasma isolation at 4 weeks pi, the necropsy timepoint. Brain, spinal cord with dorsal root ganglia (DRG), heart, kidney, liver, skin, small intestine, and large intestine were collected 4 weeks pi with half processed for histology, and the other half frozen for biochemical analysis (storage quantification by quantitative mass spectrometry and GLA enzyme activity measurement). The primary efficacy endpoint to compare vectors, was quantification of storage material in target organs. In Gla KO mice, storage material globotriaosylceramide (GL-3) can be stained by immunohistochemistry (IHC) on zinc-formalin paraffin embedded tissue sections. Storage is seen, with progressive worsening with age, as brown deposits in the epithelial cells of kidney tubes. Storage can also be visualized in DRG neurons on H&E stain as enlarged clear stained neurons (their clear color is due to glycolipid storage material in the cytoplasm). Other target organs of Fabry disease such as heart, intestine, or brain vasculature demonstrate low and inconsistent storage staining in the traditional Gla KO mouse model. These organs were collected and processed but did not allow efficacy assessment of the vectors.

The Gla KO mouse is a widely used model for Fabry disease (Ohshima T, Murray G J, Swaim W D, Longenecker G, Quirk J M, Cardarelli C O, Sugimoto Y, Pastan I, Gottesman MM, Brady R O, Kulkarni A B: α-Galactosidase A deficient mice: A model of Fabry disease. Proc Natl Acad Sci 94: 2540-2544, 1997). Hemizygous males display abnormal kidney and liver morphology, both with accumulations of globotriaosylceramide. They also exhibit mild cardiomyopathy and abnormal cardiovascular physiology. The small size, reproducible phenotype, and efficient breeding allow quick studies that are optimal for preclinical in vivo screening of vectors.

The following vectors were compared:
AAVhu68.CB7.hGLAnat.WPRE.rBG
AAVhu68.CB7.hGLAco.WPRE.rBG
AAVhu68.CB7.hGLAco(M51C_G360C).rBG

The IV route was selected due to ease of performance, reproducibility, and robust liver and heart transduction allowing extraction of transgenic GLA for analysis. It is also the intended clinical route of administration. The selected dose range, 1×10¹¹ GC to 5×10¹¹ GC (equivalent to approximately 5×10¹² GC/kg to 2.5×10¹³ GC/kg) was selected to achieve muscle, heart, and liver transduction at the highest dose. The lowest dose was anticipated to be suboptimal and thus to better differentiate efficacy between the different vectors.

Each group included a minimum of 6 mice (males and females) to enable statistical analysis of the pharmacological readouts.

Pharmacological readouts included biochemistry assays (including but not necessarily limited to GLA enzymatic activity, determination of the total amount of enzyme, binding to the mannose-6-phosphate receptor, GL-3 storage), and histology endpoints (GL-3 staining). Antibodies to hGLA were measured.

TABLE
Group designation

| | Number of | | | | |
|---|---|---|---|---|---|
| Treatment | animals | Genotype | Gender | Dose | Route |
| PBS | 6-10 per | Gla KO | Hemizygous | N/A | IV |
| PBS | dose | WT | males and | N/A | |
| AAVhu68.CB7.hGLAcoWPRE.rBG | | Gla KO | KO females | MD: 1 × 10¹¹ | |
| AAVhu68.CB7.hGLAco(M51C_G360C).rBG | | Gla KO | | | |
| AAVhu68.CB7.hGLAnat.WPRE.rBG | | Gla KO | | HD: 5 × 10¹¹ | |

### Results

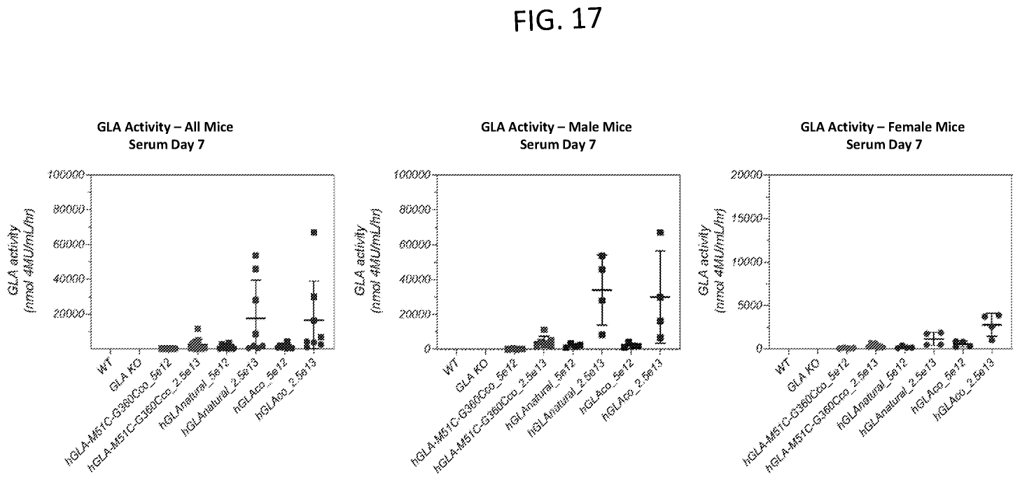

GLA activity levels measured in serum samples acquired 1 week post injection revealed overall, GLA activity levels were dose-dependent, with higher levels observed at the 2.5×10¹³ GC/kg dose with all three vectors (FIG. 17). All three vectors produced higher average levels of GLA activity compared to wild type and GLA KO controls. In the higher dose group (2.5×10¹³ GC/kg), mice administered AAVhu68.hGLAnat and AAVhu68.hGLAco exhibited higher levels of GLA activity compared to mice injected with AAVhu68.hGLAco(M51C_G360C). Male mice demonstrated higher enzyme activity than females, as expected due to more efficient AAV transduction and gene expression in hepatocytes from males compared to female mice, which is a murine specific phenomenon not encountered in nonhuman primates and humans. GLA activity was similar among male mice administered both doses of AAVhu68.hGLAco(M51C_G360C) and the low dose (5.0×10¹² GC/kg) of AAVhu68.hGLAnat and AAVhu68.hGLAco. However, GLA activity levels were much higher in male injected with the high dose (2.5×10¹³ GC/kg) of AAVhu68.hGLAnat and AAVhu68.hGLAco. While GLA activity levels were much lower in female mice, the same trend in GLA activity levels among the three vectors observed in male mice was also seen in female mice.

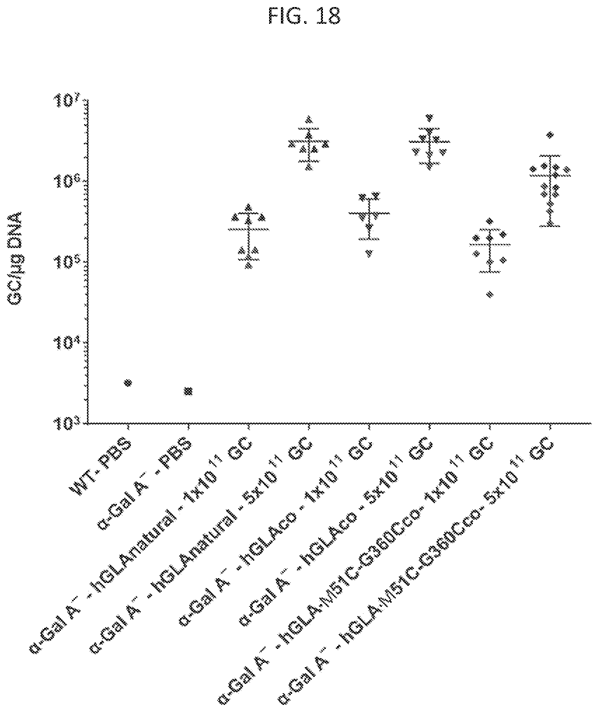

The majority of enzyme activity measured in the serum comes from protein that is being expressed in and secreted from hepatocytes. In order to investigate the cause of lower enzyme activities in serum observed with the vector expressing the engineered candidate AAVhu68.hGLAco(M51C_G360C), we performed a vector genome biodistribution analysis in liver samples collected at necropsy. For all three vectors, mice administered the higher dose (2.5×10¹³ GC/kg) demonstrated higher rates of transduction than those injected with the corresponding lower dose of each vector, and the 3 different vectors yielded similar levels of vector genomes at a given dose level This indicates that decreased enzyme activity with the vector encoding the engineered candidate is attributable to decreased expression of the transgene (FIG. 18).

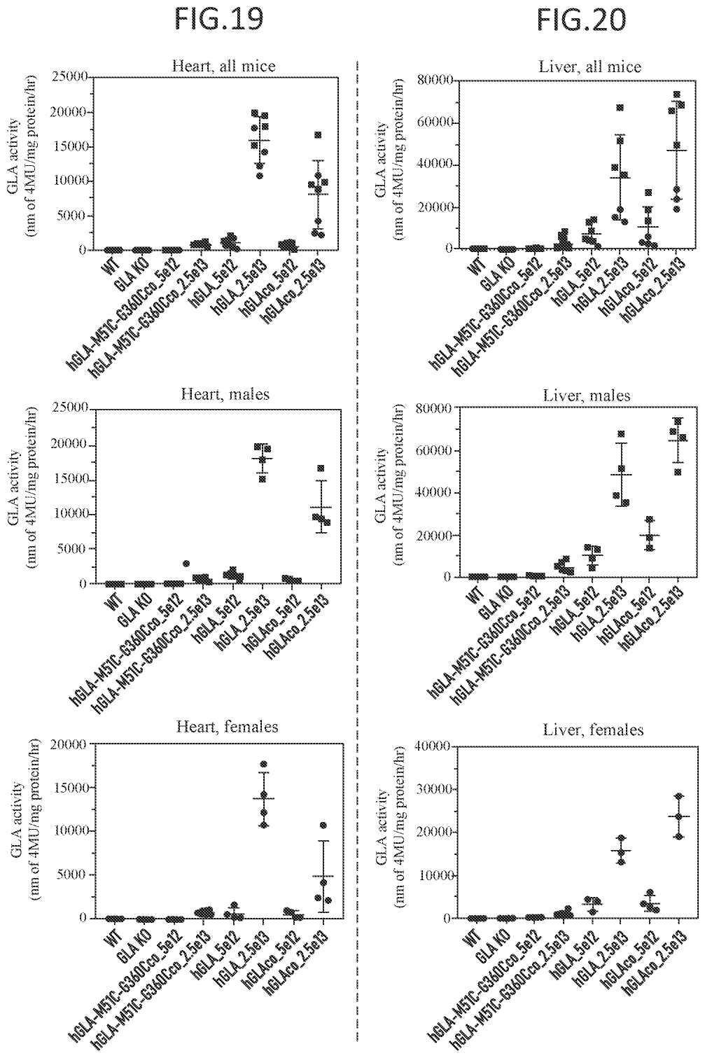

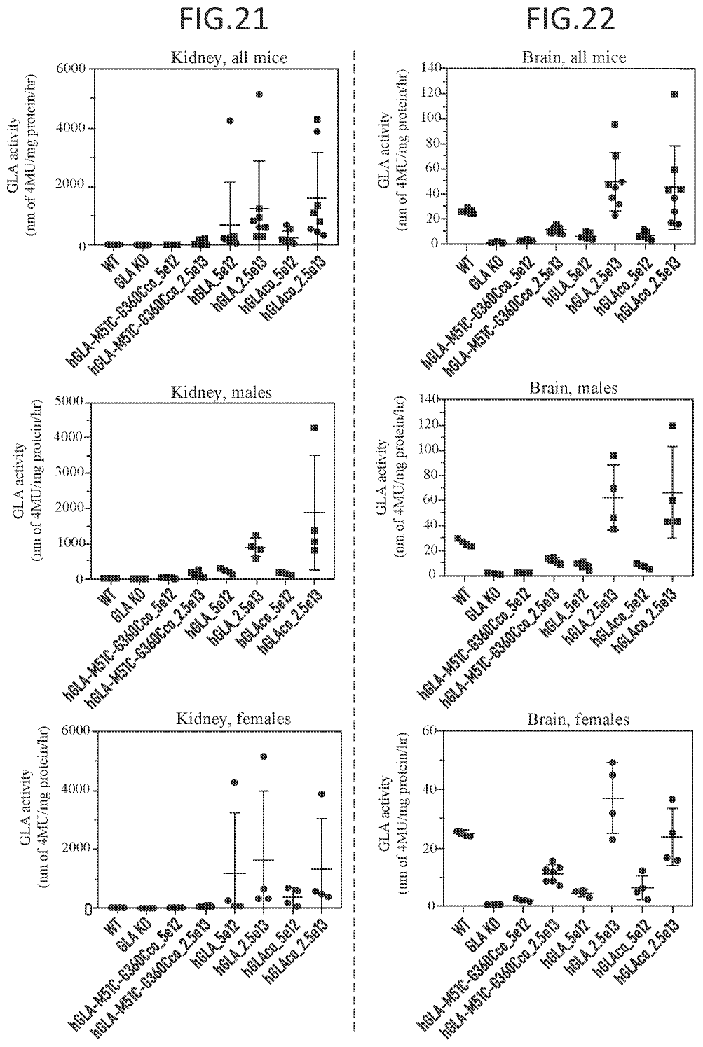

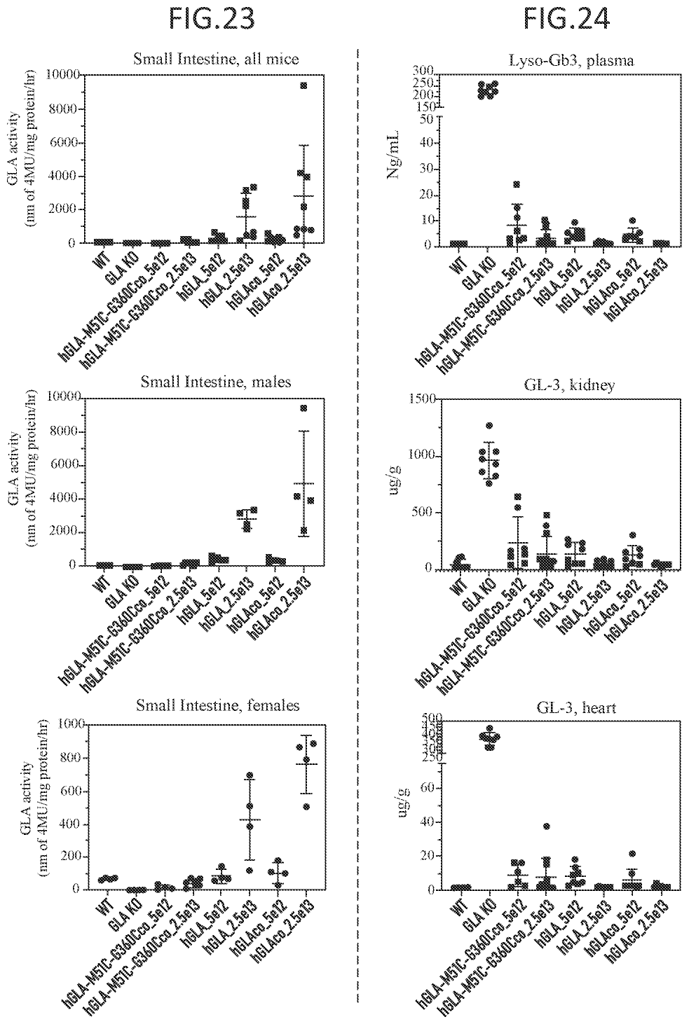

Tissue enzyme activity levels were also analyzed in disease related organs at Day 28 post IV vector injection of 5.0×10¹² or 2.5×10¹³ GC/kg dosage. The figures below show enzyme activity results of heart (FIG. 19), liver (FIG. 20), kidney (FIG. 21), brain (FIG. 22), and small intestine (FIG. 23). Overall GLA activity levels measured in heart tissue were highest in mice administered the high dose (2.5×10¹³ GC/kg) of AAVhu68.hGLAnat and AAVhu68.hGLAco. Much lower GLA activity levels were observed in the mice administered the low dose (5.0×10¹² GC/kg) of AAVhu68.hGLAnat and AAVhu68.hGLAco and both doses of AAVhu68.hGLAco(M51C_G360C). Similar activity levels were seen in both male and female mice.

In the liver, overall GLA activity was higher in mice administered the high dose (2.5×10¹³ GC/kg) of all three AAVhu68.hGLA and was highest in those treated with AAVhu68.hGLAnat and AAVhu68.hGLAco. Generally, GLA activity was slightly higher in male mice compared to female mice.

While there was less dose-dependent variation of overall GLA activity measured in kidney tissue, activity levels still tended to be higher in mice administered the high dose (2.5×10¹³ GC/kg) of all three AAVhu68.hGLA. There was no significant difference in GLA activity levels observed between male and female mice; however, there was much more variability in GLA activity levels measured in female mice.

Significant levels of GLA activity were observed in the brain tissue of wild type mice. Once again, GLA activity levels were higher among mice administered the high dose (2.5×10¹³ GC/kg) of all three AAVhu68.hGLA and highest in those treated with the high dose (2.5×10¹³ GC/kg) of AAVhu68.hGLAnat and AAVhu68.hGLAco. Similar activity levels were seen in both male and female mice.

GLA activity levels in the small intestine of mice treated with both doses of AAVhu68.hGLAco(M51C_G360C) and the low dose (5.0×10¹² GC/kg) of AAVhu68.hGLAnat and AAVhu68.hGLAco were low and similar in magnitude. The highest levels of GLA activity were seen in mice high dose (2.5×10¹³ GC/kg) of AAVhu68.hGLAnat and AAVhu68.hGLAco. No significant variation was seen between male and female mice.

In summary, consistent with the serum results above, tissue levels of GLA enzyme activity were dose dependent, comparable between the two candidates encoding the unmodified natural protein (engineered sequence or not), and markedly lower in the candidate encoding the engineered protein hGLAco(M51C_G360C). No gender effect was seen in organs other than liver.

To further evaluate the pharmacology of the three vectors, we measured the amount of storage material lyso-Gb3 by LC-MS/MS in the plasma and GL-3 in tissues from GLA KO mice 28 days post AAV administration and compared these levels with those measured in PBS-treated GLA KO and wild type control mice. Lyso-Gb3 and GL-3 storage reduction was consistent with enzyme activity levels; the two vectors encoding GLA (AAVhu68.hGLAnat and AAVhu68.hGLAco) led to full storage elimination at the high dose (2.5×10¹³ GC/kg) in plasma, kidney, and heart samples (FIG. 24). However, the vector encoding hGLAco(M51C_G360C) only partially reduced the levels of lyso-Gb3 storage in plasma and GL-3 storage in kidney and heart tissue.

### Example 5: Evaluation of rAAV Vectors for Delivery of hGLA for Gene Therapy

The aim of this study was to evaluate three vectors at up to three different doses (2.5×10¹² GC/kg, 5×10¹² GC/kg, 2.5×10¹³ GC/kg) to determine efficacy in Gla KO mice following IV administration. All vectors evaluated had the same capsid, promoter, and polyA signal, but included a different version of the human GLA transgene. The three transgenes evaluated were hGALco (same as in Example 4), hGLAco(M51C_G360C) (same as in Example 4), and hGLA-D233C-I359Cco. The hGLAco(M51C_G360C) transgene encodes an engineered GLA protein with two point mutations introducing a disulfide bound stabilizing the enzyme in its active dimer form. The hGLAco(D233C_I359C) transgene encodes a second version of engineered GLA protein with two point mutations introducing a disulfide bound stabilizing the enzyme in its active dimer form.

Adult mice (3.5 to 4.5 months of age) received a single IV administration of 1 of the 3 candidate vectors (AAVhu68.hGLAco, AAVhu68.hGLAco(M51C_G360C), or AAVhu68.hGLAco(D233C_I359C) at a low dose of 2.5×10¹² GC/kg, a mid-dose of 5.0×10¹² GC/kg, or a high dose of 2.5×10¹³ GC/kg (AAVhu68.hGLAco(D233C_I359C) only). Vehicle (PBS)-treated WT and Gla KO mice served as controls.

| Vector ID | Construct | Doses Evaluated^{ba} |
|---|---|---|
| WTco | AAVhu68.CB7.hGLAco.rBG | Low dose: 2.5 × 10¹² GC/kg |
| | | Mid-dose: 5.0 × 10¹² GC/kg |
| AT#1 | AAVhu68.CB7.hGLAco(M51C_G360C).rBG | Low dose: 2.5 × 10¹² GC/kg |
| | | Mid-dose: 5.0 × 10¹² GC/kg |
| AT#2 | AAVhu68.CB7.hGLAco(D233C_I359C).rBG | Low dose: 2.5 × 10¹² GC/kg |
| | | Mid-dose: 5.0 × 10¹² GC/kg |
| | | High dose: 2.5 × 10¹³ GC/kg |

Animals were monitored daily for viability. Serum was collected on Day 7 for evaluation of transgene product expression (GLA enzyme activity). On Day 28, necropsies were performed, and the heart, kidney, liver, and spinal cord with DRG were collected and processed for histology, GL-3 quantification, and evaluation of GLA enzyme activity. Plasma was also collected for lyso-Gb3 quantification and evaluation of GLA enzyme activity.

Intravenous administration was well-tolerated for all vectors evaluated. All animals survived to the scheduled necropsy time point.

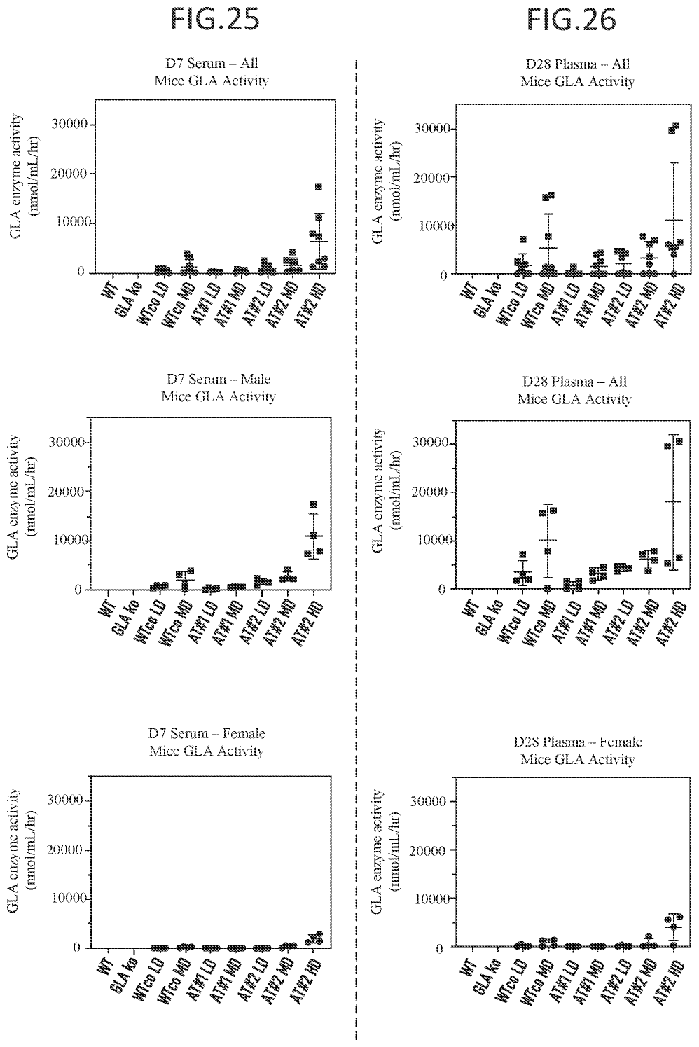

In the aggregated data for both male and female Gla KO mice, serum transgene product expression (GLA enzyme activity) was greatest in Gla KO mice administered the high dose (2.5×10¹³ GC/kg) of AAVhu68.hGLAco(D233C_I359C). GLA enzyme activity levels were similar among Gla KO mice in the remaining treatment groups, although there was a slight dose-dependent response in Gla KO mice administered AAVhu68.hGLAco or AAVhu68.hGLAco(D233C_I359C). Male Gla KO mice demonstrated higher GLA enzyme activity than females. GLA enzyme activity was greatest in male Gla KO mice administered the high dose (2.5×10¹³ GC/kg) of AAVhu68.hGLAco(D233C_I359C), and there was some dose-dependency of GLA enzyme activity in male Gla KO mice administered AAVhu68.hGLAco or AAVhu68.hGLAco(D233C_I359C). GLA enzyme activity in female Gla KO mice was very low, with the highest levels observed in those administered the high dose (2.5×10¹³ GC/kg) of AAVhu68.hGLAco(D233C_I359C) (FIG. 25).

Aggregated data for transgene product expression (GLA enzyme activity) measured in plasma collected 28 days after administration revealed an apparent dose-dependent effect for all 3 AAV vectors studied, with the greatest levels of GLA enzyme activity observed in Gla KO mice administered the mid-dose (5.0×10¹² GC/kg) of AAVhu68.hGLAco and the high dose (2.5×10¹³ GC/kg) of AAVhu68.hGLAco(D233C_I359C). GLA enzyme activity was much higher in male Gla KO mice than in female Gla KO mice. A dose-dependent effect of AAVhu68.hGLA on GLA activity was observed for all test articles in male Gla KO mice, with the mid-dose (5.0×10¹² GC/kg) of AAVhu68.hGLAco and the high dose (2.5×10¹³ GC/kg) of AAVhu68.hGLAco(D233C_I359C) producing the highest enzyme activity. GLA activity levels were universally lower in female Gla KO mice, with the high dose (2.5×10¹³ GC/kg) of AAVhu68.hGLAco(D233C_I359C) affording the highest level of activity (FIG. 26).

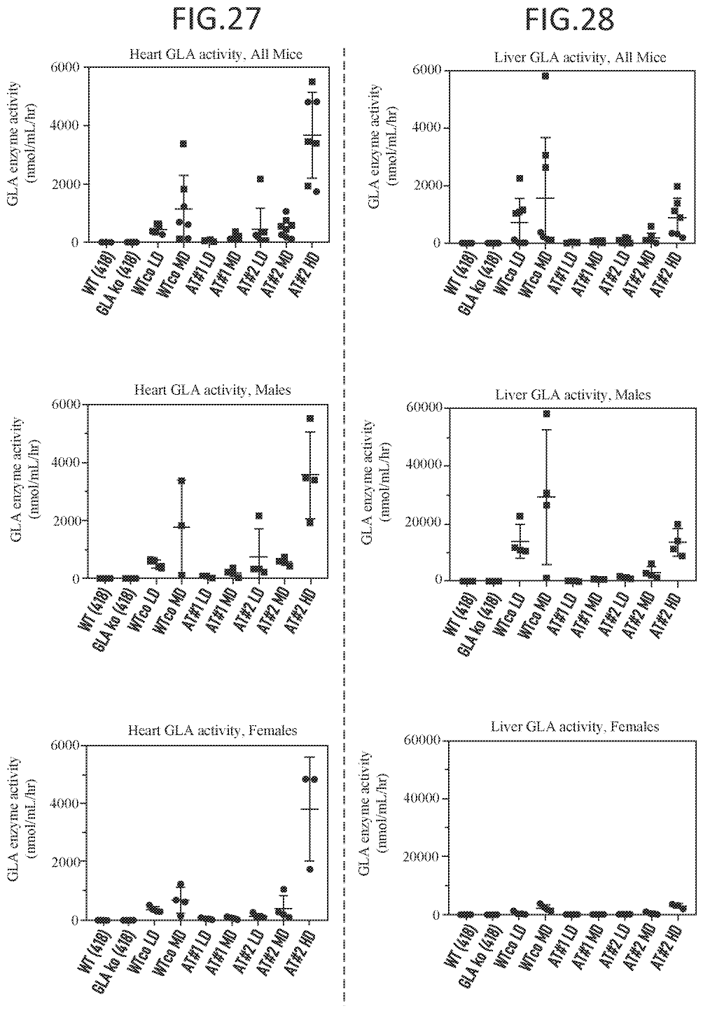

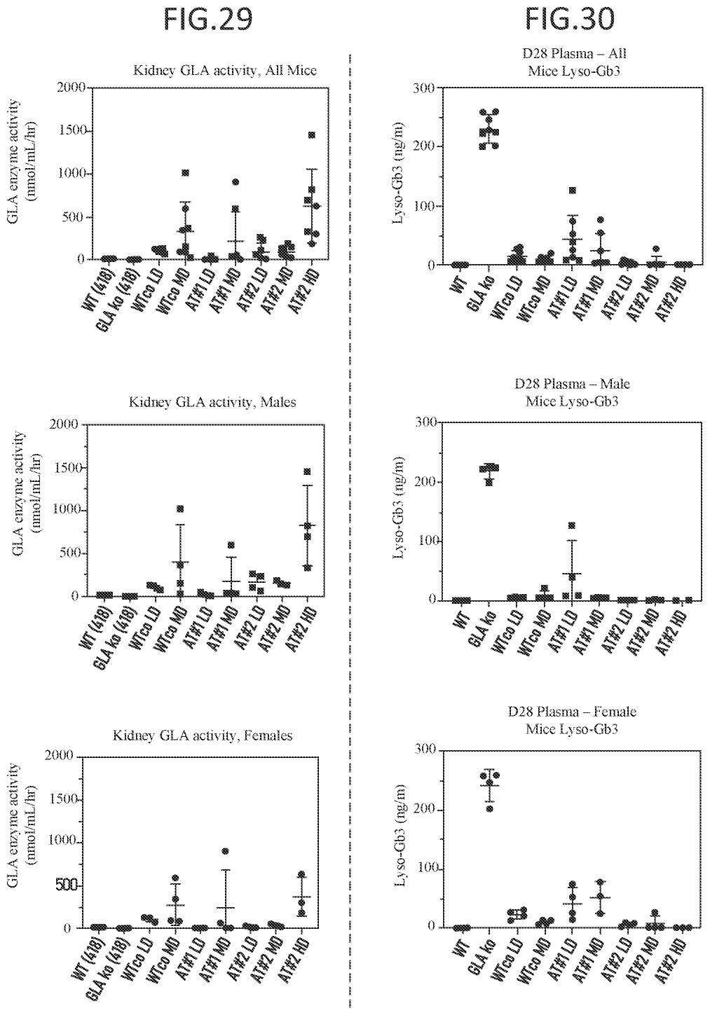

GLA enzyme activity in heart, liver, and kidney tissue is shown in FIG. 27, FIG. 28, and FIG. 29, respectively.

Aggregated data for GLA enzyme activity in heart tissue showed an apparent dose-dependent effect for all 3 AAV vectors studied, with the greatest levels of GLA enzyme activity observed in Gla KO mice administered the mid-dose (5.0×10¹² GC/kg) of AAVhu68.hGLAco and the high dose (2.5×10¹³ GC/kg) of AAVhu68.hGLAco(D233C_I359C). Similar levels of GLA enzyme activity were observed in male and female Gla KO mice.

Combined data for GLA enzyme activity in liver samples revealed the highest levels of enzyme activity in Gla KO mice administered both doses of AAVhu68.hGLAco and the high dose (2.5×10¹³ GC/kg) of AAVhu68.hGLAco(D233C_I359C). These observations were mirrored in the results from male Gla KO mice. Female Gla KO mice had significantly lower levels of GLA enzyme activity than male Gla KO mice and demonstrated less variation in activity among the three AAV vectors and doses.

For both male and female Gla KO mice, GLA enzyme activity measured in kidney samples showed an apparent dose-dependent effect for all 3 AAVhu68.hGLA vectors studied, with the highest levels of activity in the mice administered the mid-dose (5.0×10¹² GC/kg) of AAVhu68.hGLAco and AAVhu68.hGLAco(M51C_G360C) and the high dose (2.5×10¹³ GC/kg) of AAVhu68.hGLAco(D233C_I359C). These trends were mirrored when GLA enzyme activity was analyzed by sex, which also revealed similar GLA enzyme activity levels in male and female Gla KO mice.

Plasma from treated Gla KO mice was evaluated to assess efficacy of the vectors at decreasing lyso-Gb3 storage (FIG. 30). The data revealed that treatment of Gla KO mice with the mid-dose (5.0×10¹² GC/kg) of AAVhu68.hGLAco and both mid-dose and high dose (5.0×10¹² GC/kg and 2.5×10¹³ GC/kg, respectively) of AAVhu68.hGLAco(D233C_I359C) fully cleared lyso-Gb3 storage in plasma. These results were consistent in both male and female Gla KO mice.

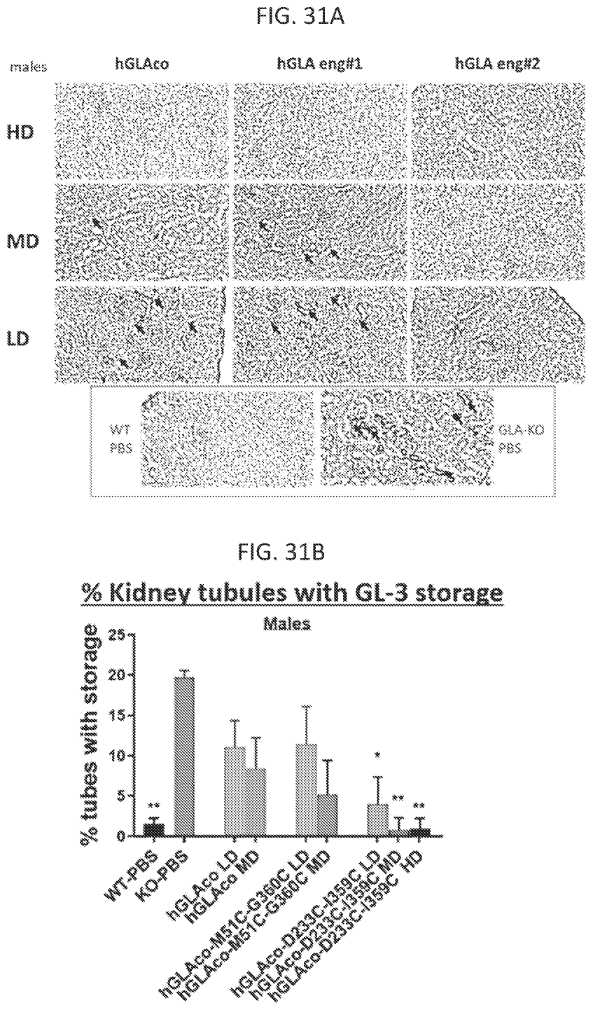

Immunohistochemistry data from kidney tissue samples revealed that while some reduction in GL-3 storage was observed with the highest doses of all three vectors administered, the most apparent reduction of GL-3 storage was observed in Gla KO mice treated with the high dose (2.5×10¹³ GC/kg) of AAVhu68.hGLAco(D233C_I359C) (FIG. 31A). Consistent with previous results, quantification of GL-3 storage from IHC staining of kidneys revealed that Gla KO mice treated with all 3 doses of AAVhu68.hGLAco(D233C_I359C) had significantly less kidney GL-3 storage in tubules compared to the vehicle-treated Gla KO controls. None of the mice treated with AAVhu68.hGLAco or the other engineered variant AAVhu68.hGLAco(M51C_G360C) had significant storage reductions. This reduction in GL-3 storage was observed to be dose-dependent, with the greatest effect in Gla KO mice administered the highest dose (2.5×10¹³ GC/kg) of AAVhu68.hGLAco(D233C_I359C) (FIG. 31B).

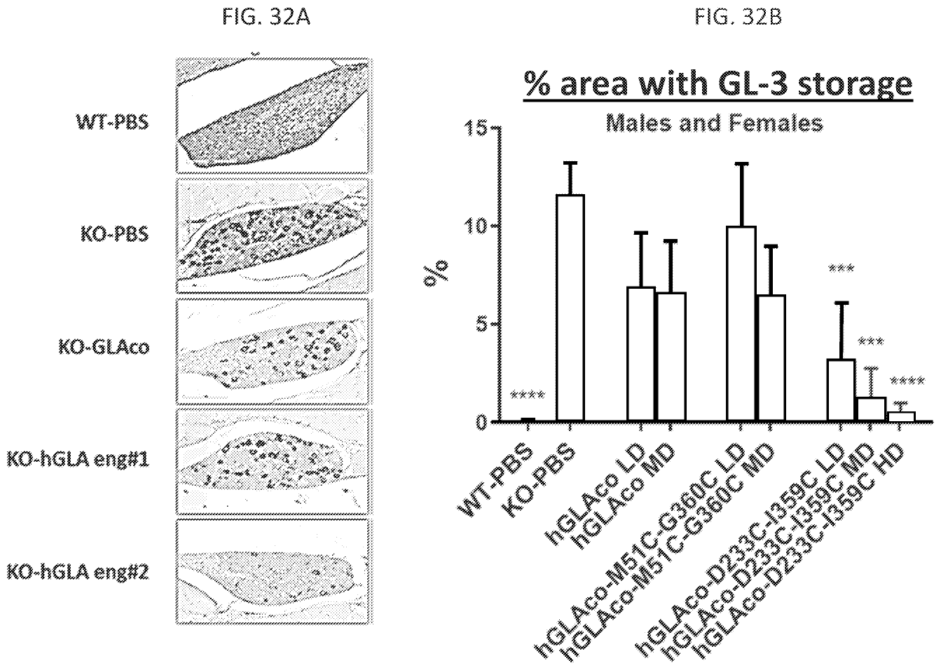

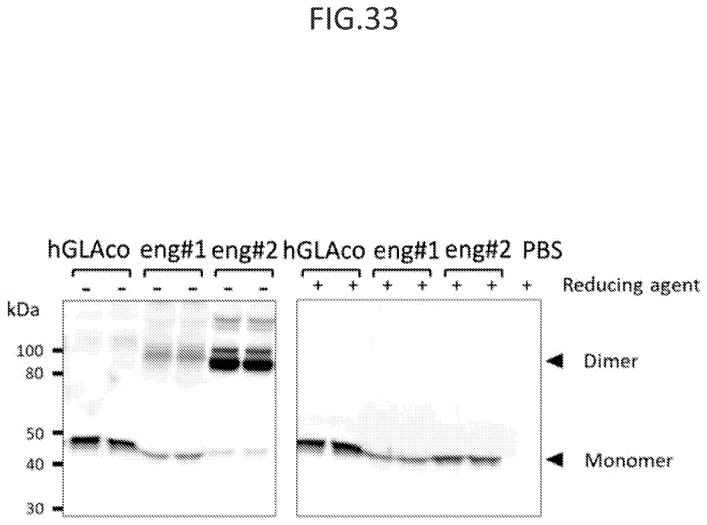

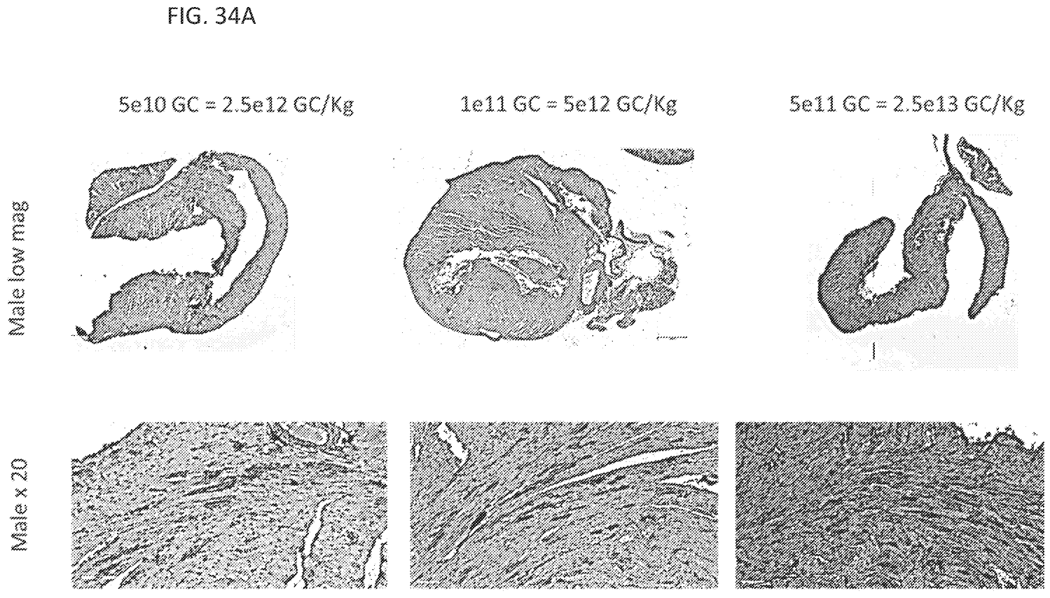

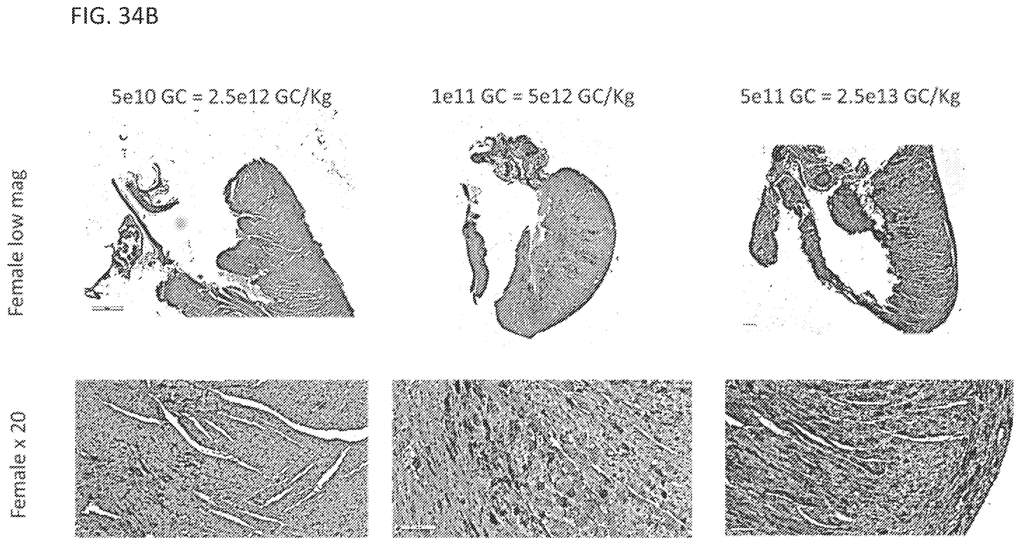

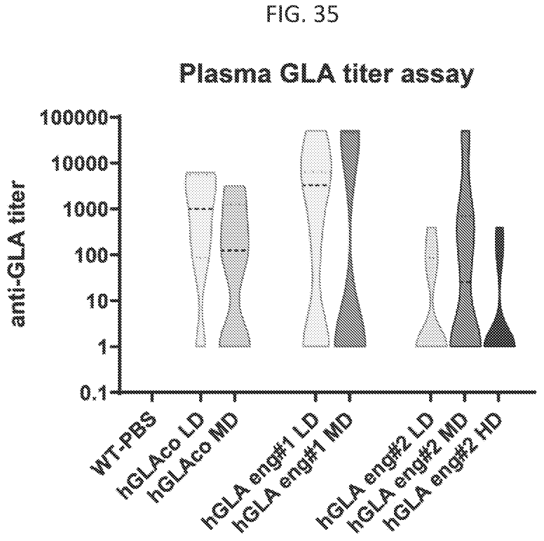

Immunohistochemistry data from longitudinal sections of DRG revealed that Gla KO mice treated with AAVhu68.hGLAco(D233C_I359C) had the least GL-3 storage compared to Gla KO mice treated with vehicle, AAVhu68.hGLAco, or AAVhu68.hGLAco(M51C_G360C) (FIG. 32A). Quantification of these IHC data revealed that DRG neuronal GL-3 storage was significantly reduced in Gla KO mice treated with all three doses of AAVhu68.hGLAco(D233C_I359C) compared to vehicle-treated Gla KO mice. This response was observed to be dose-dependent, having the greatest effect in mice administered the highest dose (2.5×10¹³ GC/kg) of AAVhu68.hGLAco(D233C_I359C). Once again, the other candidates, AAVhu68.hGLAco and the other engineered variant AAVhu68.hGLAco(M51C_G360C), did not lead to significant GL-3 storage reduction in DRG (FIG. 32B).

Cumulatively, AAVhu68.hGLAco(D233C_I359C) administration to Gla KO mice led to significant transgene product expression (GLA enzyme activity) with the highest plasma and tissue in vivo efficacy compared to the other vectors evaluated. AAVhu68.hGLAco(D233C_I359C)-treated Gla KO mice exhibited significant dose-dependent reductions in kidney and DRG GL-3 storage compared to vehicle-treated Gla KO mice, whereas administration of the non-engineered AAVhu68.hGLAco vector did not significantly reduce GL-3 storage at the same dose levels.

### Example 6: Evaluation of AAVhu68.hGLAco(D233C_I359C) in Non-Human Primates

This study was designed to evaluate preliminary pharmacology and safety of AAVhu68.hGLAco(D233C_I359C) administered intravenously to cynomolgus macaques.

Adult NHPs (N=4) received a single IV dose of AAVhu68.hGLAco(D233C_I359C) at a dose of 2.5×10¹³ GC/kg. In-life evaluations included clinical observations performed daily, body weights, clinical pathology of the blood (CBC, coagulation panel, serum chemistry [including the cardiac biomarker troponin I]), and cardiac function assessment (EKG and echocardiography). On Day 60±3 post vector administration, all animals were necropsied. At necropsy, tissues were collected for histopathological examination. Target tissues were collected for vector biodistribution analysis and comprehensive histological evaluation of transgene product expression localization (human GLA ISH [mRNA] and human GLA IHC [protein]). PBMC, splenocytes, and liver lymphocytes were also collected to measure T cell responses to the capsid and transgene product (IFN-γ ELIspot). A study design is provided in the table below.

| | Study Day | | | | | | |
|---|---|---|---|---|---|---|---|
| | | Day | Day | Day | Day | Day | Day |
| | BL^{a} | 0 | 3 | 7 ± 1 | 14 ± 1 | 28 ± 2 | 60 ± 3 |
| Dosing - IV administration of | | X | | | | | |
| AAVhu68.CB7.CI.hGLAco | | | | | | | |
| (D233C_I359C).WPRE.rBG (N = 4) | | | | | | | |
| Clinical observations | Daily | | | | | | |
| Body weight, temperature, respiratory rate, | X | X | X | X | X | X | X |
| heart rate | | | | | | | |
| EKG and electrocardiogramhy | X | | | | | | X |
| Biomarker (plasma) - Transgene product | | X | X | X | X | X | X |
| expression, | | | | | | | |
| Anti-transgene product antibodies (ADAs) | | | | | | | |
| Clinical Pathology - Blood (CBC, serum | X | X | X | X | X | X | X |
| clinical chemistry, coagulation)^{b} | | | | | | | |
| Immunology - NAbs against capsid^{c} | X | | | | | | X |
| Immunology - T cell response to capsid or | X | | | | | | X |
| transgene product (PBMCs) | | | | | | | |
| Necropsy and sample collection^{d} | | | | | | | X |

^{a}BL was up to 21 days prior to dosing.
^{b}Serum chemistry analysis included troponin I (cardiac biomarker).
^{c}Samples were collected and stored for future analysis.
^{d}Tissues were collected for histopathology, vector biodistribution analysis, and histological evaluation of transgene product expression (human GLA ISH staining [mRNA] and human GLA IHC staining [protein]). Liver and spleen lymphocytes were collected for measurement of T cell responses to the capsid or transgene product (IFN-γ ELISpot).
Abbreviations:
ADA, anti-drug antibodies;
BL, baseline;
ELISpot, enzyme-linked immunosorbent spot;
GLA, α-galactosidase;
IFN-γ, interferon gamma;
IHC, immunohistochemistry;
IN, intranasal;
ISH, in situ hybridization;
mRNA, messenger ribonucleic acid;
MS, mass spectrometry;
NAbs, neutralizing antibodies;
PBMCs, peripheral blood mononuclear cells

### Baseline Sample Collection:

Baseline blood samples, including complete blood count (CBC), coagulation, cardiac biomarkers, serum chemistry, and PBMC/ELISPOT are collected from all animals up to 21 days prior to dosing test or reference article (baseline), and at time points indicated in the table below. Prior to sample collection vitals (i.e., temperature, heart rate, respiration) are obtained from each animal.
a) Capsid neutralizing antibodies (serum): Blood (up to 2 mL) for testing the presence of AAVhu68 NAb is collected at baseline and D60 (necropsy). Blood is collected via red top tubes (with or without serum separator), allowed to clot, and centrifuged.
b) Cardiac biomarkers (serum): Blood (up to 2 mL) for testing the presence of cardiac toxicity markers (troponin I) is collected at Baseline, D3, D7, D14, D28, and D60 (necropsy). Blood is collected via red top tubes (with or without serum separator), allowed to clot, and centrifuged. Serum is isolated.
c) Transgene expression, antibodies, complement factors or cytokines (plasma): Blood (at least 3 mL) for testing the presence of hGLA, anti-hGLA Ab, and/or complement activation or cytokines (in case of toxicity) is collected at DO, D3, D7, D14, D28, and D60 (necropsy). Blood is collected in labeled lavender top (EDTA K2) and centrifuged within 30 minutes after collection in an approximately +4° C. centrifuge at −2700 RPM (1300±100×g) for 15 minutes.
d) PBMC/ELISPOT: Blood (5 to 10 mL) is collected into sodium heparin (green top tubes) and PBMCs are isolated. Samples are collected at baseline and necropsy. T cell responses to capsid and/or transgene are assessed.
e) Hematology (Cell Counts and Differentials): Blood (up to 2 mL) for complete blood counts with differentials and platelet count is collected. The following parameters are analyzed at the specific time points indicated in the Study Design:
   Red Blood Cell Count
   Hemoglobin
   Hematocrit
   Mean Corpuscular Volume (MCV)
   Mean Corpuscular Hemoglobin (MCH)
   Mean Corpuscular Hemoglobin Concentration (MCHC)
   Platelet Count
   Leukocyte Count
   Leukocyte Differential
   Red blood cell morphology (pathologist review)
   Reticulocyte Count
f) Clinical Chemistry: Blood (up to 2.0 mL) for clinical chemistry studies is collected in labeled red top (serum) tubes, allowed to clot for up to 15 minutes, and centrifuged. The serum is separated and put into labeled microcentrifuge tubes. The following parameters are analyzed at the specific time points indicated in the Study Design:
   Alkaline Phosphatase
   Hemolysis markers: Bilirubin (direct, indirect)
   Creatinine
   Gamma-Glutamyl Transpeptidase
   Glucose
   Serum Alanine Aminotransferase
   Serum Aspartate Aminotransferase
   Albumin
   Albumin/Globulin ratio (calculated)
   Blood Urea Nitrogen
g) Coagulation: Blood (2.0 mL) for coagulation panel is collected in labeled blue top (citrate) tubes. The following parameters are analyzed at the specific time points indicated in the Study Design:
   PTT
   PT
   Fibrinogen
   D-dimers
   Fibrin degradation products

### Cardiac Monitoring

Studies involve a baseline echocardiogram before vector dosing, and an additional echo at study termination. Minimum parameters evaluated include diastolic/systolic volume, stroke volume, cardiac output, fractional shortening, septum thickness, ejection time. The apical two or four chamber, right parasternal long axis four chamber view, right parasternal short axis views are also assessed for ejection fraction, which when combined with heart rate yield left ventricular ejection fraction, end diastolic volume, end systolic volume, stroke volume, and cardiac output.

### Results:

Intravenous administration was well-tolerated based on clinical observations, and all animals survived to the scheduled necropsy time point. Troponin I levels, which can indicate cardiac cell injury, were below the reportable range of 0.200 μg/L to 180 μg/L for all animals at baseline, Day 14, Day 28, and Day 60 with no abnormalities noted on ECG and electrocardiography.

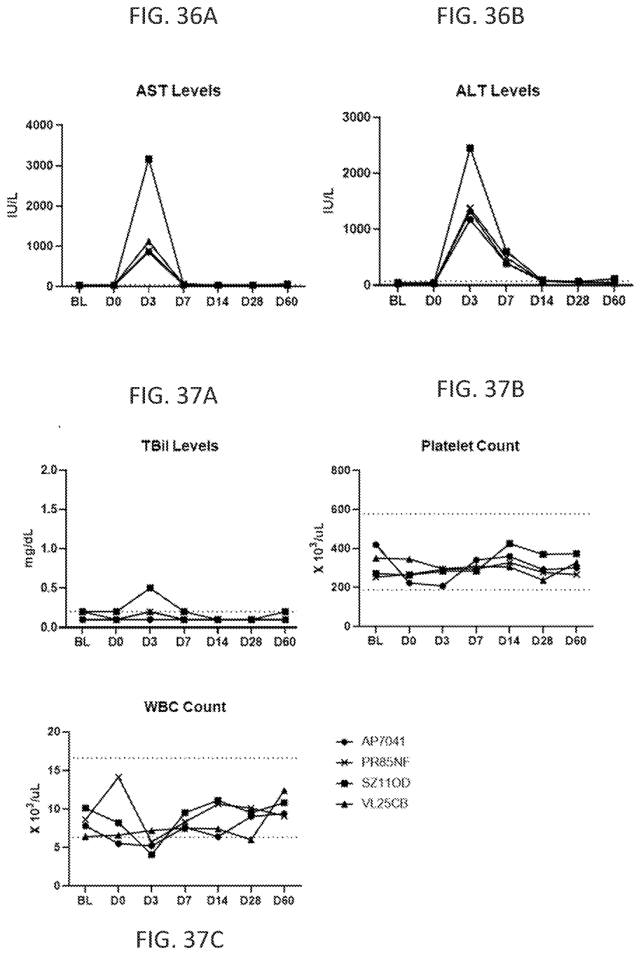

On blood clinical pathology, findings included a transient elevation in AST and ALT levels in all animals at Day 3 and resolved without intervention by Day 7 to Day 14 (FIG. 36A and FIG. 36B).

Total bilirubin (TBil) levels, platelet count, and white blood cell (WBC) count remained within normal limits for all animals throughout the study (FIG. 37A, FIG. 37B, and FIG. 37C).

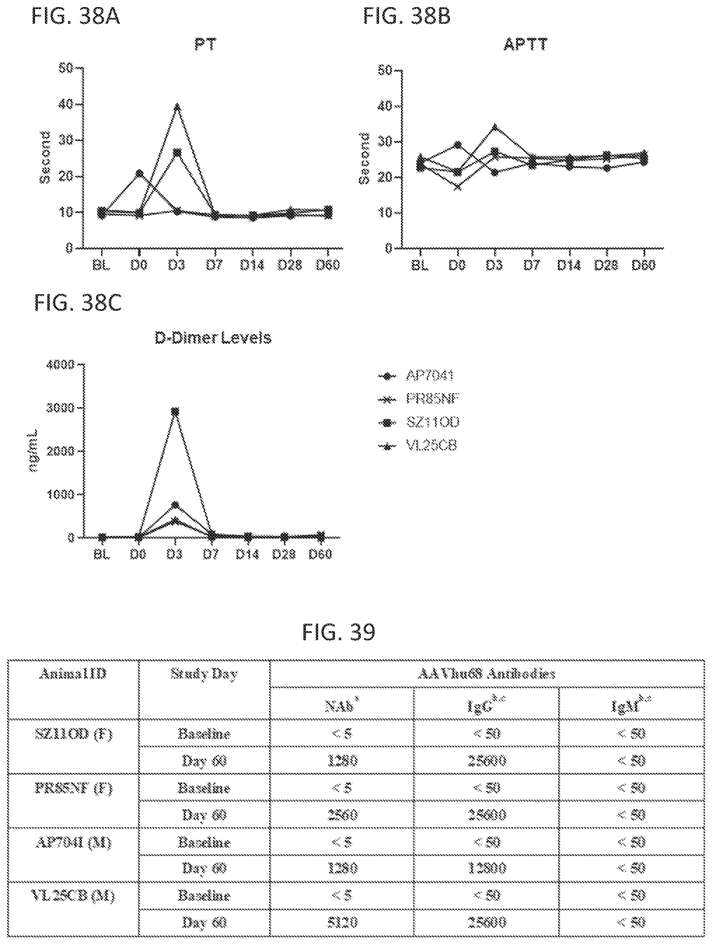

Coagulation data collected from the animals throughout the study revealed transient elevations in prothrombin time (PT), activated APTT, and D-dimer levels in several animals at Day 3 (FIG. 38A, FIG. 38B, and FIG. 38C). These transient elevations were resolved without intervention.

No T-cell responses to the human GLA transgene product or AAVhu68 capsid were observed in PBMCs or lymphocytes from the spleen, cardiac lymph nodes, or liver from animals administered a single IV dose of AAVhu68.hGLAco(D233C_I359C) by IFN-γ ELISpot for any of the peptide pools evaluated.

Histopathological examination of tissues collected at necropsy revealed the presence of some minimal (Grade 1) inflammatory cell infiltrates in some organs with a similar incidence and severity as typical background findings from historical controls and from published literature on background findings. DRG and TRG neuronal degeneration and spinal cord dorsal axonopathy were absent or minimal.

TABLE
Semi-Quantitative Histopathologic Scoring Assessment of Tissues
Collected from Adult NHPs 60 Days Following a Single Intravenous Administration
of AAVhu68.hGLAco(D233C_I359C) (2.5 × 10¹³ GC/kg)

| | Grade of Finding^{a} | | | |
|---|---|---|---|---|
| | Animal | Animal | Animal | Animal |
| Organ (Finding) | SZ11OD (F) | PR85NF (F) | VL25CB (M) | AP704I (M) |
| Brain (intimal | 1 | 0 | 0 | 1 |
| thickening, artery) | (Brain Section 2) | | | (Brain Section 5) |
| Heart, right (infiltrate) | 0 | 1 | 1 | 1 |
| Heart, left (infiltrate) | 0 | 1 | 1 | 1 |
| Heart, septum (infiltrate) | 0 | 1 | 1 | 0 |
| Liver (infiltrate) | 1 | 1 | 0 | 0 |
| Kidney, left (infiltrate) | 0 | 0 | 1 | 0 |
| Kidney, right (infiltrate) | 0 | 0 | 1 | 0 |
| Spinal cord, C (dorsal | 0 | 0.25 | 0 | 0.33 |
| axonopathy) | | (G1 in ¼) | | (G1 in ⅓) |
| Spinal cord, T (dorsal | 0 | 0 | 0.40 | 0 |
| axonopathy) | | | (G1 in ⅖) | |
| Spinal cord, L (dorsal | 0 | 0 | 0 | 0 |
| axonopathy) | | | | |
| DRG, C (neuronal | 0.50 | 0.44 | 0.67 | 0 |
| degeneration) | (G1 in ½) | (G1 in 4/9) | (G1 in ⅔) | |
| DRG, T (neuronal | 0.17 | 0.25 | 0.50 | 1 |
| degeneration) | (G1 in ⅙) | (G1 in ¼) | (G1 in 2/4) | (G1 in 6/6) |
| DRG, L (neuronal | 0 | 0.40 | 0.25 | 1 |
| degeneration) | | (G1 in ⅖) | (G1 in ¼) | (G1 in 2/2) |
| Quadriceps muscle | 1 | 0 | 1 | 0 |
| (infiltrate) | | | | |
| TRG (neuronal | 1 | 1 | 1 | 1 |
| degeneration) | | | | |
| Pituitary gland | 1 | 0 | 0 | 0 |
| (infiltrate) | | | | |
| Median nerve | 0 | 0 | 0 | 1 |
| (axonopathy) | | | | |
| Sciatic nerve | 0 | 0 | 0 | 1 |
| (axonopathy) | | | | |

^{a}Numbers indicate semi-quantitative grading of the finding. 0 = within normal limits, 1 = minimal severity. Grades of less than 1 for spinal cord and DRG were determined by adding the number of grade 1 findings observed and dividing by the total sections evaluated for that tissue (calculation provided in parentheses).

Neutralizing antibodies and non-neutralizing binding antibodies (BAbs) (ie, immunoglobulin G [IgG] and immunoglobulin M [IgM]) against the AAVhu68 capsid were not present at detectable levels in any of the NHPs at baseline, consistent with screening of NAb-negative animals for this study (FIG. 39). As expected, by Day 60, all animals had detectable levels of NAbs and IgG BAbs against the AAVhu68 capsid, while IgM BAbs against the AAVhu68 capsid remained below the detectable limit.

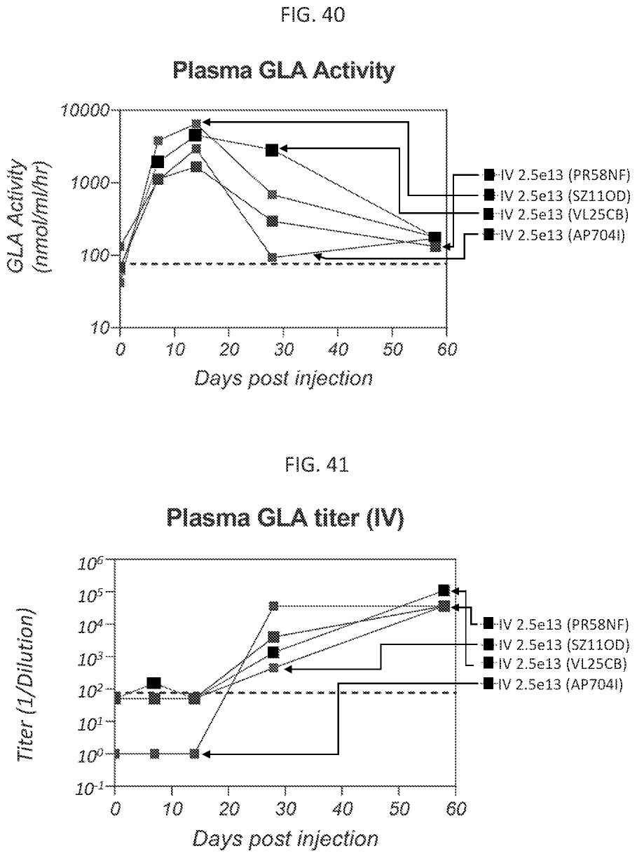

In plasma, IV administration of AAVhu68.hGLAco(D233C_I359C) led to significant levels of transgene product expression (GLA enzyme activity). Average GLA enzyme activity increased approximately 50-fold from Day 0 to Day 14 post administration (FIG. 40). GLA enzyme activity levels peaked at Day 14 and then decreased through Day 60. By the final time point evaluated (Day 60), GLA enzyme activity was approximately 2-fold higher than baseline levels observed on Day 0. This decline in transgene product expression after Day 14 was not unexpected. Similar to previous NHP studies of AAV delivery of human transgene products, the decline in transgene product expression correlated with a humoral immune response to the foreign human transgene product (anti-human GLA antibodies) (FIG. 41).

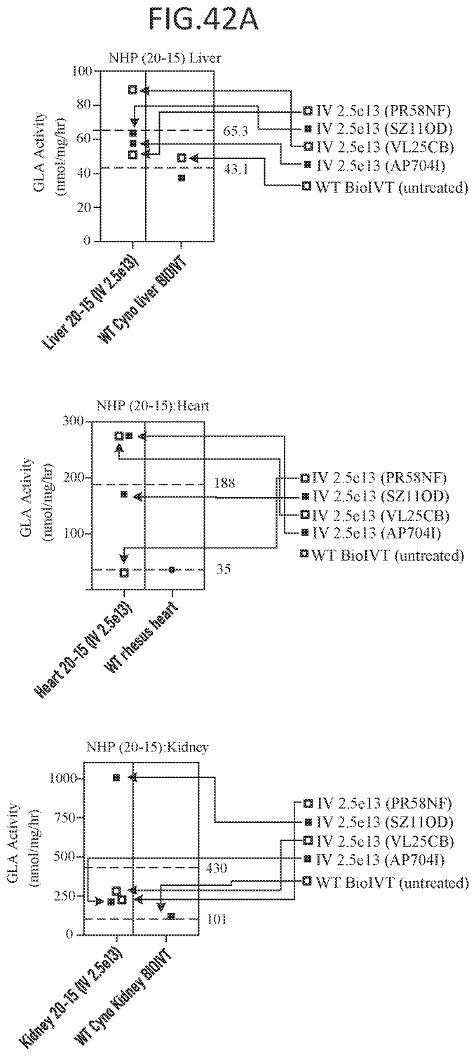

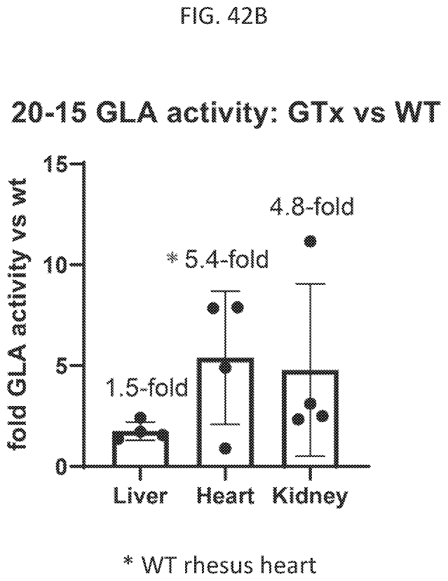

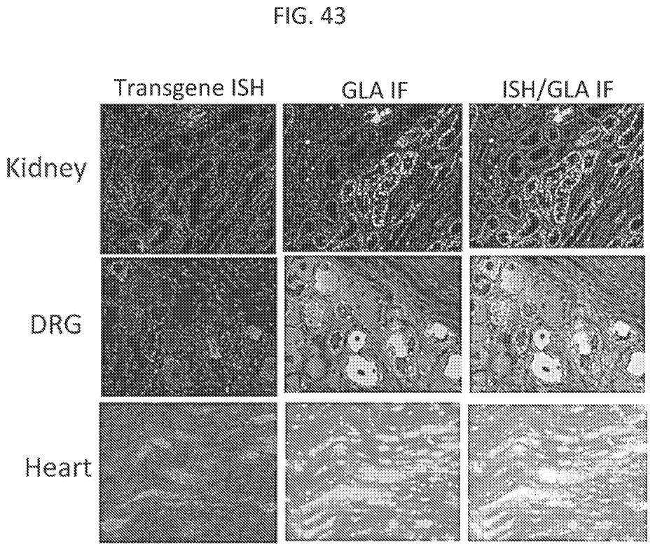

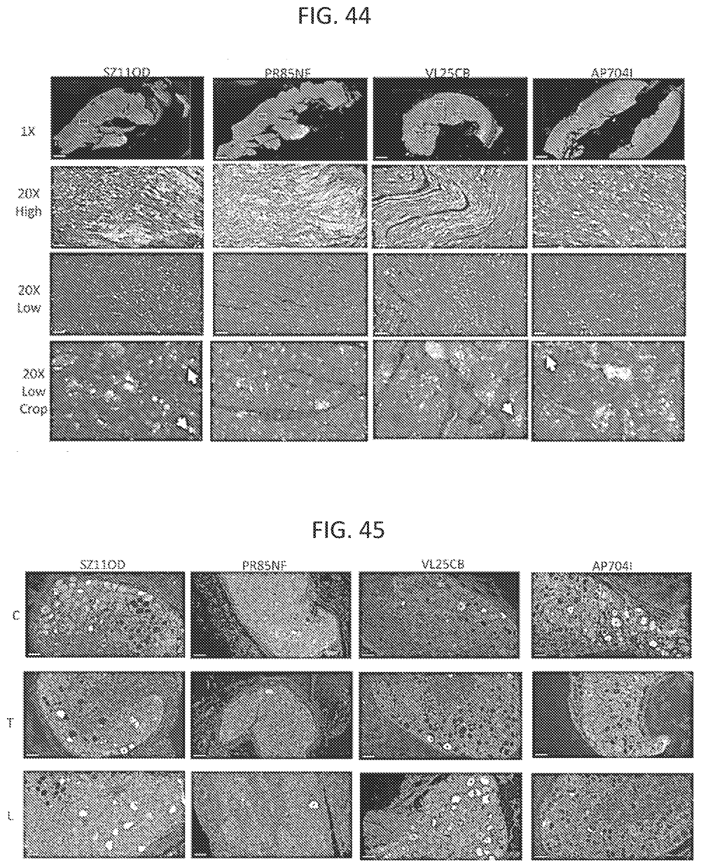

Intravenous administration of AAVhu68.hGLAco(D233C_I359C) also led to significant levels of transgene product expression (GLA enzyme activity) in the heart, liver, and kidney 60 days post treatment (FIG. 42A). The largest fold increases in GLA enzyme activity were observed in the heart and kidney (FIG. 42B). The mean fold-increases in GLA enzyme activity in NHPs at 2.5×10¹³ GC/kg were 4.4 (437%), 0.5 (51%), and 3.3 (325%) in heart, liver, and kidney, respectively. These were of similar magnitude to the increase observed in the KO mouse at doses of 2.5×10¹² GC/kg and 5.0×10¹² GC/kg (FIG. 27-FIG. 29), which demonstrated robust GL-3 clearance (FIG. 31A and FIG. 31B, FIG. 32A, FIG. 32B). FIG. 43-FIG. 45 show transgene expression (ISH) and transgene product (IHC) in heart, kidney, and DRG.

Cumulatively, the study confirms that administration of AAVhu68.hGLAco(D233C_I359C) was well-tolerated in NHPs at a dose of 2.5×10¹³ GC/kg, and resulted in a significant increase in transgene product expression (GLA enzyme activity) in target tissues for the treatment of Fabry disease (kidney, heart, DRG).

### Example 7: High Dose Pharmacology Study to Evaluate AAVhu68.hGLAco (D233C_I359C) Administered Intravenously in Cynomolgus Macaques

Adult NHPs (N=3) received a single IV dose of AAVhu68.hGLAco(D233C_I359C) at a dose of 5.0×10¹³ GC/kg. In-life evaluations include clinical observations performed daily, body weights, clinical pathology of the blood (CBC, coagulation panel, serum chemistry [including the cardiac biomarker troponin I]), and cardiac function assessment (ECG and echocardiography). On Day 60±3 post vector administration, all animals are necropsied. At necropsy, tissues are collected for histopathological examination. Target tissues are collected for vector biodistribution analysis and comprehensive histological evaluation of transgene product expression localization (human GLA ISH [mRNA] and human GLA IHC [protein]). PBMC, splenocytes, and liver lymphocytes are also collected to measure T cell responses to the capsid and transgene product (IFN-γ ELlspot).

| | Study Day | | | | | | |
|---|---|---|---|---|---|---|---|
| | | Day | Day | Day | Day | Day | Day |
| Parameter | BL^{a} | 0 | 3 | 7 ± 1 | 14 ± 1 | 28 ± 2 | 60 ± 3 |
| Dosing - IV administration of | | X | | | | | |
| AAVhu68.CB7.CI.hGLAco | | | | | | | |
| (D233C_I359C).WPRE.rBG (n = 4) | | | | | | | |
| Clinical observations | Daily | | | | | | |
| Body weight, temperature, respiratory rate, | X | X | X | X | X | X | X |
| heart rate | | | | | | | |
| EKG and electrocardiogramhy | X | | | | | | X |
| Biomarker (plasma) - Transgene product | | X | X | X | X | X | X |
| expression, | | | | | | | |
| Anti-transgene product antibodies (ADAs) | | | | | | | |
| Clinical Pathology - Blood (CBC, serum | X | X | X | X | X | X | X |
| clinical chemistry, coagulation)^{b} | | | | | | | |
| Immunology - NAbs against capsid^{c} | X | | | | | | X |
| Immunology - T-cell response to capsid or | X | | | | | | X |
| transgene product (PBMCs) | | | | | | | |
| Necropsy and sample collection^{d} | | | | | | | X |

Abbreviations: ADA = anti-drug antibodies; BL = baseline; ELISpot = enzyme-linked immunosorbent spot; GLA = α-galactosidase A; IFN-γ = interferon gamma; IHC = immunohistochemistry; IN = intranasal; ISH = in situ hybridization; mRNA = messenger ribonucleic acid; MS = mass spectrometry; Nabs = neutralizing antibodies; PBMCs = peripheral blood mononuclear cells.
^{a}BL was up to 21 days prior to dosing.
^{b}Serum chemistry analysis included troponin I (cardiac biomarker).
^{c}Samples were collected and stored for future analysis.
^{d}Tissues were collected for histopathology, vector biodistribution analysis, and histological evaluation of transgene product expression (human GLA ISH staining [mRNA] and human GLA IHC staining [protein]). Liver and spleen lymphocytes were collected for measurement of T-cell responses to the capsid or transgene product (IFN-γ ELISpot).

### Example 8: Efficacy of AAVhu68.hGLAco(D233C_I359C) Following IV Administration to Fabry Mice to Determine the MED

This pharmacology study aims to determine the MED and evaluate the pharmacology and histopathology (efficacy and safety) of IV administration of AAVhu68.hGLAco(D233C_I359C) in the aggravated Fabry mouse model (Gla KO/TgG3S). The study includes N=144 animals and two necropsy time points. Four dose levels of vector are evaluated. The dose levels are selected based on pilot efficacy data in mice and NHP.

As summarized in the table below, adult (2- to 3-month old) male aggravated Fabry mice (Gla KO/TgG3S) have been administered either AAVhu68.hGLAco(D233C_I359C) at one of four dose levels to be determined (5.0×10¹² GC/kg, 1.0×10¹³ GC/kg, 2.5×10¹³ GC/kg, or 5.0×10¹³ GC/kg) or vehicle (PBS). Normal male WT and WT/TgG3S males have been administered vehicle as a control. Female aggravated Gla KO/TgG3S mice have also received either AAVhu68.hGLAco(D233C_I359C) at the highest dose (5.0×10¹³ GC/kg) or vehicle. Sixteen animals have been enrolled in each group. Half of the animals (8 per group) will be sacrificed on Study Day 120, and the other half (8 per group) will be necropsied when at least 80% of the vehicle-treated Gla KO/TgG3S mice have reached a humane endpoint (defined as severe tremors and ataxia causing ambulation impairment and/or body weight loss ≥20% of peak body weight).

| | Group 1 | Group 2 | Group 3 | Group 4 | Group 5 | Group 6 | Group 7 | Group 8 | Group 9 |
|---|---|---|---|---|---|---|---|---|---|
| Number | 16 | 16 | 16 | 16 | 16 | 16 | 16 | 16 | 16 |
| of Mice | | | | | | | | | |
| Sex | M | M | M | M | F | M | M | M | F |
| Genotype | Gla KO/ | Gla KO/ | Gla KO/ | Gla KO/ | Gla KO/ | WT | WT/ | Gla KO/ | Gla KO/ |
| | TgG3S | TgG3S | TgG3S | TgG3S | TgG3S | | TgG3S | TgG3S | TgG3S |
| Test Article | Vector | Vector | Vector | Vector | Vector | Vehicle | Vehicle | Vehicle | Vehicle |
| ROA | IV | IV | IV | IV | IV | IV | IV | IV | IV |
| Vector Dose | 5.0 × 10¹² | 1.0 × 10¹³ | 2.5 × 10¹³ | 5.0 × 10¹³ | 5.0 × 10¹³ | N/A | N/A | N/A | N/A |
| | GC/kg | GC/kg | GC/kg | GC/kg | GC/kg | | | | |

| | |
|---|---|
| Necropsy | Necropsy Timepoint #1: Day 120 ± 5 days (n = 8/group) |
| | Necropsy Timepoint #2: When ≥80% of both Groups |
| | 8 and 9 reach humane euthanasia (n = 8/group) |

Abbreviations: F = female; Gla = α-galactosidase A (gene); IV = intravenous; KO = knockout; M = male; N/A = not applicable; ROA = route of administration; TBD = to be determined; WT = wild type.

In-life assessments include viability checks performed daily to monit for survival, body weight measurements, clinical observations, thermosensory function assessment (hotplate latency), serum blood urea nitrogen (BUN) levels, urine osmolality, urine volume, and evaluation of serum transgene expression (GLA enzyme activity). Necropsies are performed at the humane endpoint and an earlier time point (Day 120). At necropsy, a comprehensive list of tissues are collected for histopathological evaluation. Additional tissues (DRG, heart, kidney) are collected to assess GL-3 storage (GL-3 IHC). Target tissues are also collected for a transgene expression assay (GLA enzyme activity) and GL-3 quantification by LC-MS/MS (brain, heart, kidney, liver, large intestine). Blood is collected for CBC/differentials and serum clinical chemistry analysis, including BUN levels. Plasma is also collected to evaluate transgene product expression (GLA enzyme activity) and lyso-Gb3 storage.

Personnel performing the in-life evaluations for body weight and the hotplate assay are blinded to the treatment condition and genotype of each mouse.

The MED is determined based upon analysis of survival benefit, body weight, thermosensory function (assessed using the hotplate assay), renal function (assessed by BUN levels, urine volume, and urine osmolality), correction of GL-3 lysosomal storage in target tissues, and transgene product expression (GAL activity levels) in disease-relevant target organs.

### Example 9: Toxicology Study of Intravenous Administration of AAVhu68.hGLAco(D233C_I359C) to Adult Non-Human Primates

A 180-day GLP-compliant toxicology study is performed to assess the safety, tolerability, transgene product expression, biodistribution, and excretion profile of AAVhu68.hGLAco(D233C_I359C) following a single IV administration to cynomolgus macaque NHPs at a low dose (1.0×10¹³ GC/kg), mid-dose (2.5×10¹³ GC/kg), or high dose (5.0×10¹³ GC/kg). Additional NHPs are administered vehicle (PBS) as a control.

The NHP (cynomolgus macaque) was selected for the planned toxicology study. This model was selected because we have substantial experience with the application of AAV vectors in NHPs, and the toxicological and immune responses of the NHP closely represent that of a human. Adult NHPs (2 to 8 years old) were selected to be representative of the patient population for the planned clinical trial. Males and females will be included in the study.

The IV route was selected because systemic administration provides the best transduction and transgene product expression in the disease-relevant target tissues (DRG, kidney, and heart) and non-disease relevant (liver) target tissue.

TABLE
Groups for NHP toxicology study

| | Group 1 | Group 2 | Group 2 | Group 3 |
|---|---|---|---|---|
| Parameter | (Control) | (Low Dose) | (Mid-Dose) | (High Dose) |
| Number of | 2 | 3 | 3 | 3 |
| NHPs | | | | |
| Sex | M + F | M + F | M + F | M + F |
| Age | Adult (2 to | Adult (2 to | Adult (2 to | Adult (2 to |
| | 8 years) | 8 years) | 8 years) | 8 years) |
| Test Article | Vehicle | Vector | Vector | Vector |
| | (PBS) | | | |
| Route of | IV | IV | IV | IV |
| Administration | | | | |
| Vector Dose | N/A | 1.0 × 10¹³ | 2.5 × 10¹³ | 5.0 × 10¹³ |
| | | GC/kg | GC/kg | GC/kg |
| Necropsy Day | Day 180 | Day 180 | Day 180 | Day 180 |

Cage-side clinical observations and evaluation of vital signs, body weights, and clinical pathology of the blood (CBC with differentials, clinical chemistries, coagulation panel) and CSF (cytology and chemistry) are obtained at frequent intervals throughout the study. Complete blood count (CBC), liver parameters, and complement activation are monitored because acute liver toxicity, thrombocytopenia, and complement activation are known toxicities after systemic AAV administration.

A troponin I test is included as part of the clinical pathology panel, along with echocardiogram assessments at baseline and every 30 days after treatment to monitor for signs of cardiotoxicity because AAVhu68 demonstrates a high tropism for cardiac tissues after IV administration.

Neurologic examinations are performed at baseline, on Day 14, Day 28, and then every 30 days thereafter. Sensory nerve conduction studies (NCS) of the bilateral median nerves are performed at baseline, Day 28, Day 60, and Day 180 to monitor for signs of DRG sensory neuron degeneration. These time points were selected based on the known kinetics of sensory neuron degeneration in NHPs, which appears 14-21 days after vector administration and are detectable on median nerve NCS by Day 30. For the neurologic examination, assessments are divided into five sections evaluating mentation, posture and gait, proprioception, cranial nerves, and spinal reflexes. The tests for each assessment ate performed in the same order each time. Assessors for the neurologic examination are not formally blinded to the treatment group; however, assessors typically remain unaware of treatment group at the time of assessment. Numerical scores are given for each assessment category as applicable and recorded (normal: 1; abnormal: 2; decreased: 3; increased: 4; none: 5; N/A: not applicable). For sensory NCS, the Nicolet EDX® system (Natus Neurology) and Viking® analysis software is used to measure sensory nerve action potential amplitudes and conduction velocities. The assessors performing the NCS analysis are formally blinded to treatment group.

The expression of the transgene product (GLA enzyme activity) is measured in serum. Samples are collected at frequent intervals during the expected onset, peak, and plateau of transgene expression). Anti-transgene product antibodies (ie, anti-drug antibodies [ADAs]) are likewise evaluated at corresponding time points in serum using an enzyme-linked immunosorbent assay (ELISA) to assess potential antibody responses to the foreign human transgene product that may occur systemically.

Neutralizing antibody responses against the AAVhu68 capsid are measured at baseline to assess the impact on vector transduction (biodistribution) and then monthly thereafter to assess the kinetics of the NAb response. Peripheral blood mononuclear cells are collected to evaluate T-cell responses to the capsid and/or transgene product using an IFN-γ ELISpot assay. The time points for PBMC collection were selected because T-cell and B-cell immune responses typically occur within 30 days in NHPs. At necropsy, tissue-resident lymphocytes from the spleen and liver are also collected for evaluation of T-cell responses to the capsid and/or transgene product.

Serum and CSF is collected to assess vector distribution, and urine and feces are collected to assess vector excretion (shedding). These samples are collected at frequent time points and quantified by quantitative polymerase chain reaction (qPCR) to enable assessment of the kinetics of vector distribution and excretion post treatment. Samples of CSF and serum are also collected and archived for future possible analysis in case any finding warrants analysis.

At necropsy on Day 180, a comprehensive list of tissues are collected for histopathology and vector biodistribution analysis. Tissues are also collected to assess transgene product expression. All tissues are selected to include possible target tissues of Fabry disease (kidney, heart, DRG, intestine) and/or highly perfused peripheral organs (such as the liver and kidneys). In addition, lymphocytes are harvested from the liver, spleen, and bone marrow to evaluate the presence of T-cells reactive to the capsid and/or transgene product in these organs at the time of necropsy.

### Sequence Listing Free Text

The following information is provided for sequences containing free text under numeric identifier <223>.

| SEQ ID NO: | Free Text under <223> |
|---|---|
| 3 | <223> synthetic construct |
| 4 | <223> synthetic construct |
| 5 | <223> synthetic construct |
| 6 | <223> CB7.CI.hGLAco(D233C/I359C).WPRE.rBG |
| | <220> |
| | <221> repeat_region |
| | <222> (1) . . . (130) |
| | <223> 5′ ITR |
| | <220> |
| | <221> misc_feature |
| | <222> (198) . . . (579) |
| | <223> CMV immediate early enhancer |
| | <220> |
| | <221> promoter |
| | <222> (582) . . . (863) |
| | <223> CB promoter |
| | <220> |
| | <221> Intron |
| | <222> (956) . . . (1928) |
| | <223> chicken beta-actin intron |
| | <220> |
| | <221> CDS |
| | <222> (1948) . . . (3240) |
| | <223> hGLAco.D233C.I359C |
| | <220> |
| | <221> misc_feature |
| | <222> (2353) . . . (2372) |
| | <223> P18 |
| | <220> |
| | <221> misc_feature |
| | <222> (2644) . . . (2646) |
| | <223> D233C |
| | <220> |
| | <221> misc_feature |
| | <222> (3022) . . . (3024) |
| | <223> I359C |
| | <220> |
| | <221> misc_feature |
| | <222> (3253) . . . (3841) |
| | <223> WPRE |
| | <220> |
| | <221> misc_feature |
| | <222> (3609) . . . (3628) |
| | <223> P19 |
| | <220> |
| | <221> polyA_signal |
| | <222> (3953) . . . (4079) |
| | <223> Rabbit globin poly A |
| | <220> |
| | <221> repeat_region |
| | <222> (4168) . . . (4297) |
| | <223> 3′ ITR |
| 7 | <223> synthetic construct |
| 8 | <223> TBG.PI.hGLAnat.WPRE.bGH |
| | <220> |
| | <221> repeat_region |
| | <222> (1) . . . (168) |
| | <223> 5′ ITR |
| | <220> |
| | <221> enhancer |
| | <222> (211) . . . (310) |
| | <223> alpha mic/bik |
| | <220> |
| | <221> enhancer |
| | <222> (317) . . . (416) |
| | <223> alpha mic/bik |
| | <220> |
| | <221> misc_feature |
| | <222> (431) . . . (907) |
| | <223> TBG promoter |
| | <220> |
| | <221> Intron |
| | <222> (939) . . . (1071) |
| | <223> SV40 misc intron |
| | <220> |
| | <221> CDS |
| | <222> (1100) . . . (2392) |
| | <223> hGLA natural |
| | <220> |
| | <221> misc_feature |
| | <222> (2411) . . . (2952) |
| | <223> WPRE |
| | <220> |
| | <221> polyA_signal |
| | <222> (2959) . . . (3173) |
| | <223> BGH pA |
| | <220> |
| | <221> repeat_region |
| | <222> (3223) . . . (3390) |
| | <223> 3′ ITR |
| 9 | <223> synthetic construct |
| 10 | <223> CB7.CI.hGLAnat.WPRE.RBG |
| | <220> |
| | <221> repeat_region |
| | <222> (1) . . . (130) |
| | <223> 5′ ITR |
| | <220> |
| | <221> misc_feature |
| | <222> (198) . . . (579) |
| | <223> CMV IE promoter |
| | <220> |
| | <221> promoter |
| | <222> (582) . . . (863) |
| | <223> CB promoter |
| | <220> |
| | <221> TATA_signal |
| | <222> (836) . . . (839) |
| | <223> TATA |
| | <220> |
| | <221> Intron |
| | <222> (956) . . . (1928) |
| | <223> chicken beta-actin intron |
| | <220> |
| | <221> CDS |
| | <222> (1950) . . . (3242) |
| | <223> hGLA natural |
| | <220> |
| | <221> misc_feature |
| | <222> (3317) . . . (3905) |
| | <223> WPRE |
| | <220> |
| | <221> poly A_signal |
| | <222> (3950) . . . (4076) |
| | <223> Rabbit globin poly A |
| | <220> |
| | <221> repeat_region |
| | <222> (4165) . . . (4294) |
| | <223> 3′ ITR |
| 11 | <223> synthetic construct |
| 12 | <223> TBG.PI.hGLAco.WPRE.bGH |
| | <220> |
| | <221> repeat_region |
| | <222> (1) . . . (168) |
| | <223> 5′ ITR |
| | <220> |
| | <221> enhancer |
| | <222> (211) . . . (310) |
| | <220> |
| | <221> enhancer |
| | <222> (317) . . . (416) |
| | <220> |
| | <221> Intron |
| | <222> (939) . . . (1071) |
| | <223> SV40 misc intron |
| | <220> |
| | <221> CDS |
| | <222> (1100) . . . (2392) |
| | <223> hGLAco |
| | <220> |
| | <221> misc_feature |
| | <222> (2411) . . . (2952) |
| | <223> WPRE |
| | <220> |
| | <221> polyA_signal |
| | <222> (2959) . . . (3173) |
| | <223> BGH pA |
| | <220> |
| | <221> repeat_region |
| | <222> (3223) . . . (3390) |
| | <223> 3′ ITR |
| 13 | <223> synthetic construct |
| 14 | <223> CB7.CI.hGLAco.WPRE.RBG |
| | <220> |
| | <221> repeat_region |
| | <222> (1) . . . (130) |
| | <223> 5′ ITR |
| | <220> |
| | <221> repeat_region |
| | <222> (198) . . . (579) |
| | <223> CMV IE promoter |
| | <220> |
| | <221> promoter |
| | <222> (582) . . . (863) |
| | <223> CB promoter |
| | <220> |
| | <221> TATA_signal |
| | <222> (582) . . . (863) |
| | <223> TATA |
| | <220> |
| | <221> Intron |
| | <222> (956) . . . (1928) |
| | <223> chicken beta-actin intron |
| | <220> |
| | <221> CDS |
| | <222> (1950) . . . (3242) |
| | <223> hGLAco |
| | <220> |
| | <221> misc_feature |
| | <222> (3317) . . . (3905) |
| | <223> WPRE |
| | <220> |
| | <221> polyA_signal |
| | <222> (3950) . . . (4076) |
| | <223> Rabbit globin poly A |
| | <220> |
| | <221> repeat_region |
| | <222> (4165) . . . (4294) |
| | <223> 3′ ITR |
| 15 | <223> synthetic construct |
| 16 | <223> TBG.PI.hGLAco(M51C_G360C).WPRE.bGH |
| | <220> |
| | <221> repeat_region |
| | <222> (1) . . . (168) |
| | <223> 5′ ITR |
| | <220> |
| | <221> enhancer |
| | <222> (211) . . . (310) |
| | <223> alpha mic/bik |
| | <220> |
| | <221> enhancer |
| | <222> (317) . . . (416) |
| | <223> alpha mic/bik |
| | <220> |
| | <221> misc_feature |
| | <222> (431) . . . (907) |
| | <223> TBG promoter |
| | <220> |
| | <221> Intron |
| | <222> (939) . . . (1071) |
| | <223> SV40 misc intron |
| | <220> |
| | <221> CDS |
| | <222> (1094) . . . (2386) |
| | <223> hGLAco.M51C.G360C |
| | <220> |
| | <221> misc_feature |
| | <222> (2399) . . . (2940) |
| | <223> WPRE |
| | <220> |
| | <221> polyA_signal |
| | <222> (2947) . . . (3161) |
| | <223> BGH pA |
| | <220> |
| | <221> repeat_region |
| | <222> (3211) . . . (3378) |
| | <223> 3′ ITR |
| 17 | <223> synthetic construct |
| 18 | <223> CB7.CI.hGLAco(M51C_G360C).WPRE.RBG |
| | <220> |
| | <221> repeat_region |
| | <222> (1) . . . (130) |
| | <223> 5′ ITR |
| | <220> |
| | <221> misc_feature |
| | <222> (198) . . . (579) |
| | <223> CMV IE promoter |
| | <220> |
| | <221> promoter |
| | <222> (582) . . . (863) |
| | <223> CB promoter |
| | <220> |
| | <221> TATA_signal |
| | <222> (836) . . . (839) |
| | <223> TATA |
| | <220> |
| | <221> Intron |
| | <222> (956) . . . (1928) |
| | <223> chicken beta-actin intron |
| | <220> |
| | <221> CDS |
| | <222> (1958) . . . (3250) |
| | <223> hGLAco.M51C.G360C |
| | <220> |
| | <221> misc_feature |
| | <222> (2363) . . . (2382) |
| | <223> P18 |
| | <220> |
| | <221> misc_feature |
| | <222> (3263) . . . (3851) |
| | <223> WPRE |
| | <220> |
| | <221> misc_feature |
| | <222> (3619) . . . (3638) |
| | <223> P19 |
| | <220> |
| | <221> polyA_signal |
| | <222> (3896) . . . (4022) |
| | <223> Rabbit globin poly A |
| | <220> |
| | <221> repeat_region |
| | <222> (4111) . . . (4240) |
| | <223> 3′ ITR |
| 22 | <223> AAV9 VP1 Capsid |
| 23 | <223> synthetic construct |
| 26 | <223> synthetic construct |
| 27 | <223> synthetic construct |
| 28 | <223> synthetic construct |
| 29 | <223> synthetic construct |
| 30 | <223> synthetic construct |
| 31 | <223> miR target sequence |
| 32 | <223> miR target sequence |

All patent and non-patent publications cited in this specification are incorporated herein by reference in their entireties. International Patent Application No. PCT/US2019/05567, filed Oct. 10, 2019, U.S. Provisional Patent Application No. 63/089,850, filed Oct. 9, 2020, U.S. Provisional Patent Application No. 63/146,286, filed Feb. 5, 2021, U.S. Provisional Patent Application No. 63/186,092, filed May 8, 2021 and are incorporated by reference herein in their entireties. The sequence listing filed herewith named “19-8855PCT_ST25.txt” and the sequences and text therein are incorporated herein by reference. While the invention has been described with reference to particular embodiments, it will be appreciated that modifications can be made without departing from the spirit of the invention. Such modifications are intended to fall within the scope of the appended claims.

1. A recombinant AAV (rAAV) comprising an AAVhu68 capsid having packaged therein a vector genome, wherein the vector genome comprises a coding sequence for a functional human alpha-galactosidase A (hGLA) and regulatory sequences which direct expression of the hGLA in a target cell,
wherein the coding sequence comprises nucleotides 94 to 1287 of SEQ ID NO: 4, or a sequence at least 85% identical thereto, and wherein the hGLA has a cysteine residue at position 233 and/or position 359 based on the amino acid residue numbering of SEQ ID NO: 2.

2. The rAAV according to claim 1, wherein the hGLA comprises at least amino acids 32 to 429 of SEQ ID NO: 2, or a sequence at least 95% identical thereto.

3. The rAAV according to claim 1, wherein the hGLA comprises amino acids 32 to 429 of SEQ ID NO: 7.

4. The rAAV according to claim 1, wherein the hGLA comprises the native signal peptide.

5. The rAAV according to claim 1, wherein the hGLA comprises a heterologous signal peptide.

6. The rAAV according to claim 1, wherein the hGLA comprises the full length (amino acids 1 to 429) of SEQ ID NO: 17, or a sequence at least 95% identical thereto.

7. The rAAV according to claim 1, wherein the vector genome comprises a tissue-specific promoter.

8. The rAAV according to claim 1, wherein the regulatory sequences comprise a chicken beta-actin promoter with cytomegalovirus enhancer elements, an intron, and a polyA.

9. The rAAV according to claim 1, wherein the regulatory sequences comprise a woodchuck hepatitis virus post-transcriptional regulatory element (WPRE)

10. The rAAV according to claim 1, wherein the vector genome further comprises one or more miRNA target sequences.

11. The rAAV according to claim 1, wherein the vector genome comprises a sequence at least 85% identical to SEQ ID NO: 6.

12. An expression cassette comprising a nucleic acid sequence encoding a functional human alpha-galactosidase A (hGLA) and one or more regulatory sequences which direct expression of the hGLA in a target cell containing the expression cassette,
wherein the nucleic acid sequence comprises nucleotides 94 to 1287 of SEQ ID NO: 4, or a sequence at least 85% identical thereto, and wherein the hGLA has a cysteine residue at position 233 and/or position 359 based on the amino acid residue numbering of SEQ ID NO: 2 or SEQ ID NO: 7.

13. The expression cassette according to claim 12, wherein the hGLA comprises amino acids 32 to 429 of SEQ ID NO: 7.

14. The expression cassette according to claim 1213, wherein the hGLA comprises the native signal peptide.

15. The expression cassette according to claim 12, wherein the hGLA comprises a heterologous signal peptide.

16. The expression cassette according to claim 12, wherein the hGLA comprises the full length (amino acids 1 to 429) of SEQ ID NO: 7, or a sequence at least 95% identical thereto.

24.-24. (canceled)

25. A plasmid comprising the expression cassette according to claim 12.

26. A host cell comprising the plasmid according to claim 25.

27. A pharmaceutical composition comprising the rAAV according to claim 1.

28. A method of treating a human subject diagnosed with GLA-deficiency (Fabry disease), the method comprising administering to the subject the pharmaceutical composition according to claim 27.

30.-30. (canceled)


SEQUENCE LISTING (32 sequences)

SEQ ID NO: 1 - DNA, 1287 bp, Homo sapiens
  CDS (1)..(1287)
atgcagctgaggaacccagaactacatctgggctgcgcgcttgcgcttcgcttcctggccctcgtttcctgggacatccctggggctagagcactggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagtagatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactt

SEQ ID NO: 2 - protein, 429 aa, Homo sapiens
MQLRNPELHLGCALALRFLALVSWDIPGARALDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 3 - DNA, 1287 bp, Artificial Sequence
  synthetic construct
atgcagctgagaaatcccgagctgcacctgggctgtgccctggctctgagatttctggccctggtgtcttgggacatccctggcgctagagccctggataacggcctggccagaacacctacaatgggctggctgcactgggagagattcatgtgcaacctggactgccaagaggaacccgacagctgcatcagcgagaagctgttcatggaaatggccgagctgatggtgtccgaaggctggaaggacgccggctacgagtacctgtgcatcgacgactgttggatggcccctcagagagactctgagggcagactgcaggccgatcctcagagatttccccacggcattagacagctggccaactacgtgcacagcaagggcctgaagctgggcatctacgccgacgtgggcaacaagacctgtgccggctttcctggcagcttcggctactacgatatcgacgcccagaccttcgccgattggggagtcgatctgctgaagttcgacggctgctactgcgacagcctggaaaatctggccgacggctacaagcacatgtcactggccctgaatcggaccggccgcagcatcgtgtactcttgcgagtggcccctgtatatgtggcccttccagaagcctaactacaccgagatcagacagtactgcaaccactggcggaacttcgccgacatcgacgatagctggaagtccatcaagagcatcctggactggaccagcttcaatcaagagcggatcgtggacgtggcaggacctggcggatggaacgatcctgacatgctggtcatcggcaacttcggcctgagctggaaccagcaagtgacccagatggccctgtgggccattatggccgctcctctgttcatgagcaacgacctgagacacatcagccctcaggccaaggctctgctgcaggacaaggatgtgatcgctatcaaccaggatcctctgggcaagcagggctaccagctgagacagggcgacaatttcgaagtgtgggaaagacccctgagcggactggcttgggccgtcgccatgatcaacagacaagagatcggcggaccccggtcctacacaattgccgtggcttctctcggcaaaggcgtggcctgtaatcccgcctgctttatcacacagctgctgcccgtgaagagaaagctgggcttttacgagtggaccagcagactgcggagccacatcaatcctaccggcacagtgctgctgcagctggaaaacacaatgcagatgagcctgaaggacctgctg

SEQ ID NO: 4 - DNA, 1287 bp, Artificial Sequence
  synthetic construct
atgcaactgagaaatcctgaactgcacctgggctgcgccctggctctgagatttctggctctggtgtcctgggacatccctggcgctagagccctggataacggcctggccagaacacctacaatgggctggctgcactgggagagattcatgtgcaacctggactgccaagaggaacccgacagctgcatcagcgagaagctgttcatggaaatggccgagctgatggtgtccgaaggctggaaggacgccggctacgagtacctgtgcatcgacgactgttggatggcccctcagagagactctgagggcagactgcaggccgatcctcagagatttccccacggcattagacagctggccaactacgtgcacagcaagggcctgaagctgggcatctacgccgacgtgggcaacaagacctgtgccggctttcctggcagcttcggctactacgatatcgacgcccagaccttcgccgattggggagtcgatctgctgaagttcgacggctgctactgcgacagcctggaaaatctggccgacggctacaagcacatgtctctggccctgaatcggaccggcagatccatcgtgtacagctgcgagtggcccctgtacatgtggcccttccagaagcctaactacaccgagatcagacagtactgcaaccactggcggaacttcgccgacatctgcgatagctggaagtccatcaagagcatcctggactggaccagcttcaatcaagagcggatcgtggacgtggcaggacctggcggatggaacgatcctgacatgctggtcatcggcaacttcggcctgagctggaaccagcaagtgacccagatggccctgtgggccattatggccgctcctctgttcatgagcaacgacctgagacacatcagccctcaggccaaggctctgctgcaggacaaggatgtgatcgctatcaaccaggatcctctgggcaagcagggctaccagctgagacagggcgacaatttcgaagtgtgggaaagacccctgagcggactggcttgggccgtcgccatgatcaaccggcaagagtgcggcggccccagatcctacacaatcgccgtggccagtctcggcaaaggcgtggcatgtaatcccgcctgcttcatcacacagctgctgcccgtgaagagaaagctgggcttttacgagtggaccagcagactgcggagccacatcaatcctaccggcacagtgctgctgcagctggaaaacaccatgcagatgagcctgaaggacctgctg

SEQ ID NO: 5 - DNA, 1287 bp, Artificial Sequence
  synthetic construct
atgcaactgagaaatcctgaactgcacctgggctgcgccctggctctgagatttctggctctggtgtcctgggacatccctggcgctagagccctggataacggcctggccagaacacctacaatgggctggctgcactgggagagattctgctgcaacctggactgccaagaggaacccgacagctgcatcagcgagaagctgttcatggaaatggccgagctgatggtgtccgaaggctggaaggacgccggctacgagtacctgtgcatcgacgactgttggatggcccctcagagagactctgagggcagactgcaggccgatcctcagagatttccccacggcattagacagctggccaactacgtgcacagcaagggcctgaagctgggcatctacgccgacgtgggcaacaagacctgtgccggctttcctggcagcttcggctactacgatatcgacgcccagaccttcgccgattggggagtcgatctgctgaagttcgacggctgctactgcgacagcctggaaaatctggccgacggctacaagcacatgtctctggccctgaatcggaccggcagatccatcgtgtacagctgcgagtggcccctgtacatgtggcccttccagaagcctaactacaccgagatcagacagtactgcaaccactggcggaacttcgccgacatcgacgatagctggaagtccatcaagagcatcctggactggaccagcttcaatcaagagcggatcgtggacgtggcaggacctggcggatggaacgatcctgacatgctggtcatcggcaacttcggcctgagctggaaccagcaagtgacccagatggccctgtgggccattatggccgctcctctgttcatgagcaacgacctgagacacatcagccctcaggccaaggctctgctgcaggacaaggatgtgatcgctatcaaccaggatcctctgggcaagcagggctaccagctgagacagggcgacaatttcgaagtgtgggaaagacccctgagcggactggcttgggccgtcgccatgatcaaccggcaagagatttgcggccccagatcctacacaatcgccgtggccagtctcggcaaaggcgtggcatgtaatcccgcctgcttcatcacacagctgctgcccgtgaagagaaagctgggcttttacgagtggaccagcagactgcggagccacatcaatcctaccggcacagtgctgctgcagctggaaaacaccatgcagatgagcctgaaggacctgctg

SEQ ID NO: 6 - DNA, 4297 bp, Artificial Sequence
  CB7.CI.hGLAco(D233C/I359C).WPRE.rBG
  repeat_region (1)..(130): 5′ ITR
  misc_feature (198)..(579): CMV immediate early enhancer
  promoter (582)..(863): CB promoter
  Intron (956)..(1928): chicken beta-actin intron
  CDS (1948)..(3240): hGLAco.D233C.I359C
  misc_feature (2353)..(2372): P18
  misc_feature (2644)..(2646): D233C
  misc_feature (3022)..(3024): I359C
  misc_feature (3253)..(3841): WPRE
  misc_feature (3609)..(3628): P19
  polyA_signal (3953)..(4079): Rabbit globin poly A
  repeat_region (4168)..(4297): 3′ ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgcggccgcgccaccatgcaactgagaaatcctgaactgcacctgggctgcgccctggctctgagatttctggctctggtgtcctgggacatccctggcgctagagccctggataacggcctggccagaacacctacaatgggctggctgcactgggagagattcatgtgcaacctggactgccaagaggaacccgacagctgcatcagcgagaagctgttcatggaaatggccgagctgatggtgtccgaaggctggaaggacgccggctacgagtacctgtgcatcgacgactgttggatggcccctcagagagactctgagggcagactgcaggccgatcctcagagatttccccacggcattagacagctggccaactacgtgcacagcaagggcctgaagctgggcatctacgccgacgtgggcaacaagacctgtgccggctttcctggcagcttcggctactacgatatcgacgcccagaccttcgccgattggggagtcgatctgctgaagttcgacggctgctactgcgacagcctggaaaatctggccgacggctacaagcacatgtctctggccctgaatcggaccggcagatccatcgtgtacagctgcgagtggcccctgtacatgtggcccttccagaagcctaactacaccgagatcagacagtactgcaaccactggcggaacttcgccgacatctgcgatagctggaagtccatcaagagcatcctggactggaccagcttcaatcaagagcggatcgtggacgtggcaggacctggcggatggaacgatcctgacatgctggtcatcggcaacttcggcctgagctggaaccagcaagtgacccagatggccctgtgggccattatggccgctcctctgttcatgagcaacgacctgagacacatcagccctcaggccaaggctctgctgcaggacaaggatgtgatcgctatcaaccaggatcctctgggcaagcagggctaccagctgagacagggcgacaatttcgaagtgtgggaaagacccctgagcggactggcttgggccgtcgccatgatcaaccggcaagagtgcggcggccccagatcctacacaatcgccgtggccagtctcggcaaaggcgtggcatgtaatcccgcctgcttcatcacacagctgctgcccgtgaagagaaagctgggcttttacgagtggaccagcagactgcggagccacatcaatcctaccggcacagtgctgctgcagctggaaaacaccatgcagatgagcctgaaggacctgctgtgatagaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtctcgaatcaagcggccgcaagcatgctgagctccagatctggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 7 - protein, 429 aa, Artificial Sequence
  Synthetic Construct
MQLRNPELHLGCALALRFLALVSWDIPGARALDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADICDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQECGGPRSYTIAVASLGKGVACNPACFITQLLPVKRKLGFYEWTSRLRSHINPTGTVLLQLENTMQMSLKDLL

SEQ ID NO: 8 - DNA, 3390 bp, Artificial Sequence
  TBG.PI.hGLAnat.WPRE.bGH
  repeat_region (1)..(168): 5′ ITR
  enhancer (211)..(310): alpha mic/bik
  enhancer (317)..(416): alpha mic/bik
  misc_feature (431)..(907): TBG promoter
  Intron (939)..(1071): SV40 misc intron
  CDS (1100)..(2392): hGLA natural
  misc_feature (2411)..(2952): WPRE
  polyA_signal (2959)..(3173): BGH pA
  repeat_region (3223)..(3390): 3′ ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagactgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgcgaattcgccaccatgcagctgaggaacccagaactacatctgggctgcgcgcttgcgcttcgcttcctggccctcgtttcctgggacatccctggggctagagcactggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagtagatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaatgatgtacaaagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 9 - protein, 429 aa, Artificial Sequence; identical to SEQ ID NO: 2
  Synthetic Construct

SEQ ID NO: 10 - DNA, 4294 bp, Artificial Sequence
  CB7.CI.hGLAnat.WPRE.RBG
  repeat_region (1)..(130): 5′ ITR
  misc_feature (198)..(579): CMV IE promoter
  promoter (582)..(863): CB promoter
  TATA_signal (836)..(839): TATA
  Intron (956)..(1928): chicken beta-actin intron
  CDS (1950)..(3242): hGLA natural
  misc_feature (3317)..(3905): WPRE
  polyA_signal (3950)..(4076): Rabbit globin poly A
  repeat_region (4165)..(4294): 3′ ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaatagcttcgaattcgccaccatgcagctgaggaacccagaactacatctgggctgcgcgcttgcgcttcgcttcctggccctcgtttcctgggacatccctggggctagagcactggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagtagatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaatgatgtacaagtaaagatctgcggccgcgtggtacctctagagtcgacccgggcggcctcgaatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 11 - protein, 429 aa, Artificial Sequence; identical to SEQ ID NO: 2
  Synthetic Construct

SEQ ID NO: 12 - DNA, 3390 bp, Artificial Sequence
  TBG.PI.hGLAco.WPRE.bGH
  repeat_region (1)..(168): 5′ ITR
  enhancer (211)..(310)
  enhancer (317)..(416)
  Intron (939)..(1071): SV40 misc intron
  CDS (1100)..(2392): hGLAco
  misc_feature (2411)..(2952): WPRE
  polyA_signal (2959)..(3173): BGH pA
  repeat_region (3223)..(3390): 3′ ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagactgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgcgaattcgccaccatgcagctgagaaatcccgagctgcacctgggctgtgccctggctctgagatttctggccctggtgtcttgggacatccctggcgctagagccctggataacggcctggccagaacacctacaatgggctggctgcactgggagagattcatgtgcaacctggactgccaagaggaacccgacagctgcatcagcgagaagctgttcatggaaatggccgagctgatggtgtccgaaggctggaaggacgccggctacgagtacctgtgcatcgacgactgttggatggcccctcagagagactctgagggcagactgcaggccgatcctcagagatttccccacggcattagacagctggccaactacgtgcacagcaagggcctgaagctgggcatctacgccgacgtgggcaacaagacctgtgccggctttcctggcagcttcggctactacgatatcgacgcccagaccttcgccgattggggagtcgatctgctgaagttcgacggctgctactgcgacagcctggaaaatctggccgacggctacaagcacatgtcactggccctgaatcggaccggccgcagcatcgtgtactcttgcgagtggcccctgtatatgtggcccttccagaagcctaactacaccgagatcagacagtactgcaaccactggcggaacttcgccgacatcgacgatagctggaagtccatcaagagcatcctggactggaccagcttcaatcaagagcggatcgtggacgtggcaggacctggcggatggaacgatcctgacatgctggtcatcggcaacttcggcctgagctggaaccagcaagtgacccagatggccctgtgggccattatggccgctcctctgttcatgagcaacgacctgagacacatcagccctcaggccaaggctctgctgcaggacaaggatgtgatcgctatcaaccaggatcctctgggcaagcagggctaccagctgagacagggcgacaatttcgaagtgtgggaaagacccctgagcggactggcttgggccgtcgccatgatcaacagacaagagatcggcggaccccggtcctacacaattgccgtggcttctctcggcaaaggcgtggcctgtaatcccgcctgctttatcacacagctgctgcccgtgaagagaaagctgggcttttacgagtggaccagcagactgcggagccacatcaatcctaccggcacagtgctgctgcagctggaaaacacaatgcagatgagcctgaaggacctgctgtgatgatgtacaaagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 13 - protein, 429 aa, Artificial Sequence; identical to SEQ ID NO: 2
  Synthetic Construct

SEQ ID NO: 14 - DNA, 4294 bp, Artificial Sequence
  CB7.CI.hGLAco.WPRE.RBG
  repeat_region (1)..(130): 5′ ITR
  repeat_region (198)..(579): CMV IE promoter
  promoter (582)..(863): CB promoter
  TATA_signal (582)..(863): TATA
  Intron (956)..(1928): chicken beta-actin intron
  CDS (1950)..(3242): hGLAco
  misc_feature (3317)..(3905): WPRE
  polyA_signal (3950)..(4076): Rabbit globin poly A
  repeat_region (4165)..(4294): 3′ ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaatagcttcgaattcgccaccatgcagctgagaaatcccgagctgcacctgggctgtgccctggctctgagatttctggccctggtgtcttgggacatccctggcgctagagccctggataacggcctggccagaacacctacaatgggctggctgcactgggagagattcatgtgcaacctggactgccaagaggaacccgacagctgcatcagcgagaagctgttcatggaaatggccgagctgatggtgtccgaaggctggaaggacgccggctacgagtacctgtgcatcgacgactgttggatggcccctcagagagactctgagggcagactgcaggccgatcctcagagatttccccacggcattagacagctggccaactacgtgcacagcaagggcctgaagctgggcatctacgccgacgtgggcaacaagacctgtgccggctttcctggcagcttcggctactacgatatcgacgcccagaccttcgccgattggggagtcgatctgctgaagttcgacggctgctactgcgacagcctggaaaatctggccgacggctacaagcacatgtcactggccctgaatcggaccggccgcagcatcgtgtactcttgcgagtggcccctgtatatgtggcccttccagaagcctaactacaccgagatcagacagtactgcaaccactggcggaacttcgccgacatcgacgatagctggaagtccatcaagagcatcctggactggaccagcttcaatcaagagcggatcgtggacgtggcaggacctggcggatggaacgatcctgacatgctggtcatcggcaacttcggcctgagctggaaccagcaagtgacccagatggccctgtgggccattatggccgctcctctgttcatgagcaacgacctgagacacatcagccctcaggccaaggctctgctgcaggacaaggatgtgatcgctatcaaccaggatcctctgggcaagcagggctaccagctgagacagggcgacaatttcgaagtgtgggaaagacccctgagcggactggcttgggccgtcgccatgatcaacagacaagagatcggcggaccccggtcctacacaattgccgtggcttctctcggcaaaggcgtggcctgtaatcccgcctgctttatcacacagctgctgcccgtgaagagaaagctgggcttttacgagtggaccagcagactgcggagccacatcaatcctaccggcacagtgctgctgcagctggaaaacacaatgcagatgagcctgaaggacctgctgtgatgatgtacaagtaaagatctgcggccgcgtggtacctctagagtcgacccgggcggcctcgaatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 15 - protein, 429 aa, Artificial Sequence; identical to SEQ ID NO: 2
  Synthetic Construct

SEQ ID NO: 16 - DNA, 3378 bp, Artificial Sequence
  TBG.PI.hGLAco(M51C_G360C).WPRE.bGH
  repeat_region (1)..(168): 5′ ITR
  enhancer (211)..(310): alpha mic/bik
  enhancer (317)..(416): alpha mic/bik
  misc_feature (431)..(907): TBG promoter
  Intron (939)..(1071): SV40 misc intron
  CDS (1094)..(2386): hGLAco.M51C.G360C
  misc_feature (2399)..(2940): WPRE
  polyA_signal (2947)..(3161): BGH pA
  repeat_region (3211)..(3378): 3′ ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagactgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgcgccaccatgcaactgagaaatcctgaactgcacctgggctgcgccctggctctgagatttctggctctggtgtcctgggacatccctggcgctagagccctggataacggcctggccagaacacctacaatgggctggctgcactgggagagattctgctgcaacctggactgccaagaggaacccgacagctgcatcagcgagaagctgttcatggaaatggccgagctgatggtgtccgaaggctggaaggacgccggctacgagtacctgtgcatcgacgactgttggatggcccctcagagagactctgagggcagactgcaggccgatcctcagagatttccccacggcattagacagctggccaactacgtgcacagcaagggcctgaagctgggcatctacgccgacgtgggcaacaagacctgtgccggctttcctggcagcttcggctactacgatatcgacgcccagaccttcgccgattggggagtcgatctgctgaagttcgacggctgctactgcgacagcctggaaaatctggccgacggctacaagcacatgtctctggccctgaatcggaccggcagatccatcgtgtacagctgcgagtggcccctgtacatgtggcccttccagaagcctaactacaccgagatcagacagtactgcaaccactggcggaacttcgccgacatcgacgatagctggaagtccatcaagagcatcctggactggaccagcttcaatcaagagcggatcgtggacgtggcaggacctggcggatggaacgatcctgacatgctggtcatcggcaacttcggcctgagctggaaccagcaagtgacccagatggccctgtgggccattatggccgctcctctgttcatgagcaacgacctgagacacatcagccctcaggccaaggctctgctgcaggacaaggatgtgatcgctatcaaccaggatcctctgggcaagcagggctaccagctgagacagggcgacaatttcgaagtgtgggaaagacccctgagcggactggcttgggccgtcgccatgatcaaccggcaagagatttgcggccccagatcctacacaatcgccgtggccagtctcggcaaaggcgtggcatgtaatcccgcctgcttcatcacacagctgctgcccgtgaagagaaagctgggcttttacgagtggaccagcagactgcggagccacatcaatcctaccggcacagtgctgctgcagctggaaaacaccatgcagatgagcctgaaggacctgctgtgatagaagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 17 - protein, 429 aa, Artificial Sequence
  Synthetic Construct
MQLRNPELHLGCALALRFLALVSWDIPGARALDNGLARTPTMGWLHWERFCCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEICGPRSYTIAVASLGKGVACNPACFITQLLPVKRKLGFYEWTSRLRSHINPTGTVLLQLENTMQMSLKDLL

SEQ ID NO: 18 - DNA, 4240 bp, Artificial Sequence
  CB7.CI.hGLAco(M51C_G360C).WPRE.RBG
  repeat_region (1)..(130): 5′ ITR
  misc_feature (198)..(579): CMV IE promoter
  promoter (582)..(863): CB promoter
  TATA_signal (836)..(839): TATA
  Intron (956)..(1928): chicken beta-actin intron
  CDS (1958)..(3250): hGLAco.M51C.G360C
  misc_feature (2363)..(2382): P18
  misc_feature (3263)..(3851): WPRE
  misc_feature (3619)..(3638): P19
  polyA_signal (3896)..(4022): Rabbit globin poly A
  repeat_region (4111)..(4240): 3′ ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaatagcttcgaattcgcggccgcgccaccatgcaactgagaaatcctgaactgcacctgggctgcgccctggctctgagatttctggctctggtgtcctgggacatccctggcgctagagccctggataacggcctggccagaacacctacaatgggctggctgcactgggagagattctgctgcaacctggactgccaagaggaacccgacagctgcatcagcgagaagctgttcatggaaatggccgagctgatggtgtccgaaggctggaaggacgccggctacgagtacctgtgcatcgacgactgttggatggcccctcagagagactctgagggcagactgcaggccgatcctcagagatttccccacggcattagacagctggccaactacgtgcacagcaagggcctgaagctgggcatctacgccgacgtgggcaacaagacctgtgccggctttcctggcagcttcggctactacgatatcgacgcccagaccttcgccgattggggagtcgatctgctgaagttcgacggctgctactgcgacagcctggaaaatctggccgacggctacaagcacatgtctctggccctgaatcggaccggcagatccatcgtgtacagctgcgagtggcccctgtacatgtggcccttccagaagcctaactacaccgagatcagacagtactgcaaccactggcggaacttcgccgacatcgacgatagctggaagtccatcaagagcatcctggactggaccagcttcaatcaagagcggatcgtggacgtggcaggacctggcggatggaacgatcctgacatgctggtcatcggcaacttcggcctgagctggaaccagcaagtgacccagatggccctgtgggccattatggccgctcctctgttcatgagcaacgacctgagacacatcagccctcaggccaaggctctgctgcaggacaaggatgtgatcgctatcaaccaggatcctctgggcaagcagggctaccagctgagacagggcgacaatttcgaagtgtgggaaagacccctgagcggactggcttgggccgtcgccatgatcaaccggcaagagatttgcggccccagatcctacacaatcgccgtggccagtctcggcaaaggcgtggcatgtaatcccgcctgcttcatcacacagctgctgcccgtgaagagaaagctgggcttttacgagtggaccagcagactgcggagccacatcaatcctaccggcacagtgctgctgcagctggaaaacaccatgcagatgagcctgaaggacctgctgtgatagaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 19 - protein, 429 aa, Artificial Sequence; identical to SEQ ID NO: 17
  Synthetic Construct

SEQ ID NO: 20 - DNA, 2211 bp, adeno-associated virus human 68
  CDS (1)..(2211)
atggctgccgatggttatcttccagattggctcgaggacaacctcagtgaaggcattcgcgagtggtgggctttgaaacctggagcccctcaacccaaggcaaatcaacaacatcaagacaacgctcggggtcttgtgcttccgggttacaaataccttggacccggcaacggactcgacaagggggagccggtcaacgaagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgtgggtattggcaaatcgggtgcacagcccgctaaaaagagactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcaaagactcatcaacaacaactggggattccggcctaagcgactcaacttcaagctcttcaacattcaggtcaaagaggttacggacaacaatggagtcaagaccatcgctaataaccttaccagcacggtccaggtcttcacggactcagactatcagctcccgtacgtgctcgggtcggctcacgagggctgcctcccgccgttcccagcggacgttttcatgattcctcagtacgggtatctaacgcttaatgatggaagccaagccgtgggtcgttcgtccttttactgcctggaatatttcccgtcgcaaatgctaagaacgggtaacaacttccagttcagctacgagtttgagaacgtacctttccatagcagctatgctcacagccaaagcctggaccgactcatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactaccaacccagtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggctttcaacaaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtttattctgaaccccgccccattggcaccagatacctgactcgtaatctgtaa

SEQ ID NO: 21 - protein, 736 aa, adeno-associated virus human 68
MAADGYLPDWLEDNLSEGIREWWALKPGAPQPKANQQHQDNARGLVLPGYKYLGPGNGLDKGEPVNEADAAALEHDKAYDQQLKAGDNPYLKYNHADAEFQERLKEDTSFGGNLGRAVFQAKKRLLEPLGLVEEAAKTAPGKKRPVEQSPQEPDSSVGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 22 - DNA, 2208 bp, adeno-associated virus 9
  CDS (1)..(2208): AAV9 VP1 Capsid
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctg

SEQ ID NO: 23 - protein, 736 aa, adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 24 - DNA, 130 bp, adeno-associated virus 2
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 25 - DNA, 130 bp, adeno-associated virus 2
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 26 - DNA, 973 bp, Artificial Sequence
  synthetic construct
gtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaag

SEQ ID NO: 27 - DNA, 589 bp, Artificial Sequence
  synthetic construct
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 28 - DNA, 127 bp, Artificial Sequence
  synthetic construct
gatctttttccctctgccaaaaattatggggacatcatgaagccccttgagcatctgacttctggctaataaaggaaatttattttcattgcaatagtgtgttggaattttttgtgtctctcactcg

SEQ ID NO: 29 - DNA, 282 bp, Artificial Sequence
  synthetic construct
tggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgg

SEQ ID NO: 30 - DNA, 382 bp, Artificial Sequence
  synthetic construct
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattac

SEQ ID NO: 31 - DNA, 22 bp, Artificial Sequence
  miR target sequence
agtgaattctaccagtgccata

SEQ ID NO: 32 - DNA, 24 bp, Artificial Sequence
  miR target sequence
agtgtgagttctaccattgccaaa